(19) United States
(12) Patent Application Publication
(10) Pub. No.: US 2025/0259732 A1
(43) Pub. Date: Aug. 14, 2025

(54) INFORMATION PROCESSING SYSTEM, INFORMATION PROCESSING PROGRAM, AND INFORMATION PROCESSING METHOD

(71) Applicants: SHINSHU UNIVERSITY, Matsumoto (JP); WELLNAS. CO.,LTD, Suginami (JP)

(72) Inventor: Kozo NAKAMURA, Minamiminowa (JP)

(21) Appl. No.: 18/841,904

(22) PCT Filed: Feb. 27, 2023
(86) PCT No.: PCT/JP2023/007162

§ 371 Date: Apr. 24, 2025

(30) Foreign Application Priority Data
Feb. 27, 2022 (JP) 2022-028755

(51) Int. Cl. G16H 20/60

(52) U.S. Cl. CPC G16H 20/60

(57) ABSTRACT

To manage health in consideration of the individual difference. An information processing system according to the present invention includes: a measured value storage unit configured to store a measured value linked to a goal to be achieved by a user; an intake storage unit configured to store nutrients intakes of the user; an analysis unit configured to analyze a relationship between the nutrients intake and the measured value to specify nutrients that contribute to improving the measured value (improvement factor) and nutrients that contribute to deteriorating the measured value (deterioration factor); a meal menu storage unit configured to store information specifying the nutrient content in a meal menu; and a meal menu recommendation unit configured to modify the meal menu by at least one of increasing the improvement factor content and decreasing of the deterioration factor content, and recommend the modified meal menu.

### TECHNICAL FIELD

The present invention relates to an information processing system, an information processing program, and an information processing method.

### BACKGROUND ART

Advice is offered to improve dietary lifestyle. For example, Patent Literature 1 discloses a system that analyzes dietary information or vital data to offer advice.

### CITATION LIST

### Patent Literature

Patent Literature 1: JP 2004-302498 A

### SUMMARY OF INVENTION

### Technical Problem

However, the system disclosed in Patent Literature 1 necessitates the preparation of advice in the form of a database in advance, so making it difficult to consider the individual difference.

The present invention is made in view of such a situation and is intended to provide a technology capable of managing health considering the individual difference.

### Solution to Problem

A primary invention of the present invention to solve the above problem including: a measured value storage unit configured to store a measured value linked to a goal to be achieved by a user; an intake storage unit configured to store intakes of a plurality of nutrients ingested by the user; an analysis unit configured to analyze a relationship between the intake of the nutrient and the measured value to specify an improvement factor that is the nutrient contributing to improvement of the measured value and a deterioration factor that is the nutrient contributing to deterioration of the measured value; a meal menu storage unit configured to store information specifying a content of the nutrient included in a meal menu; and a meal menu recommendation unit configured to modify the meal menu by at least one of increasing the content of the improvement factor and decreasing the content of the deterioration factor, and recommend the modified meal menu.

Other problems and solutions thereto disclosed herein will become apparent from preferred embodiments and drawings of the invention.

### Advantageous Effects of Invention

According to the present invention, it is possible to manage health in consideration of the individual difference.

### BRIEF DESCRIPTION OF DRAWINGS

FIG. 1 is a diagram illustrating an overall configuration example of an information processing system according to a first embodiment.

FIG. 2 is a diagram illustrating a hardware configuration example of a server device 1 according to the first embodiment.

FIG. 3 is a diagram illustrating a functional configuration example of the server device 1 according to the first embodiment.

FIG. 4 is a diagram illustrating a configuration example of information stored in a meal menu storage unit 135 according to the first embodiment.

FIG. 5 is a diagram illustrating a configuration example of information stored in an ingredient information storage unit 136 according to the first embodiment.

FIG. 6 is a diagram illustrating an example of an analysis result of an analysis unit 115 according to the first embodiment.

FIG. 7 is a diagram illustrating an example of a formula of a prediction model generated by the analysis unit 115 according to the first embodiment.

FIG. 8 is a diagram illustrating an example of processing in the server device 1 according to the first embodiment.

FIG. 9 is a diagram illustrating a test schedule (feasibility test) in 2020 according to a first example.

FIG. 10 is a diagram illustrating a test schedule (feasibility test) in 2021 according to the first example.

FIG. 11 is a diagram illustrating a test subject characteristic according to the first example.

FIG. 12 is a diagram illustrating a test schedule (clinical test) in 2020 according to the first example.

FIG. 13 is a diagram illustrating a test schedule (clinical test) in 2021 according to the first example.

FIG. 14 is a graph illustrating comparison of the amounts of energy and nutrients ingested by a test subject from a test food during an intervention period.

FIG. 15 is a graph illustrating comparison of the amounts of energy and nutrients ingested by the test subject from the test food during the intervention period.

FIG. 16 is a graph illustrating comparison of the amounts of energy and nutrients ingested by the test subject from the test food during the intervention period.

FIG. 17 is a graph illustrating comparison of the amounts of energy and nutrients ingested by the test subject from the test food during the intervention period.

FIG. 18 is a graph illustrating comparison of the amounts of energy and nutrients ingested by the test subject from the test food during the intervention period.

FIG. 19 is a graph illustrating comparison of the amounts of energy and nutrients ingested by the test subject from the test food during the intervention period.

FIG. 20 is a graph illustrating comparison between average values of wake-up weights between an observation period and the intervention period.

FIG. 21 is a graph illustrating comparison between average values of wake-up weights between the observation period and the intervention period.

FIG. 22 is a graph illustrating comparison between average values of wake-up weights between the observation period and the intervention period.

FIG. 23 is a graph illustrating a body fat percentage, a skeletal muscle percentage, and a basal metabolic rate measured by a body weight and composition monitor.

FIG. 24 is a graph illustrating the body fat percentage, the skeletal muscle percentage, and the basal metabolic rate measured by the body weight and composition monitor.

FIG. 25 is a diagram illustrating a measurement result of the amount of exercise (step count) measured by an activity meter.

FIG. 26 is a diagram illustrating a measurement result of the amount of exercise (step count) measured by the activity meter.

FIG. 27 is a diagram illustrating the degree of intervention of a weight-related factor for a test target.

FIG. 28 is a diagram illustrating the degree of intervention of a weight-related factor for the test target.

FIG. 29 is a diagram illustrating the degree of intervention of a weight-related factor for the test target.

FIG. 30 is a diagram illustrating the degree of intervention of a weight-related factor for the test target.

FIG. 31 is a diagram illustrating the degree of intervention of a weight-related factor for the test target.

FIG. 32 is a diagram illustrating the degree of intervention of a weight-related factor for the test target.

FIG. 33 is a diagram illustrating a change in blood glucose level.

FIG. 34 is a diagram illustrating a change in total cholesterol level.

FIG. 35 is a diagram illustrating a change in HDL cholesterol level.

FIG. 36 is a diagram illustrating a change in LDL cholesterol level.

FIG. 37 is a diagram illustrating a result of multiple regression analysis of a wake-up weight and an intake nutrient amount a day before wake-up weight measurement.

FIG. 38 is a diagram illustrating comparison of an intake of energy between the observation period and the intervention period.

FIG. 39 is a diagram illustrating comparison of intakes between the observation period and the intervention period.

FIG. 40 is a diagram illustrating comparison of intakes between the observation period and the intervention period.

FIG. 41 is a diagram illustrating comparison of intakes between the observation period and the intervention period.

FIG. 42 is a diagram illustrating comparison of intakes between the observation period and the intervention period.

FIG. 43 is a diagram illustrating comparison of intakes between the observation period and the intervention period.

FIG. 44 is a diagram illustrating comparison of intakes between the observation period and the intervention period.

FIG. 45 is a diagram illustrating a result of comparing measured values of the observation period and the intervention period for the wake-up weight.

FIG. 46 is a diagram illustrating a result of comparing measured values of the observation period and the intervention period for the wake-up weight.

FIG. 47 is a diagram illustrating a result of comparing measured values of the observation period and the intervention period for the wake-up weight.

FIG. 48 is a diagram illustrating a result of comparing measured values of the observation period and the intervention period for the wake-up weight.

FIG. 49 is a diagram illustrating a result of comparing measured values of the observation period and the intervention period for the wake-up weight.

FIG. 50 is a diagram illustrating a result of comparing measured values of the observation period and the intervention period for the wake-up weight.

FIG. 51 is a diagram illustrating a body composition.

FIG. 52 is a diagram illustrating a body composition.

FIG. 53 is a diagram illustrating a body composition.

FIG. 54 is a diagram illustrating a body composition.

FIG. 55 is a diagram illustrating a body composition.

FIG. 56 is a diagram illustrating a body composition.

FIG. 57 is a diagram illustrating the degrees of intervention of related factors.

FIG. 58 is a diagram illustrating the degrees of intervention of related factors.

FIG. 59 is a diagram illustrating the degrees of intervention of related factors.

FIG. 60 is a diagram illustrating the degrees of intervention of related factors.

FIG. 61 is a diagram illustrating a blood cholesterol level.

FIG. 62 is a diagram illustrating a test schedule in 2020 according to a second example.

FIG. 63 is a diagram illustrating a result of multiple regression analysis.

FIG. 64 is a diagram illustrating a condition in each analysis.

FIG. 65 is a diagram illustrating an example of an optimal personal diet.

FIG. 66 is a diagram illustrating the degrees of intervention of memory-related factors for the test target.

FIG. 67 is a diagram illustrating the degree of intervention of a memory-related factor for the test target.

FIG. 68 is a diagram illustrating the degree of intervention of a memory-related factor for the test target.

FIG. 69 is a diagram illustrating the degree of intervention of a memory-related factor for the test target.

FIG. 70 is a diagram illustrating the degrees of intervention of memory-related factors for the test target.

FIG. 71 is a diagram illustrating the degree of intervention of a memory-related factor for the test target.

FIG. 72 is a diagram illustrating the degree of intervention of a memory-related factor for the test target.

FIG. 73 is a diagram illustrating comparison of intakes between the observation period and the intervention period.

FIG. 74 is a diagram illustrating comparison of intakes between the observation period and the intervention period.

FIG. 75 is a diagram illustrating comparison of intakes between the observation period and the intervention period.

FIG. 76 is a diagram illustrating comparison of intakes between the observation period and the intervention period.

FIG. 77 is a diagram illustrating comparison of intakes between the observation period and the intervention period.

FIG. 78 is a diagram illustrating comparison of intakes between the observation period and the intervention period.

FIG. 79 is a diagram illustrating comparison of intakes between the observation period and the intervention period.

FIG. 80 is a diagram illustrating a change in memory test score.

FIG. 81 is a diagram illustrating comparison of an average of memory test scores between the observation period and the intervention period.

FIG. 82 is a diagram illustrating comparison of a distribution of the memory test scores between the observation period and the intervention period.

FIG. 83 is a diagram illustrating a result of multiple regression analysis.

FIG. 84 is a diagram illustrating the degree of intervention of a memory-related factor for the test target.

FIG. 85 is a diagram illustrating comparison of intakes between the observation period and the intervention period.

FIG. 86 is a diagram illustrating a change in memory test score.

FIG. 87 is a diagram illustrating comparison of an average of memory test scores between the observation period and the intervention period.

FIG. 88 is a diagram illustrating comparison of a distribution of the memory test scores between the observation period and the intervention period.

FIG. 89 is a diagram illustrating a test schedule according to a third example.

FIG. 90 is a diagram illustrating a result of multiple regression analysis.

FIG. 91 is a diagram illustrating an example of an optimal personal diet.

FIG. 92 is a diagram illustrating the degrees of intervention of endurance-related factors for the test target.

FIG. 93 is a diagram illustrating the degrees of intervention of endurance-related factors for the test target.

FIG. 94 is a diagram illustrating the degrees of intervention of endurance-related factors for the test target.

FIG. 95 is a diagram illustrating the degrees of intervention of endurance-related factors for the test target.

FIG. 96 is a diagram illustrating comparison of intakes between the observation period and the intervention period.

FIG. 97 is a diagram illustrating comparison of intakes between the observation period and the intervention period.

FIG. 98 is a diagram illustrating comparison of intakes between the observation period and the intervention period.

FIG. 99 is a diagram illustrating comparison of intakes between the observation period and the intervention period.

FIG. 100 is a diagram illustrating a change in resting heart rate.

FIG. 101 is a diagram illustrating comparison of an average of resting heart rates between the observation period and the intervention period.

FIG. 102 is a diagram illustrating comparison of a blood oxygen level at the end of a 45-minute jog between the observation period and the intervention period.

FIG. 103 is a diagram illustrating comparison of an average of blood oxygen levels at the end of a 45-minute jog between the observation period and the intervention period.

FIG. 104 is a diagram illustrating the number of shuttle runs during the observation period and the intervention period.

FIG. 105 is a diagram illustrating comparison of an average of the numbers of 20-m shuttle runs between the observation period and the intervention period.

FIG. 106 is a diagram illustrating a change in resting heart rate during a post-observation period.

FIG. 107 is a diagram illustrating a functional configuration example of a server device 1 according to a second embodiment.

FIG. 108 is a diagram illustrating a configuration example of user information.

### DESCRIPTION OF EMBODIMENTS

Illustrative embodiments of the present invention are in detail described. A health management system according to an embodiment of the present invention has a configuration as below.

### [Item 1]

An information processing system including:
a measured value storage unit configured to store a measured value linked to a goal to be achieved by a user;
an intake storage unit configured to store intakes of a plurality of nutrients ingested by the user;
an analysis unit configured to analyze a relationship between the intake of the nutrient and the measured value to specify an improvement factor that is the nutrient contributing to improvement of the measured value and a deterioration factor that is the nutrient contributing to deterioration of the measured value;
a meal menu storage unit configured to store information specifying a content of the nutrient included in a meal menu; and
a meal menu recommendation unit configured to modify the meal menu by at least one of increasing the content of the improvement factor and decreasing the content of the deterioration factor, and recommend the modified meal menu.

### [Item 2]

The information processing system according to Item 1, further including:
an ingredient information storage unit configured to store the content of the nutrient for each ingredient,
in which the meal menu storage unit stores the ingredient included in the meal menu, and
the meal menu recommendation unit recommends the modified meal menu by performing at least one of:
selecting, from the ingredient information storage unit, a second ingredient including a larger amount of the nutrient that is the improvement factor than at least one first ingredient included in the meal menu, and modifying the meal menu so as to change the first ingredient to the selected second ingredient;
selecting, from the ingredient information storage unit, a third ingredient including a smaller amount of the nutrient that is the deterioration factor than the first ingredient, and modifying the meal menu so as to change the first ingredient to the selected third ingredient;
selecting, from the ingredient information storage unit, a fourth ingredient including a larger amount of the nutrient that is the improvement factor than the first ingredient and including a smaller amount of the nutrient that is the deterioration factor than the first ingredient, and modifying the meal menu so as to change the first ingredient to the selected fourth ingredient;
selecting, from the ingredient information storage unit, a fifth ingredient including the nutrient that is the improvement factor, and modifying the meal menu so as to add the selected fifth ingredient; and
selecting, from the ingredient information storage unit, a sixth ingredient including the nutrient that is the deterioration factor, and modifying the meal menu so as to delete the selected sixth ingredient.

### [Item 3]

The information processing system according to Item 1 or 2, in which
the analysis unit calculates the degree of improvement contributing to improvement for the improvement factor, and calculates the degree of deterioration contributing to deterioration for the deterioration factor, and
the meal menu recommendation unit
calculates a first value obtained by subtracting a value obtained by multiplying the degree of deterioration relating to the deterioration factor included in the meal menu by the content from a value obtained by multiplying the degree of improvement relating to the improvement factor included in the meal menu by the content, and
modifies the meal menu such that a second value obtained by subtracting a value obtained by multiplying the degree of deterioration relating to the deterioration factor included in the modified meal menu by the content from a value obtained by multiplying the degree of improvement relating to the improvement factor included in the modified meal menu by the content is larger than the first value.

### [Item 4]

The information processing system according to Item 1 or 2, in which
the analysis unit calculates the degree of improvement contributing to improvement for the improvement factor, and calculates the degree of deterioration contributing to deterioration for the deterioration factor, and
the meal menu recommendation unit
calculates a first improvement value obtained by subtracting a value obtained by multiplying the intake relating to the deterioration factor stored in the intake storage unit by the degree of deterioration from a value obtained by multiplying the intake relating to the improvement factor stored in the intake storage unit by the degree of improvement, and
modifies the meal menu such that a second improvement value obtained by subtracting a value obtained by multiplying the degree of deterioration relating to the deterioration factor included in the modified meal menu by the content from a value obtained by multiplying the degree of improvement relating to the improvement factor included in the modified meal menu by the content is larger than the first improvement value.

### [Item 5]

The information processing system according to any one of Items 1 to 4, further including:
an activity amount storage unit configured to store an activity amount of an activity performed by the user; and
a target value input unit configured to receive an input of a target value of the measured value,
in which the analysis unit creates a prediction model obtained by analyzing a relationship between the content of the nutrient, the activity amount, and the measured value, and
the meal menu recommendation unit determines the activity amount according to a difference between a predicted value obtained by applying a content of the nutrient included in the modified meal menu to the prediction model and the target value, and recommends the determined activity amount together with the meal menu.

### [Item 6]

The information processing system according to any one of Items 1 to 5, in which
the analysis unit periodically performs analysis to specify the improvement factor and the deterioration factor, and the meal menu recommendation unit recommends the modified meal menu.

### [Item 7]

An information processing system including:
a measured value storage unit configured to store a measured value for a first user linked to a goal to be achieved by the first user;
an intake storage unit configured to store intakes of a plurality of nutrients ingested by the first user;
an analysis unit configured to analyze a relationship between the intake of the nutrient and the measured value for the first user to specify an improvement factor that is the nutrient contributing to improvement of the measured value and a deterioration factor that is the nutrient contributing to deterioration of the measured value;
a meal menu storage unit configured to store information specifying a content of the nutrient included in a meal menu;
a similar user specifying unit configured to specify a similar user who is the first user similar to an attribute of a second user; and
a meal menu recommendation unit configured to modify the meal menu by at least one of increasing the content of the improvement factor specified for the similar user and decreasing the content of the deterioration factor specified for the similar user, and recommend the modified meal menu to the second user.

### [Item 8]

An information processing system including:
a measured value storage unit configured to store a measured value for a first user linked to a goal to be achieved by the first user;
an intake storage unit configured to store intakes of a plurality of nutrients ingested by the first user;
a meal menu storage unit configured to store information specifying a content of the nutrient included in a meal menu;
a similar user specifying unit configured to specify a similar user who is the first user similar to an attribute of a second user;
an analysis unit configured to read the measured value and the intake corresponding to the similar user from the measured value storage unit and the intake storage unit and analyze a relationship between the intake of the nutrient and the measured value to specify an improvement factor that is the nutrient contributing to improvement of the measured value and a deterioration factor that is the nutrient contributing to deterioration of the measured value; and
a meal menu recommendation unit configured to modify the meal menu by at least one of increasing the content of the improvement factor and decreasing the content of the deterioration factor, and recommend the modified meal menu to the second user.

### [Item 9]

An information processing method executed by a computer, the information processing method including:
storing a measured value linked to a goal to be achieved by a user;
storing intakes of a plurality of nutrients ingested by the user;
analyzing a relationship between the intake of the nutrient and the measured value to specify an improvement factor that is the nutrient contributing to improvement of the measured value and a deterioration factor that is the nutrient contributing to deterioration of the measured value;
storing information specifying a content of the nutrient included in a meal menu; and
modifying the meal menu by at least one of increasing the content of the improvement factor and decreasing the content of the deterioration factor, and recommending the modified meal menu.

### [Item 10]

A program for causing a computer to execute:
storing a measured value linked to a goal to be achieved by a user;
storing intakes of a plurality of nutrients ingested by the user;
analyzing a relationship between the intake of the nutrient and the measured value to specify an improvement factor that is the nutrient contributing to improvement of the measured value and a deterioration factor that is the nutrient contributing to deterioration of the measured value;
storing information specifying a content of the nutrient included in a meal menu; and
modifying the meal menu by at least one of increasing the content of the improvement factor and decreasing the content of the deterioration factor, and recommending the modified meal menu.

Hereinafter, preferred embodiments of the present disclosure will be described in detail with reference to the accompanying drawings. In the present specification and the drawings, components having substantially the same functional configuration are denoted by the same reference numerals, and an overlapping description is omitted.

### First Embodiment

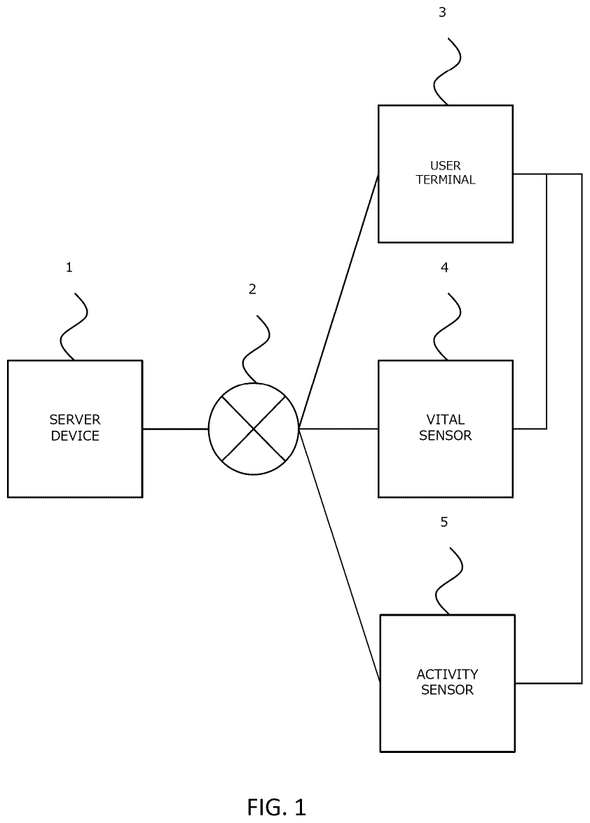

FIG. 1 is a diagram illustrating an overall configuration of a health support system. As illustrated in FIG. 1, the health support system includes a server device 1, a user terminal 3, a vital sensor 4, and an activity sensor 5. The server device 1 is connected to the user terminal 3, the vital sensor 4, and the activity sensor 5 via a network 2. Although only one server device 1, one user terminal 3, one vital sensor 4, and one activity sensor 5 are illustrated, it goes without saying that there may be more server devices 1, more user terminals 3, more vital sensors 4, and more activity sensors 5. The network 3 is, for example, the Internet, and is constructed with the Ethernet (registered trademark), a public telephone network, a dedicated telephone network, a mobile telephone network, a wireless communication path, or the like.

### ==Server Device 1==

The server device 1 is a computer that provides, to a user, information regarding an element necessary for reaching a goal to be achieved in consideration of an individual difference. The server device 1 is, for example, a personal computer or a workstation. The server device 1 can also be implemented as a virtual computer by cloud computing.

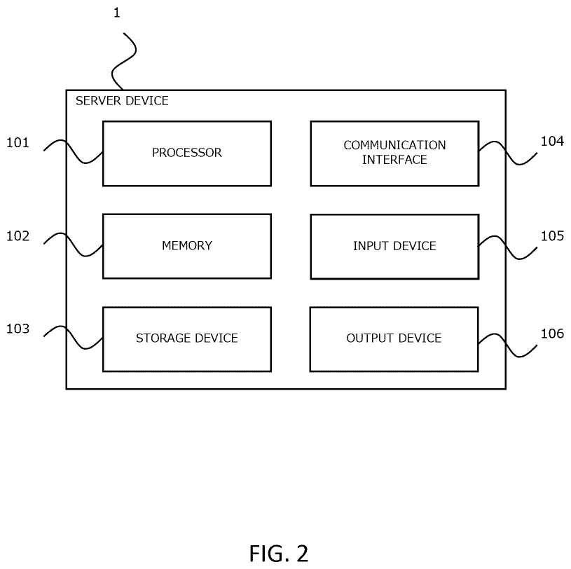

FIG. 2 is a diagram illustrating a hardware configuration example of the server device 1 according to the present embodiment. The server device 1 includes a processor 101, a memory 102, a storage device 103, a communication interface 104, an input device 105, and an output device 106. The storage device 103 is, for example, a hard disk drive, a solid state drive, a flash memory, or the like that stores various data and programs. The communication interface 104 is an interface for connection to the network 2, and is, for example, an adapter for connection to the Ethernet (registered trademark), a modem for connection to a public telephone network, a wireless communication device for wireless communication, a universal serial bus (USB) connector for serial communication, an RS232C connector, or the like. The input device 105 is a device that receives data input through, for example, a keyboard, a mouse, a touch panel, a button, a microphone, or the like. The output device 106 includes, for example, a display, a printer, a speaker, and the like that output data.

### ==User Terminal 3==

The user terminal 3 is a computer operated by the user who is a health support target. The user terminal 3 is, for example, a smartphone, a tablet computer, or a personal computer, but is not limited thereto. The user can access the server device 1 by, for example, an application or a web browser executed on the user terminal 3. The user terminal 3 is communicably connected to the vital sensor 4 and the activity sensor 5. The user terminal 3, the vital sensor 4, and the activity sensor 5 communicate with each other by, for example, Bluetooth (registered trademark) Low Energy (BLE) or serial communication.

### ==Vital Sensor 4=

The vital sensor 4 is a device that acquires vital information of the user. The vital sensor 4 includes, for example, a blood pressure monitor, a pulse oximeter, and the like.

### ==Activity Sensor 5=

The activity sensor 5 is a device that acquires activity information regarding an activity of the user. The activity sensor 4 includes, for example, a pedometer, an activity meter, and the like.

The vital sensor 4 and the activity sensor 5 may be the same device, or the vital sensor 4 and the activity sensor 5 may be partially or entirely mounted as a part of the user terminal 3.

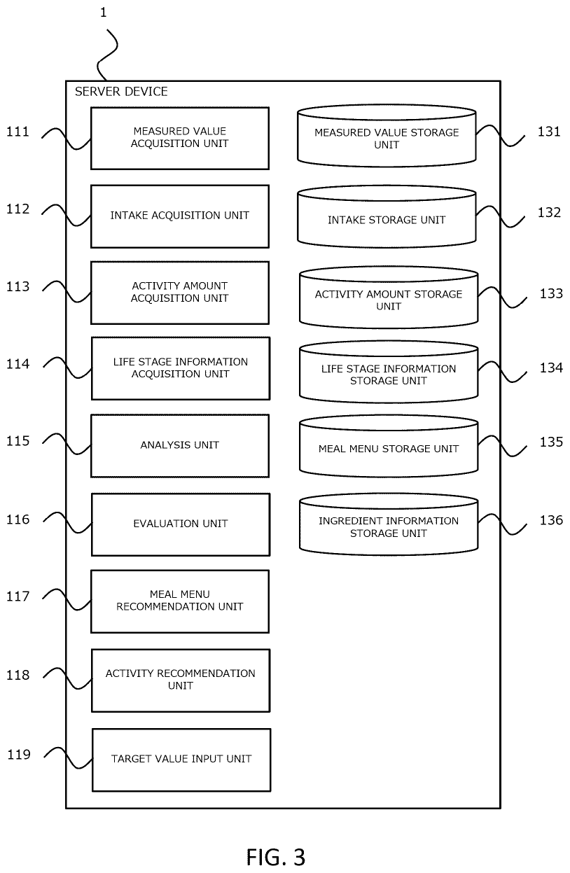

FIG. 3 is a block diagram illustrating a functional configuration of the server device 1. As illustrated in FIG. 3, the server device 1 includes processing units of a measured value acquisition unit 111, an intake acquisition unit 112, an activity amount acquisition unit 113, a life stage information acquisition unit 114, an analysis unit 115, an evaluation unit 116, a meal menu recommendation unit 117, and an activity recommendation unit 118, and storage units of a measured value storage unit 131, an intake storage unit 132, an activity amount storage unit 133, a life stage information storage unit 134, a meal menu storage unit 135, and an ingredient information storage unit 136.

Each of the processing units is implemented by the processor 101 included in the server device 1 reading a program stored in the storage device 103 into the memory 102 and executing the program, and each of the storage units is implemented as a part of a storage area provided by the memory 102 and the storage device 103 included in the server device 1.

Here, in the present embodiment, a data configuration of the measured value storage unit 131, the intake storage unit 132, the activity amount storage unit 133, the life stage information storage unit 134, the meal menu storage unit 135, and the ingredient information storage unit 136 will be described.

The measured value storage unit 131 stores a measured value linked to the goal to be achieved by the user. The goal to be achieved includes, but is not limited to, acquisition, maintenance, and improvement of health, aesthetics, body shape, motor function, brain function, skills, endurance, and the like. It is sufficient if the measured value is any value linked to a result of an activity and is measurable. For example, a resting heart rate, an average heart rate during a 30-minute run, or a time for 1500 m may be adopted as the measured value relating to the endurance. It is sufficient if the improvement of the measured values (such improvement can be expressed by the magnitude of a value when an increase in value indicates greater improvement, the degree of deviation from a target value when approaching a predetermined value indicates greater improvement, or the smallness of the value when a decrease in value indicates greater improvement) indicates improvement of the result of the activity. The measured value for the goal to be achieved may be associated with a measurement time to store a history. Furthermore, the measured value storage unit 131 can also store a standard value (hereinafter, referred to as a measurement standard value) of the measured value for each user. The measurement standard value may be the latest measured value for the user, or may be a statistical value (an average value, a median value, or the like) of the past predetermined period. The measured value storage unit 131 may store a plurality of measured values. For example, in a case where the measured value linked to the goal to be achieved by the user is a body weight, a blood pressure, a heart rate, a time for 1500 m, and the like can be recorded. As a result, even when the user changes the goal to another goal to be achieved, it is possible to evaluate a meal menu by using data registered in the past as long as the linked measured value is registered.

The intake storage unit 132 stores an intake of a nutrient ingested by the user. The nutrient includes nutritional component labeling items based on the Food Labeling Law (calorie, proteins, fats, carbohydrates, sodium, saturated fatty acids, n-3 fatty acids, n-6 fatty acids, cholesterols, carbohydrates, sugars (monosaccharides or disaccharides, limited to non-sugar alcohols), dietary fiber, zinc, potassium, calcium, chromium, selenium, iron, copper, magnesium, manganese, molybdenum, iodine, phosphorus, niacin, pantothenic acid, biotin, vitamins A, B1, B2, B6, B12, C, D, E, and K, and folic acid), and may include an amino acid, non-nutrient component others than nutritional components, such as polyphenols and GABA, and a component specified among the nutrient components as a group composed of a plurality of components (for example, cyclodextrin as dietary fiber). The intake storage unit 132 may also store the intake of the nutrient ingested by the user along with a date and time of intake and a category such as breakfast, lunch, dinner, or snacks.

The activity amount storage unit 133 stores an activity amount of the user. The activity amount is the activity amount of the user, and includes, but is not limited to, the type and amount (the number of times and a duration) of exercise performed by the user, the type and amount (the number of times and a duration) of a test (for example, a memory test) performed by the user, and the like. The activity amount storage unit 133 can store the activity amount for each unit period (for example, the unit period may be set to one day or may be set according to the number of meals like after breakfast, after lunch, after dinner, or the like). Furthermore, the activity amount storage unit 133 can also store a standard value (hereinafter, referred to as an activity standard value) of the activity amount for each user. The activity standard value may be the latest activity amount of the user, or may be a statistical value (an average value, a median value, or the like) of the past predetermined period.

The life stage information storage unit 134 stores information regarding a life stage of the user. The life stage includes the age of the user and stages defined by age, such as childhood, adolescence, adulthood, and old age, and can include, but is not limited to, information such as affiliation or employment status such as being a student, employed, preparing for employment, unemployed, or retired, and a life style such as unmarried, married, pregnancy, maternity leave, paternity leave, or parenting (infants, toddlers, children, or students).

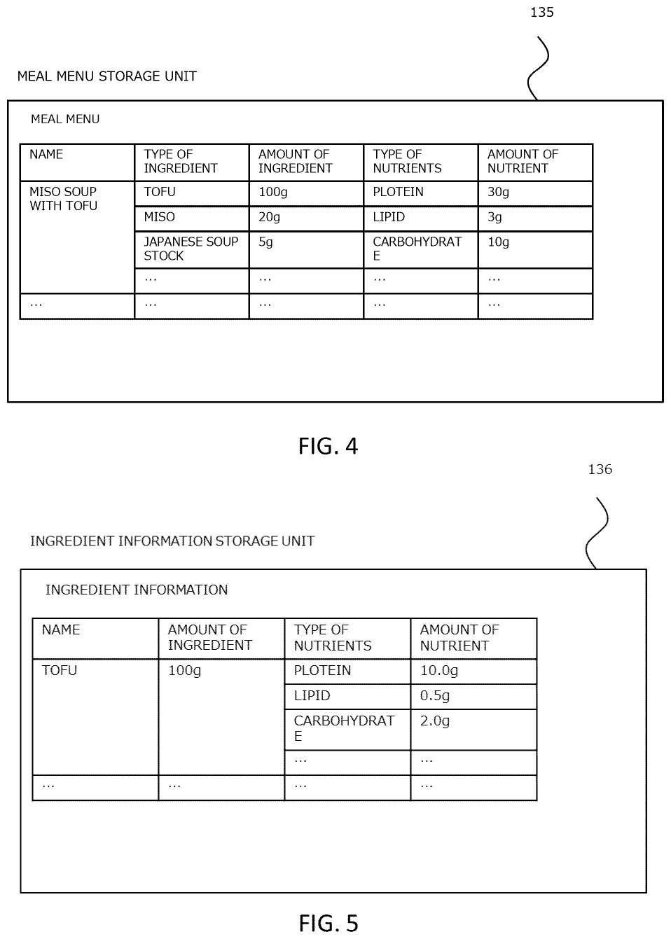

The meal menu storage unit 135 stores a meal menu. The meal menu is information including the type and content of nutrients to ingested as a meal, and in the present embodiment, for example, as illustrated in an example in FIG. 4, the meal menu can include a name, the type and amount of ingredient and seasoning included, and the type and amount of nutrients included in each ingredient and seasoning. In FIG. 4, a single dish (tofu miso soup) is illustrated as an example of the meal menu, but the meal menu may be a set including a plurality of dishes (for example, a combination of white rice, tofu miso soup, ginger pork, spinach ohitashi, and yoghurt). In addition, an ingredient that is not cooked (for example, orange or almond) or an ingredient that has not yet been cooked (for example, the rump of a cow) may be included in the “meal menu”. Further, the nutrient includes nutritional component labeling items based on the Food Labeling Law (calorie, proteins, fats, carbohydrates, sodium, saturated fatty acids, n−3 fatty acids, n−6 fatty acids, cholesterols, carbohydrates, sugars (monosaccharides or disaccharides, limited to non-sugar alcohols), dietary fiber, zinc, potassium, calcium, chromium, selenium, iron, copper, magnesium, manganese, molybdenum, iodine, phosphorus, niacin, pantothenic acid, biotin, vitamins A, B1, B2, B6, B12, C, D, E, and K, and folic acid), and may include non-nutrient component others than nutritional components, such as polyphenols and GABA, and a component specified among the nutrient components as a group composed of a plurality of components (for example, cyclodextrin as dietary fiber). In addition, a recipe of the meal menu and information regarding a cooking process (including, but not limited to, heating, cooling, boiling, or washing) that is assumed to affect the ingredients and nutrients may be stored together.

The ingredient information storage unit 136 stores ingredient information. As illustrated in an example in FIG. 5, the types and amounts of nutrients included in the ingredient are stored in the ingredient information. Seasoning information may also be stored in the ingredient information, and for each ingredient, an influence of cooking on a nutrient (decomposition by heating or the like), an influence of simultaneous cooking with other ingredients on a nutrient, and the like may also be stored.

Here, in the present embodiment, functions of the processing units of the measured value acquisition unit 111, the intake acquisition unit 112, the activity amount acquisition unit 113, the life stage information acquisition unit 114, the analysis unit 115, the evaluation unit 116, and the meal menu recommendation unit 117 will be described.

The measured value acquisition unit 111 acquires the measured value linked to the goal to be achieved by the user, and stores the measured value in the measured value storage unit 131. The measured value acquisition unit 111 can present an input form to the user terminal 3, receive an input such as selection of a value or an option from the user, and acquire the measured value. The measured value acquisition unit 111 may acquire the measured value acquired by the vital sensor 4 or the activity sensor 5 through the user terminal 3, or may acquire the measured value directly from the vital sensor 4 or the activity sensor 5 through the network 2. Furthermore, the measured value acquisition unit 111 may receive an input of the measured value from the user. The measured value acquisition unit 111 may acquire the measured value by accessing a computer (for example, a database server) that manages the measured value. The measured value acquisition unit 111 can acquire, for example, a measured value of a time measured by a stopwatch or a measured value such as a test score.

The intake acquisition unit 112 acquires information regarding an intake of a nutrient ingested by the user. The intake acquisition unit 112 presents an input form to the user terminal 3, receives an input of a meal menu that the user has eaten or selection of an option of a meal menu, and acquires information regarding a quantity eaten. The intake acquisition unit 112 may acquire information of an image (which may include a moving image) of the meal that the user has eaten from the user terminal 3, estimate a meal menu included in the image by analyzing the image, present the estimated meal menu in a correctable manner to the user terminal 3, and acquire the meal menu together with the quantity eaten. The intake acquisition unit 112 acquires, from the meal menu storage unit 135, information regarding nutrients included in the meal menu that the user has eaten, the information being acquired from the user terminal 3, and stores the information in the intake storage unit in association with a date and time of intake or a category of the meal such as breakfast, lunch, dinner, or snacks, and the meal menu.

The activity amount acquisition unit 113 acquires information regarding the amount of activity performed by the user. The activity amount acquisition unit 113 presents an input form to the user terminal 3, receives an input such as selection of a value or an option from the user, and acquires the activity amount. In addition, the activity amount acquisition unit 113 may acquire the measured value acquired by the vital sensor 4 or the activity sensor 5 through the user terminal 3, or may acquire the measured value directly from the vital sensor 4 or the activity sensor 5 through the network 2. The activity amount acquisition unit 113 may acquire information regarding the activity amount through an information terminal or the like used by a supporter of the user (examples of the supporter include, but are not limited to, a caregiver, a medical personnel, and a trainer).

The life stage information acquisition unit 114 acquires information regarding the life stage of the user. The life stage information acquisition unit 114 presents an input form to the user terminal 3, receives an input such as selection of a value or an option from the user, and acquires life stage information.

The analysis unit 115 analyzes a relationship between an intake of a nutrient and the measured value of the goal to be achieved, and specifies an improvement factor contributing to improvement of the measured value and a deterioration factor contributing to deterioration of the measured value. The Improvement factor and the deterioration factor include nutrients. Furthermore, the improvement factor and/or the deterioration factor may include the activity amount relating to the activity of the user. In the present embodiment, the analysis unit 115 analyzes a relationship between the measured value on the next day of the day on which the user takes a nutrient, and an intake of the nutrient, but a prediction time point is not limited to the next day, and may be a day after an arbitrary period such as one week, two weeks, or one month. In the following description, a data acquisition period for analysis is referred to as an “observation period”, and a period during which a meal that brings the measured value close to the target value is ingested is referred to as an “intervention period”. Not only data in the observation period but also data in the intervention period can be analyzed. The observation period and the intervention period can be any period. The intervention period can be different in length from a period for achieving the goal (hereinafter, referred to as a trial period). For example, the intervention period may be one week, and the trial period may be three months or one year. The analysis unit 115 can perform analysis by using the measured value at the end of the intervention period or after the end of the intervention period. The analysis unit 115 may perform analysis only before the start of the intervention period, or may perform analysis periodically to update a statistical model (prediction model).

The analysis unit 115 can obtain the prediction model (hereinafter, referred to as a nutrition model) by multivariate analysis with the intake of the nutrient as an explanatory variable and the measured value as an objective variable.

Furthermore, the analysis unit 115 can analyze a relationship between the intake of the nutrient and the activity amount, and the measured value to specify the improvement factor that is a nutrient and/or activity contributing to improvement of the measured value and the deterioration factor that is a nutrient and/or activity contributing to deterioration of the measured value. There may be a plurality of activities relating to the activity amount. For example, the analysis unit 115 can obtain the prediction model (hereinafter, referred to as a nutrition-activity model) by multivariate analysis using an intake of each of various nutrients such as intake energy, protein, lipid, carbohydrate, and vitamin A, and the activity amount such as a step count, an exercise duration, or the number of times of taking the memory test as explanatory variables, and using the measured value (the body weight, a cardiopulmonary function, a memory test result, or the like) as an objective variable.

The analysis unit 115 can analyze a relationship between an intake of a nutrient and the life stage, and the measured value to specify the improvement factor that is a nutrient and/or life stage contributing to improvement of the measured value, and a deterioration factor that is a nutrient and/or life stage contributing to deterioration of the measured value. For example, the analysis unit 115 can obtain the prediction model (hereinafter, referred to as a nutrient-life stage model) by multivariate analysis using an intake of each of various nutrients such as intake energy, protein, lipid, carbohydrate, and vitamin A, and a dummy variable indicating the life stage such as adulthood, student, and pregnancy as explanatory variables, and the measured value (the body weight, the cardiopulmonary function, the memory test result, or the like) as an objective variable.

The analysis unit 115 can analyze a relationship between an intake of a nutrient and a date and time when the measured value is measured (which may be converted into a time zone or a season that can be derived from the date and time), and the measured value to specify the improvement factor that is a nutrient and/or date and time contributing to improvement of the measured value, and the deterioration factor that is a nutrient and/or date and time contributing to deterioration of the measured value. For example, the analysis unit 115 can obtain the prediction model (hereinafter, referred to as a nutrition-date and time model) by multivariate analysis using the measured value (the body weight, the cardiopulmonary function, the memory test result, or the like) as an objective variable and an intake of each of various nutrients such as intake energy, protein, lipid, carbohydrate, and vitamin A, and the date and time, time zone, or season such as 9:00 AM, 17:00 PM, morning, daytime, night, spring, summer, autumn, or winter as explanatory variables.

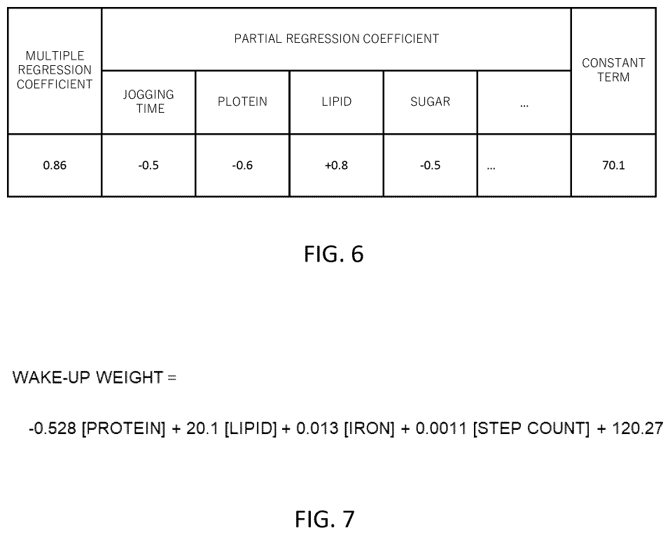

As described above, the analysis unit 115 can perform multivariate analysis with the measured value as an objective variable and at least one of the intake of the nutrient, the activity amount, the life stage, and the date and time as an explanatory variable. The analysis unit 115 may obtain a prediction model (hereinafter, referred to as a nutrient-combination model) that incorporates two or more of the activity amount, the life stage, and the date and time as an explanatory variable to be combined with the intake of the nutrient. The analysis unit 115 can also output a coefficient of each element used as the explanatory variable. FIG. 6 is a diagram illustrating an example of a result of the multivariate analysis performed by the analysis unit 115. As illustrated in FIG. 6, a regression coefficient (partial regression coefficient) may be output for the nutrient, the activity amount, the life stage, and the date and time.

The multivariate analysis according to the present embodiment includes single regression analysis, multiple regression analysis, principal component analysis, independent component analysis, factor analysis, discriminant analysis, canonical discriminant analysis, quantification theory (Groups I, II, III, and IV), multi-group quantification method type II, cluster analysis, conjoint analysis, logistic regression, multidimensional scaling (MDS), extended quantification method type I, extended quantification method type II, correspondence analysis, and covariance structure analysis.

The analysis unit 115 may store the above-described prediction model (including the nutrition model, the nutrition-activity model, the nutrition-life stage model, the nutrition-date and time model, and the nutrition-combination model) in the server device 1. In this case, the server device 1 also stores parameters such as a regression coefficient and a constant to be applied to the prediction model.

For example, the analysis unit 115 can perform multiple regression analysis to obtain the prediction model, and estimate the regression coefficient and the constant for the nutrient used as the explanatory variable and at least one of the activity amount, the life stage, and the date and time. The analysis unit 115 stores the estimated regression coefficient and the constant in the server device 1. By applying the nutrient and at least one of the activity amount, the life stage, and the date and time to the prediction model to which the estimated regression coefficient and the constant are applied, it is possible to estimate the measured value of the user when the user performs the activity by the activity amount, the user is placed at the life stage, or the measured value is measured at the date and time, in a case where the user takes a meal menu containing such a nutrient. An absolute value of the regression coefficient indicates the degree of improvement or deterioration of the measured value.

In a case where the target value of the measured value that is the goal to be achieved by the user is larger than the current measured value, the explanatory variable that is a positive or zero regression coefficient can be used as the improvement factor for improving the measured value, and the explanatory variable that is a negative regression coefficient can be used as the deterioration factor for deteriorating the measured value. In a case where the target value of the measured value that is the goal to be achieved by the user is smaller than the current measured value, a factor that is a negative or zero regression coefficient can be used as the improvement factor, and a factor that is a positive regression coefficient can be used as the deterioration factor.

The evaluation unit 116 can evaluate the meal menu on the basis of a content of the nutrient that is the improvement factor and a content of the nutrient that is the deterioration factor. For example, the evaluation unit 116 can evaluate the meal menu higher when the number of improvement factors included in the meal menu is larger, and evaluate the meal menu lower when the number of deterioration factors included in the meal menu is larger. The evaluation unit 116 can evaluate the meal menu according to, for example, a weighted average value (for example, when the improvement factor and the deterioration factor have opposite signs (positive and negative), Σ(the content of the improvement factor×the coefficient of the improvement factor)+Σ(the content of the deterioration factor×the coefficient of the deterioration factor) can be calculated, and when the coefficient indicates the “degree” of improvement or deterioration, such as the absolute value of the regression coefficient by multivariate regression, the calculation can be made as Σ(the content of the improvement factor×the coefficient of the improvement factor)−Σ(the content of the deterioration factor×the coefficient of the deterioration factor) can be calculated) of the contents of the nutrients using the coefficients of the improvement factor and the deterioration factor.

The evaluation unit 116 can also evaluate the meal menu on the basis of the prediction model generated by the analysis unit 115. For example, the evaluation unit 116 can receive an input of the target value of the measured value corresponding to the objective variable of the prediction model from the user terminal 3, and calculate an intake (hereinafter, referred to as a predicted intake) of a nutrient whose predicted value estimated by the prediction model is the target value. The evaluation unit 116 can evaluate a meal menu having a nutrient amount close to the predicted intake higher, and can evaluate a meal menu having a nutrient amount deviating from the predicted intake lower. The evaluation unit 116 can assign an evaluation value to each meal menu for each prediction model. The evaluation value is only required to be obtained by quantifying the degree of deviation from the calculated predicted intake, but is not limited thereto.

The evaluation unit 116 may evaluate a combination of meal menus on the basis of the prediction model. It is sufficient if the evaluation unit 116 evaluates a combination of meal menus whose total nutrient amount obtained by adding nutrient amounts in the plurality of meal menus is close to the predicted intake higher, and evaluates a combination of meal menus whose total nutrient amount obtained by adding the nutrient amounts in the plurality of meal menus deviates from the predicted intake lower. It is sufficient if the evaluation unit 116 assigns an evaluation value to each combination of meal menus for each prediction model.

The evaluation unit 116 may evaluate the activity amount, the life stage, or the date and time, the activity amount, and the meal menu on the basis of the prediction model generated by the analysis unit 115. It is sufficient if the evaluation method is performed according to the above-described meal menu evaluation method.

The target value input unit 119 receives an input of the target value of the measured value that is the goal to be achieved by the user. For example, an input of a target value of the body weight, a target value of a time for a short-distance run, a target value of a test score, and the like can be received.

The meal menu recommendation unit 117 can select a meal menu to be recommended to the user according to the evaluation value of the meal menu and present the meal menu in the user terminal 3. For example, the meal menu recommendation unit 117 can cause the evaluation unit 116 to calculate the evaluation value for each meal menu stored in the meal menu storage unit 135, select a predetermined number (the number may be 1 or any number such as 10) of single meal menus or combinations of a plurality of meal menus in descending order of the evaluation value, and present the selected meal menu or combination of meal menus in the user terminal 3. The meal menu recommendation unit 117 can set in advance constraints for combining a plurality of meal menus. For example, restrictions on the number of meal menus included in one combination, an upper limit of the total amount of ingredients included in the meal menu, an upper limit of an intake of one or more nutrients in one meal, and the like can be set as the constraints.

In addition, the meal menu recommendation unit 117 can select and recommend a meal menu that can achieve the target value received by the target value input unit 119. The meal menu recommendation unit 117 may estimate a predicted value of the measured value by applying the content of the nutrient included in the meal menu to the prediction model obtained by analysis of the nutrient and the measured value by the analysis unit 115, determine whether or not the predicted value has achieved the goal (the predicted value is equal to or more than the target value in a case where it is more preferable as the measured value is larger, the predicted value is equal to or less than the target value in a case where it is more preferable as the measured value is smaller, and a difference between the predicted value and the target value is smaller than a predetermined threshold in a case where it is more preferable that the measured value is closer to a specific value), and select a meal menu that has achieved the goal. In the case of the prediction model other than the nutrition model, it is possible to estimate the amount of change in measured value by applying at least one of the (preset or input) life stage of the user, the current date and time or a date and time when the user is scheduled to take the meal menu within the intervention period, and the (preset or input) scheduled amount of activity to be performed in a standard manner.

Further, for a given meal menu, the meal menu recommendation unit 117 can recommend nutrient adjustment (change, addition, or deletion) to improve the measured value. The meal menu recommendation unit 117 performs nutrient adjustment to adjust the degree of intervention (the degree of adjustment) for each nutrient such that the degree of improvement due to the improvement factor exceeds the degree of deterioration due to the deterioration factor. The degree of intervention (the degree of adjustment) is the degree of increase in a case where the degree of improvement due to the improvement factor and the deterioration factor included in the meal (that is, the meal that the user has taken so far) before adjustment by the meal menu recommendation unit 117 is compared with the degree of improvement due to the improvement factor and the deterioration factor included in the meal after adjustment of the content of the nutrient recommended by the meal menu recommendation unit 117.

For example, it is assumed that the target value is higher than the current value of the measured value (that is, if the regression coefficient is positive, it is the improvement factor, and if the regression coefficient is negative, it is the deterioration factor). Here, it is assumed that the regression coefficient×the total content of the improvement factor included in the meal menu before adjustment is 5, and the regression coefficient×the total content of the deterioration factor included in the meal menu before adjustment is −3. Assuming that the regression coefficient×the total content of the improvement factor included in the meal menu after adjustment by the meal menu recommendation unit 117 is 8, and the regression coefficient×the total content of the deterioration factor included in the meal menu after adjustment is −1, the degree of nutrient adjustment (the degree of intervention) is “5”, which is the sum of an adjustment amount (that is, 8−5=3) of the coefficient×the total content relating to the improvement factor and the adjustment amount (that is, (−1)-(−3)=2) of the coefficient×the total content relating to the deterioration factor. It is expected to increase the measurement value as the meal menu recommendation unit 117 performs nutrient adjustment such that the degree of intervention (adjustment degree) becomes positive.

In a case where the target value of the measured value is lower than the current value, the negative regression coefficient becomes the improvement factor, and the positive regression coefficient becomes the deterioration factor, and it is expected to decrease the measurement value (improvement) as the nutrient adjustment is performed such that the amount of change (the degree of intervention and the degree of adjustment) in the regression coefficient×the total content before and after the adjustment becomes negative.

In addition, in a case where the meal menu recommendation unit 117 uses the degree of improvement and the degree of deterioration (the absolute value of the regression coefficient), it is expected that the measurement value is improved (increase or decrease) if a value obtained by subtracting the degree of deterioration×the content of the deterioration factor from the degree of improvement×the content of the improvement factor in the meal menu after adjustment (post-adjustment improvement value) is larger than a value obtained by subtracting the degree of deterioration×the content of the deterioration factor from the degree of improvement×the content of the improvement factor in the meal menu before adjustment (pre-adjustment improvement value), that is, if the degree of intervention (the degree of adjustment) is positive.

The meal menu recommendation unit 117 is only required to perform nutrient adjustment such that the degree of intervention (adjustment degree) becomes a value for improvement (in a case where the regression coefficient is used, the regression coefficient becomes positive to increase the measurement value and becomes negative to decrease the measurement value).

In this way, when the improvement factor and the deterioration factor are summed, the total degree of intervention (the total degree of adjustment) as an index of an overall influence of the adjustment of the improvement factor and the deterioration factor in the intervention period can be obtained, and the total degree of intervention (the total degree of adjustment) always results in the improvement factor >the deterioration factor. The total degree of intervention (the total degree of adjustment) results in the improvement factor >the deterioration factor in three patterns: (1) the positive improvement factor and the positive deterioration factor, (2) the positive improvement factor and the negative deterioration factor, and (3) the negative improvement factor and the negative deterioration factor. If the pattern of the total degree of intervention (the total degree of adjustment) can be achieved, adjustment of an individual nutrient (including the activity amount), whether it is the improvement factor or the deterioration factor, can be freely performed.

In addition, for a given meal menu, the meal menu recommendation unit 117 can recommend adjustment by changing or adding an ingredient (including a seasoning) so as to bring the measured value close to the target value. In the present embodiment, it is assumed that the meal menu given to the meal menu recommendation unit 117 is a meal menu for each of a plurality of meals (preferably all meals) ingested during the intervention period.

It is also possible to give one meal menu for one meal or three meal menus for one day's meal to the meal menu recommendation unit 117. In this case, it is sufficient if the analysis is performed by setting the intervention period to one time or one day.

The meal menu recommendation unit 117 can change, add, and/or delete an ingredient (or a meal menu included in a set of meal menus) included in a meal menu such that the degree of improvement×the content of the improvement factor exceeds the degree of deterioration×the content of the deterioration factor and an excess amount thereof is as large as possible. In addition, the meal menu recommendation unit 117 can change, add, and/or delete an ingredient (or a meal menu included in a set of meal menus) included in a meal menu such that the above-described total degree of intervention (the total degree of adjustment) results in the improvement factor (the adjustment value of the degree of improvement×the content)>the deterioration factor (the adjustment value of the degree of deterioration×the content).

For example, for one or more ingredients A included in the given meal menu, the meal menu recommendation unit 117 can select, from the ingredient information storage unit 136, an ingredient B containing a larger amount of nutrients, which are the improvement factors, than the ingredient A, change the ingredient A to the selected ingredient B, and present the change in the user terminal 3.

The meal menu recommendation unit 117 can estimate the amount of change in measured value by applying the content of the nutrient included in the selected meal menu to the nutrition model, and calculate a first predicted value of the measured value by adding the estimated amount of change to a measurement standard value stored in the measured value storage unit 131, and in a case where the predicted value does not achieve the goal, for one or more ingredients B, the meal menu recommendation unit 117 can change the ingredient A to the ingredient B, calculate a second predicted value of the measured value in the same manner as described above, check whether or not the second predicted value can achieve the goal, and select the ingredient B that can achieve the goal.

In a case where there is no ingredient B that can achieve the goal, the meal menu recommendation unit 117 can select the ingredient B that can achieve the smallest difference between the target value and the predicted value. In this case, the meal menu recommendation unit 117 can output the second predicted value as an achievable measured value.

For an ingredient C included in the given meal menu, the meal menu recommendation unit 117 may select, from the ingredient information storage unit 136, an ingredient D containing a smaller amount of nutrients, which are the deterioration factors, than the ingredient C, change the ingredient C to the selected ingredient D, and present the change in the user terminal 3.

In addition, the meal menu recommendation unit 117 may recommend to delete an ingredient in which the number of nutrients, which are the deterioration factors, is a predetermined number or more, an ingredient or a meal menu in which the content of nutrients, which are the deterioration factors, is a predetermined amount or more, an ingredient or a meal menu in which the number of nutrients, which are the deterioration factors, is larger than the number of nutrients, which are the improvement factors, and an ingredient or a meal menu in which the sum of the absolute values of the regression coefficients of the deterioration factors is larger than the sum of the absolute values of the regression coefficients of the improvement factors.

The meal menu recommendation unit 117 may select an ingredient containing a nutrient, which is the improvement factor, from the ingredient information storage unit 136, add the ingredient to the meal menu, and present the addition in the user terminal 3.

The meal menu recommendation unit 117 may select the meal menu by using the degree of improvement or the degree of deterioration, that is, the absolute value (an improvement coefficient for the improvement factor and a deterioration coefficient for the deterioration factor) of the regression coefficient calculated by the analysis unit 115. The meal menu recommendation unit 117 may calculate a first value obtained by subtracting a value (hereinafter, referred to as a deterioration value) obtained by multiplying the content of the deterioration factor included in the meal menu by the deterioration coefficient relating to the deterioration factor from a value (hereinafter, referred to as an improvement value) obtained by multiplying the content of the improvement factor included in the meal menu by the improvement coefficient relating to the improvement factor, select an ingredient G such that a second value obtained by subtracting the deterioration value relating to the changed meal menu from the improvement value relating to the changed meal menu for the changed meal menu in which at least one ingredient F included in the meal menu is changed to the ingredient G is larger than the first value, change the ingredient F to the selected ingredient G, and present the meal menu in the user terminal 3.

The meal menu recommendation unit 117 may calculate the first value obtained by subtracting the deterioration value relating to the meal menu from the improvement value relating to the meal menu, select an ingredient H such that the second value obtained by subtracting the deterioration value from the improvement value is larger than the first value for the meal menu after adding the additional ingredient H to the meal menu, add the selected ingredient H to the meal menu, and present the addition in the user terminal 3.

The analysis unit 115 may periodically perform analysis to specify the improvement factor and the deterioration factor, and the evaluation unit 116 may evaluate the meal menu according to the analysis performed by the analysis unit 115. For example, the analysis and recommendation may be performed a plurality of times during the intervention period. In this case, it is assumed that the meal menu recommendation unit 117 adjusts the future meal menu (and activity amount) without changing the ingredients (nutrients), the activity amount, and the like for the past meal menu.

The activity recommendation unit 118 can present the activity amount for achieving the goal in the user terminal 3.

For example, when the target value cannot be achieved only with the meal menu recommended by the meal menu recommendation unit 117, the activity recommendation unit 118 can recommend the activity. The activity recommendation unit 118 can calculate the activity amount estimated to be able to achieve the target value for the activity amount of the recommended action. For example, it is possible to calculate, from the meal menu (with a changed, added, or deleted ingredient) recommended by the meal menu recommendation unit 117 on the basis of the nutrition model, the first predicted value of the measurement value achieved in a case where the activity amount is 0 (which can be calculated by applying the content of the nutrient included in the meal menu to the nutrition model). Therefore, in a case where the first predicted value cannot achieve the target value, adjustment of the activity standard value can be recommended. In this case, the analysis unit 115 estimates the nutrition-activity model, and the meal menu recommendation unit 117 can calculate the first predicted value by applying the activity standard value and the content of the nutrient to the nutrition-activity model, and recommend a meal menu in which an ingredient has been changed, added, and/or deleted to bring the first predicted value closest to the target value. Then, the activity recommendation unit 118 can adjust the activity amount according to the improvement coefficient or the deterioration coefficient relating to the activity. The activity recommendation unit 118 can perform activity amount adjustment by changing the activity amount such that the second predicted value calculated by applying the activity amount and the content of nutrient included in the meal menu recommended by the meal menu recommendation unit 117 to the nutritional-activity model reaches the target value.

Furthermore, in a case where the predicted value estimated from the nutrition-activity model has not reached the target value, and the activity is the improvement factor, for example, the activity recommendation unit 118 can calculate an increase amount of the activity amount corresponding to a shortage by, for example, dividing the shortage by the improvement coefficient, calculate the activity amount by adding the calculated amount to the activity standard value, and transmit, to the user terminal 3, a message for recommending the activity together with the calculated activity amount. Conversely, in a case where the activity is the deterioration factor, the activity recommendation unit 118 can calculate a decrease amount (it is assumed that the activity standard value is the maximum value) of the activity amount corresponding to a shortage by dividing the shortage by the deterioration coefficient or the like, and transmit a message recommending not to perform the activity together with the calculated decrease amount to the user terminal 3.

The activity recommendation unit 118 can also propose an activity amount that improves the measured value by using the regression coefficient (the improvement coefficient or the deterioration coefficient) relating to the activity amount in the nutritional-activity model calculated by the analysis unit 115. For example, the activity recommendation unit 118 calculates a value (hereinafter, referred to as an activity amount value) obtained by multiplying the activity amount analyzed by the analysis unit 115 by the regression coefficient. In a case where the activity amount is the improvement factor, the larger the activity amount value is, the more the measured value is improved. In a case where the activity amount is the deterioration factor, the smaller the activity amount value is, the more the measured value is improved. In a case where the activity amount is the improvement factor, the activity recommendation unit 118 is only required to calculate the activity amount that is recommended for the age group or gender of the user or activity amount that is not excessively large obtained by multiplying an average value of the past activity amounts of the user by a predetermined value, and can present the activity amounts in the user terminal 3. Furthermore, in a case where the activity amount is the deterioration factor, the activity recommendation unit 118 may recommend the activity amount of 0 so that the activity amount value is minimized. However, for an activity that causes health/life problems when the activity amount is 0 (for example, walking, but the present invention is not limited thereto), it is only required to calculate the activity amount of the activity recommended to be performed at the minimum for the age group or gender of the user, or the activity amount that is not excessively small obtained by multiplying the average value of the past activity amounts of the user by a predetermined value, and the activity amounts can be presented in the user terminal 3.

In addition, the meal menu recommendation unit 117 can recommend the meal menu and the activity after adjusting the activity amount together with adjusting the nutrient (ingredient) of the given meal menu.

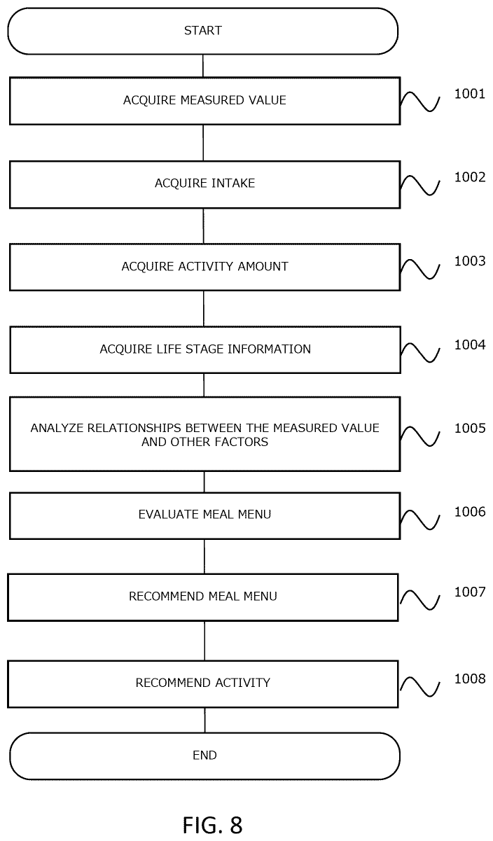

A typical processing flow of the server device 1 will be described with reference to FIG. 8. The measured value acquisition unit 111 acquires the measured value from the user terminal 3 (1001). The intake acquisition unit 112 acquires the intake from the user terminal 4 (1002). The activity amount acquisition unit 113 acquires the activity amount from the user terminal 3 (1003). The life stage information acquisition unit 114 acquires the life stage information from the user terminal 3 (1004). The analysis unit 115 analyzes the relationships between the measured value and other factors such as the intake, the activity amount, and the life stage information (1005). The evaluation unit 116 evaluates the meal menu on the basis of the analysis result of the analysis unit 115 (1006). The meal menu recommendation unit 117 presents the meal menu selected on the basis of the evaluation result of the evaluation unit 116 in the user terminal 3. (1007). The activity recommendation unit 118 presents the activity selected on the basis of the evaluation result of the evaluation unit 116 in the user terminal 3. (1008).

Although the preferred embodiments of the present disclosure have been described in detail with reference to the accompanying drawings, the technical scope of the present disclosure is not limited to such examples. It is obvious that a person having ordinary knowledge in the technical field of the present disclosure can conceive various changes or modifications within the scope of the technical idea described in the claims, and it is naturally understood that these also belong to the technical scope of the present disclosure.

For example, the meal menu recommendation unit 117 may select the meal menu in consideration of information regarding a recipe or a process of the meal menu stored in the meal menu storage unit 135 and information regarding an influence of cooking of each ingredient on a nutrient and an influence of simultaneous cooking with other ingredients on a nutrient stored in the ingredient information storage unit 136. In this case, in a case where an ingredient is added to the meal menu, it is sufficient if the meal menu recommendation unit 117 evaluates a relationship between the process of the meal menu and the added ingredient, and calculates, in a case where the nutrient of the added ingredient or the originally included ingredient varies, the nutrient after the variation as the amount of the improvement factor or the deterioration factor. In addition, in a case where an ingredient is added to the meal menu, it is sufficient if the meal menu recommendation unit 117 evaluates a relationship between the ingredient included in the meal menu and the added ingredient, and calculates, in a case where the nutrient of the added ingredient or the originally included ingredient varies, the nutrient after the variation as the amount of the improvement factor or the deterioration factor.

In the present embodiment, the regression coefficient is used as it is as the improvement coefficient and the deterioration coefficient, but the present invention is not limited thereto, and the degree of intervention (the degree of adjustment) may be calculated using a numerical value obtained by multiplying a difference from a standardized nutrient content by a standardized coefficient (obtained by standardizing the coefficient of the explanatory name variable in the multiple regression formula). In this case, the degree of intervention (the degree of adjustment) in the case of adjusting the content of nutrient becomes a difference between the content of the nutrient included in the meal menu before the change and the content of the nutrient included in the meal menu after the change. In this case, the degree of intervention (the degree of adjustment) can be calculated using a numerical value obtained by multiplying the difference from the content of the nutrient that is not standardized (the coefficient itself of the explanatory name variable in the multiple regression formula). Then, if the pattern of the total degree of intervention (the total degree of adjustment) is the improvement factor >the deterioration factor, the meal menu recommendation unit 117 can perform nutrient adjustment to achieve the target value (approach the target value).

The device described in the present specification may be implemented by a single device, or may be implemented by a plurality of devices (for example, a cloud server) or the like partially or entirely connected by a network. For example, each functional unit and each storage unit of the server device 1 may be implemented by different servers connected to each other via a network.

The series of processing in the device described in the present specification may be implemented by using any of software, hardware, and a combination of software and hardware. A computer program for implementing each function of the server device 1 according to the present embodiment can be produced and mounted on a personal computer (PC) or the like. Furthermore, a computer-readable recording medium storing such a computer program can also be provided. The recording medium is, for example, a magnetic disk, an optical disk, a magneto-optical disk, or a flash memory. Furthermore, the computer program described above may be distributed via, for example, a network without using a recording medium.

Furthermore, the processing described with reference to the flowchart in the present specification does not have to necessarily be executed in the illustrated order. Some processing steps may be performed in parallel. In addition, additional processing steps may be employed, and some processing steps may be omitted.

Furthermore, the effects described in the present specification are merely illustrative or exemplary, and are not restrictive. That is, the technology according to the present disclosure can exhibit other effects obvious to those skilled in the art from the description of the present specification together with or instead of the above effects.

Hereinafter, examples of the health support system according to the present embodiment will be described.

### First Example

In a first example, the body weight was selected as a health index to be achieved by optimizing an intake nutrient amount. A relationship between the body weight and the intake nutrient amount, and the amount of exercise (step count) was clarified by analysis, and whether or not a body weight improvement effect by an optimal personal diet with an optimal nutrient amount can be obtained was verified by conducting a test for humans, and an effective analysis method for nutrient optimization was examined.

### Test Method

### Used Equipment and Software

Measurement/recording equipment and software including a mobile application used is described below. In a case where pieces of equipment used in a feasibility test and equipment used in a clinical test are different from each other, symbols are marked on the items. (+) indicates that the equipment was used only for the feasibility test, and (*) indicates that the equipment was used only for the clinical test.

### Used Equipment and Reagent

1) Body weight and composition monitor (HBF-255T, Omron Healthcare Co., Ltd., Kyoto)
2) Activity meter (*) (Fitbit Charge3, Fitbit (Google), USA)
3) Smartphone (*) (iPhone (registered trademark) SE (2020), Apple, USA)
4) Blood test kit (+) (DEMECAL blood test kit [metabolic syndrome & lifestyle disease self-check], Leisure, Inc., Tokyo)

### Used Software

1) OMRON connect (+) (Omron Healthcare Co., Ltd., Kyoto)
2) Asken Diet (+) (asken Inc., Tokyo)
3) Fitbit (+) (Fitbit (Google), USA)
4) Mr. KenDateman Web (*) (Chi-technology Co., Ltd., Tokyo)
5) SPSS Statistics ver. 25 (IBM, USA)
6) SPSS Statistics ver. 28(+)(IBM, USA)

### Personal Optimization of Intake Nutrient Amount

### Derivation of Body Weight Prediction Model Formula

In the first example, a “body weight prediction model formula” for predicting the body weight of a test subject from a factor such as a nutrient was created. The body weight prediction model formula predicts a daily wake-up weight acquired for each test subject from each data such as an intake nutrient amount (selected from 21 components including water, protein, lipid, sugar, a total dietary fiber content, carbohydrate, sodium, potassium, calcium, magnesium, iron, zinc, vitamin A, vitamin D, vitamin E, vitamin B1, vitamin B2, vitamin B6, vitamin C, salt equivalent, and saturated fatty acid), an intake energy amount, and the amount of exercise (step count). In deriving the model formula, multiple regression analysis, which is an analysis method for predicting a certain objective variable from a relationship with a plurality of other variables (explanatory variables) using only the explanatory variables, was adopted. SPSS Statistics ver. 25 and ver. 28 (IBM, USA) were used as software for performing multiple regression analysis, and multiple regression analysis by a backward elimination method was performed with the objective variable as the wake-up weight and the explanatory variable as the intake nutrient amount/the amount of exercise one day before to three days before.

It is known that when there is a strong correlation between the selected explanatory variables, there is a high possibility that the model formula cannot be calculated correctly and a prediction error increases. In general, the number of pieces of data required in the multiple regression analysis is 10 to 15 times the number of pieces of data used as the explanatory variables, but in the case of application to the first example, a required period exceeds one year, which is not realistic, and cannot be adapted to seasonal changes and physical growth. For this reason, a data collection period is one month as a standard, and a model formula (Formula 1), in which the number of selected explanatory variables is seven or less, which is ¼ of the number of explanatory variables, and a variance inflation factor (VIF) of the explanatory variables is 10 or less, which can exclude a possibility of multicollinearity in which a correlation coefficient is inappropriately higher than a true value due to the correlation between the variables, is adopted. The intake nutrient (explanatory variable) selected here is a variable selected for prediction of the wake-up weight, and is considered to be a weight-related nutrient that affects the body weight. In the formula, a nutrient with a positive sign for the multiple regression coefficient has a positive correlation with the body weight, and thus is a weight gain factor considered to increase the body weight when ingested. On the other hand, a nutrient with a negative sign for the multiple regression coefficient has a negative correlation with the body weight, and thus is a weight loss factor considered to decrease the body weight when ingested.

$$Body Weight Prediction Model Formula$$

$$Wake - up weight = Nutrient 1 \times Coefficient 1 + Nutrient 2 \times Coefficient 2 … + Constant term \qquad ( Formula 1 )$$

### Optimization of Nutrient Amount

In individually designing a test food that is expected to improve the body weight of the test subject, adjustment of the amount of food including nutrients that work for weight gain and weight loss was performed for the test subject determined by the body weight prediction model formula.

When the total value of nutrients included in a daily meal ingested by the test subject was substituted into the model formula, the amount of nutrients ingested by the test subject in a day was optimized by changing ingredients to be used in a menu of the test food or performing adjustment mainly by changing a serving amount of a menu such that a predicted weight approaches a value indicating 21.5 to 24.9 kg/m², which is a target BMI range.

However, in order to avoid health impairment due to excess or deficiency of nutrients, the following conditions (1) to (4) were defined, and Conditions (2) and (4) were followed in the feasibility test, and all of Conditions (1) to (4) were followed in the clinical test. A content of the menu of the test food varied for each meal and was not consecutive, and a psychological stress of the test subject due to continuation of the same menu was avoided.

### “Conditions” (1)

Estimated average required amounts and target amounts of three major nutrients (carbohydrates, fat, and proteins) defined in the Dietary Reference Intakes for Japanese (2020 edition) are amounts that are more than or equal to the minimum value and less than 130% of the maximum value in the age and gender of the test target.
(2) One that does not exceed it for nutrients with a tolerable upper limit.
(3) The specified amount of weight-related component in the observation period is 70% or more of the estimated average required amount or the standard amount in the Dietary Reference Intakes for Japanese.
(4) The amount of energy ingested per week is the same as a daily diet of the test target.

### Test Food and Intake Nutrient Amount

The test food is prepared in the same manner as when ordinary people prepare ordinary meals using commercially available ingredients. In the feasibility test, the test target himself or herself purchased designated foods according to a menu table in which ingredients and use amounts are designated by optimizing the nutrient amount at a supermarket or the like, and cooked.

In the clinical test, the amount of nutrients was optimized by adjusting or replacing the amount of ingredients based on a basic menu created under the supervision of an administrative dietitian in consideration of a balance between a main dish and a side dish, a “preparation table” in which the ingredients and the use amounts are designated was delivered to a test site, and the meal was cooked according to the “preparation table” and served individually to the test subject. The meal was based on three meals a day of breakfast, lunch, and dinner, and snacks (10:00, 15:00, and late-night snack) were added in accordance with an eating habit so far. In addition, when the test subject did not finish the specified amount of test food, the remaining amount was recorded, and a complete eating rate was calculated from the serving amount to calculate the intake.

### Feasibility Test

### Test Target Characteristics

The test target of the conducted feasibility test was one healthy adult male who was 25 years old as of April 2021. The height was 172.1 cm, and this value was used for calculating the BMI (kg/m²).

### Test Schedule

The feasibility test was conducted twice in total for 98 days from Sep. 1, 2020 to Dec. 7, 2020 and for 63 days from Oct. 26, 2021 to Dec. 27, 2021.

### Test in 2020

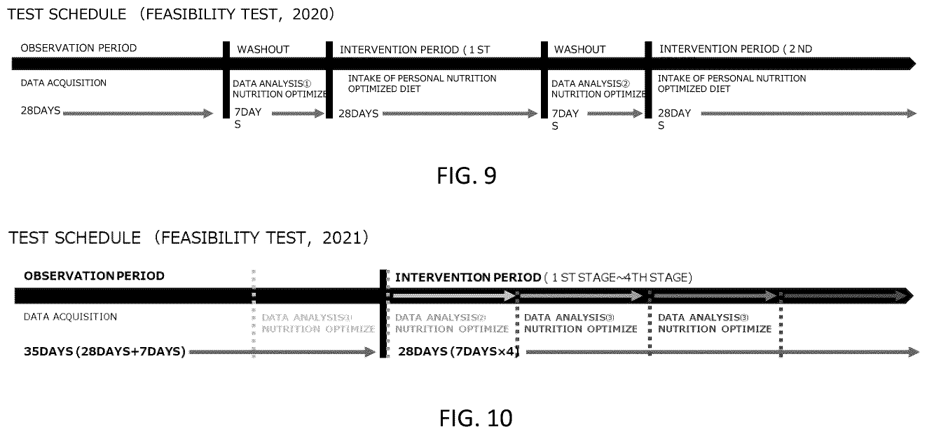

The test was conducted based on a schedule (FIG. 9) including four periods: the observation period (28 days), a washout period (7 days×2), the intervention period (28 days×2), and a follow-up period (14 days).

In the observation period, the body weight and composition were measured and recorded at the time of wake-up and before going to bed, and the type and amount of the ingredient used for the meal were recorded. In the washout period, in consideration of a possibility that the body weight is improved due to a behavior modification such as improvement of a lifestyle of the test subject by data acquisition in the observation period, the test subject returned to the normal lifestyle without performing any data measurement, and test subject data analysis and preparation of the test food in which the nutrients were optimized based on the analysis result were performed. In the intervention period, the test subject was caused to ingest the test food subjected to nutrient adjustment as breakfast, lunch, and dinner basically as in the observation period, and the body weight and composition were measured as in the observation period.

### Test in 2021

The clinical test was performed based on a schedule (FIG. 10) roughly divided into two periods of the observation period (35 days) and the intervention period (28 days). Changes from the test conducted in 2020 are that daily data measurement was continued without interruption by eliminating the washout period during which data acquisition is not performed, and that the intervention period was divided into first to fourth stages and multiple regression analysis was performed every week in consideration of a possibility that an environment in the body changes from moment to moment and a nutrient that is the weight-related factor changes to update the weight prediction model formula used for nutrient optimization.

### Measurement of Body Weight and Composition Data

For measurement of each data such as the body weight and composition (a body fat percentage (%), a skeletal muscle percentage (%), and a basal metabolic rate (kcal)), measurement was performed three times at the time of wake-up using a household body weight and composition monitor. When processing each data, an average value of values obtained by performing the measurement three times was calculated and used.

In addition, the amount of exercise (step count) was measured by wearing a wristwatch-type activity meter on the left wrist throughout a day except for a time in which the activity meter is removed to charge a main body of the activity meter at the time of bathing.

### Estimation of Dietary Intake Nutrient Amount

During the observation period, meals were registered at breakfast, lunch, and dinner using a smartphone application for meal management. Using a meal recording function of the application, the intake of a total of 20 components of protein, lipid, sugar, a total dietary fiber content, carbohydrate, sodium, potassium, calcium, magnesium, iron, zinc, vitamin A, vitamin D, vitamin E, vitamin B1, vitamin B2, vitamin B6, vitamin C, salt equivalent, and saturated fatty acid, and the intake energy amount were estimated from the ingested meal, and the total amount per day was recorded for creating the model formula for predicting the body weight from a daily nutrient amount. The intake energy amount was used to set a range in which an energy amount of the meal menu with the optimized nutrient amount is unchanged from the observation period.

Data conforming to the Standard Tables of Food Composition in Japan (seventh edition, 2015) was used in the test conducted in 2020, and data conforming to the Standard Tables of Food Composition in Japan (eighth edition, 2020) revised in December 2020 was used in the test conducted in 2021.

### Blood Test

In the feasibility test conducted in 2021, a blood test using a blood test kit was conducted in order to examine the environment in the body influenced by dietary life during the observation period and the intervention period. Measurement points were a total of two time points after the end of the observation period and after the end of the intervention period, and blood was collected before breakfast after wake-up. A blood sample obtained by blood collection was sent to a laboratory facility and examined. The amount of blood collected was 0.065 mL per one time.

### Clinical Test

### Test Subject Characteristics

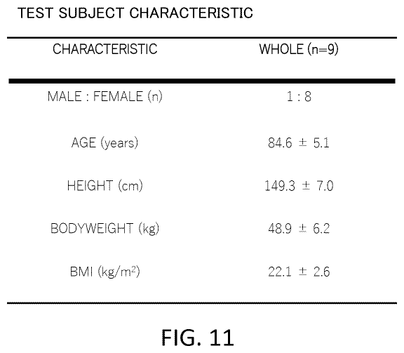

In the clinical test, a facility staff member, a test subject candidate, and a legal representative (a person who has parental authority, spouse, guardian, or equivalent person for the test subject) of the test subject candidate who is a dementia patient were explained according to “Ethical Guidelines for Medical and Health Research Involving Human Subjects”, and a total of nine individuals who finally provided consent forms were selected as the test subjects for the clinical test. The test subject characteristics are as illustrated in FIG. 11.

### Test Schedule

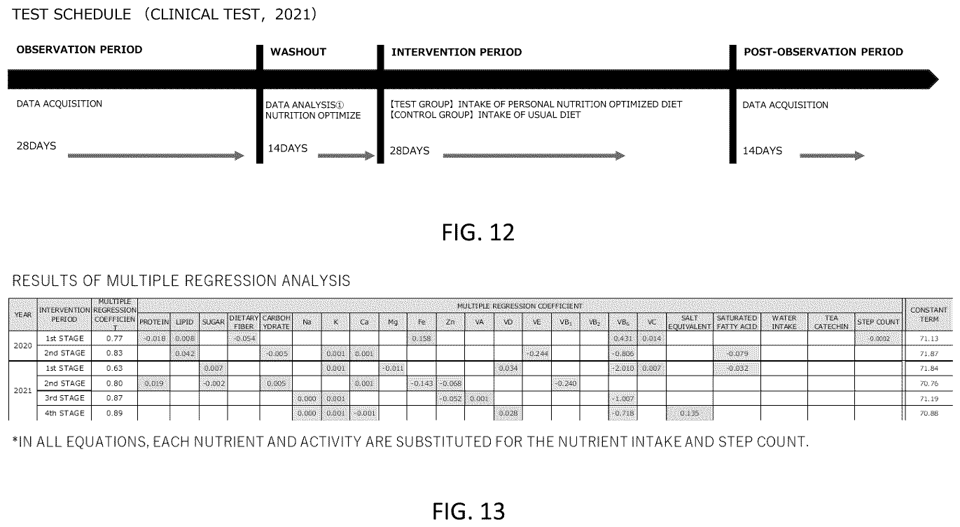

In the clinical test, screening by the BMI calculated from the weight and height obtained by preliminary measurement was performed to select and group the test subjects, and then the clinical test was performed based on a schedule including four periods of the observation period (28 days), the washout period (14 days), the intervention period (28 days), and the follow-up period (14 days) (FIG. 12).

In the observation period, the body weight and composition were measured at the time of wake-up and before going to bed, and a use amount of ingredients and the serving amount and the remaining amount of food for calculating a dietary intake rate of the test subject were recorded in a menu table provided in advance.

In the washout period, in consideration of a possibility that the body weight is improved due to a behavior modification such as improvement of a lifestyle of the test subject by data acquisition in the observation period, the test subject returned to the normal lifestyle without performing any data measurement, and test subject data analysis and preparation of the test food in which the nutrients were optimized based on the analysis result were performed.

In the intervention period, each test subject was caused to ingest the test food subjected to nutrient adjustment as breakfast, lunch, dinner, and snacks (10:00, 15:00, and a late-night snack) basically as in the observation period, the body weight and composition were measured similarly to the observation period, and the use amount of the ingredients, the serving amount for the test subject, and the remaining amount were recorded.

In the follow-up period, similarly to the observation period, the body weight and composition were measured at the time of wake-up and before going to bed, and in addition, the use amount of the ingredients, the serving amount for the test subject, and the remaining amount were recorded.

The clinical test was conducted with the approval of the Ethics Review Board. In addition, an explanatory meeting for informed consent was held for the test subjects, and the test subject's consent was obtained.

### Test Subject Screening

The body weight and composition monitor and body weight recording paper were distributed, the body weight was measured (at the time of wake-up and before going to bed) for three days, and screening was performed according to the result. The BMI was calculated based on the body weight measurement result and the height, and the test subjects were divided into two groups of a test group and a control group based on the following selection criteria.

### “Test Group Selection Criteria”

(1) A person who received prior explanation, understood the content, agreed with the purpose, and provided written consent either personally or through a legal representative
(2) A person whose BMI value calculated based on the body weight measured for three days is outside the following target body weight range
(3) A target body weight (over 65 years old): BMI 21.5 to 24.9 kg/m²
(4) A person who regularly eat breakfast, lunch, and dinner and take a balanced meal including staple foods, main dishes, and side dishes without picky eating habits
(5) A person who can take the cooked test food according to an instructed menu and a serving instruction.
(6) A person who does not regularly consume alcoholic beverages and can refrain from consuming alcoholic beverages during the test period

### “Control Group Selection Criteria”

(1) Among persons satisfying (1), (2), and (4) to (8) of the “test group selection criteria”, a person whose BMI value calculated based on the body weight measured for three days is within the following target body weight range (target body weight (over 65 years old): BMI 21.5 to 24.9 kg/m²)

### “Exclusion Criteria”

(1) A person exhibiting significant abnormalities in body weight measurement
(2) A person who has undergone abdominal surgery within six months before a test start date
(3) A woman experiencing significant changes in physical condition before and after menopause
(4) A person at risk of allergic reactions to drugs or food
(5) A woman who is pregnant or might be pregnant and a women who is breastfeeding
(6) A person who is currently participating in other clinical test or who has participated within the past four weeks
(7) A person undergoing continuous treatment by blood donation and intravenous, intramuscular, or subcutaneous injections
(8) Others determined to be ineligible by the study representative

### Measurement of Body Weight and Composition Data

For measurement of each data such as the body weight and composition (a body fat percentage (%), a skeletal muscle percentage (%), and a basal metabolic rate (kcal)), measurement was performed three times at the time of wake-up and before going to bed using a body weight and composition monitor. For data processing, an average value of values obtained by performing the measurement three times was calculated and used.

Body weight and composition data were recorded on recording paper by a person who assists measurement of the test subject. When an error of 0.15 kg or more occurred in the measured value, the fourth measurement was performed, and three average values close to each other were calculated and used at the time of data processing.

### Estimation of Dietary Intake Nutrient Amount

Nutritional management software was used to estimate a dietary intake nutrient amount of the test subject.

The energy amount and the intake of the total of 21 components of water, protein, lipid, sugar, a total dietary fiber content, carbohydrate, sodium, potassium, calcium, magnesium, iron, zinc, vitamin A, vitamin D, vitamin E, vitamin B₁, vitamin B₂, vitamin B₆, vitamin C, salt equivalent, and saturated fatty acid were estimated from the meal ingested in one day by a feeding nutrition amount table output by the nutrition management software. Here, the feeding nutrient amount table is a list of daily intake nutrient amounts for one test subject. In the nutrition management software, data based on the Standard Tables of Food Composition in Japan (eighth edition, 2020) is used.

### Statistical Processing

### Evaluation of Degree of Intervention of Weight-Related Nutrient

In order to evaluate the degree of intervention of the weight-related factor in a weight improvement effect during the intervention period, an index called the degree of intervention was newly provided. The degree of intervention of each weight-related nutrient was obtained by multiplying a value obtained by subtracting an average value of the weight-related factor after standardization of the intervention period from an average value of the weight-related factor after the standardization of the observation period, by an absolute value of a standardization coefficient derived from multiple regression analysis was multiplied (Formula 2).

$$Intervention Degree Calculation Formula$$

$$Degree of intervention = [ Standardized average value of weight - related factor ( intervention period ) - Standardized average value of weight - related factor ( observation period ) ] \times \vert Standardization coefficient \vert \qquad ( Formula 2 )$$

Here, the standardization is one of statistical methods for correcting values such that an average of data becomes 0 and a variance become 1, and enables comparison between weight-related factors such as nutrients having different units. In addition, the standardization coefficient is a value used when the multiple regression coefficient derived by the multiple regression analysis is compared between the explanatory variables having different units, and the two types of values were used in the calculation of the degree of intervention.

The degree of intervention individually calculated for one test subject by Formula 2 was evaluated as follows: (1) when a body weight deterioration factor was a negative value and a body weight improvement factor was a positive value, or (2) when the degree of intervention of the body weight improvement factor exceeded the degree of intervention of the body weight deterioration factor, nutrient adjustment expected to improve the body weight was performed as compared with the observation period.

### Test Method

For the measurement results, an average value and a standard error were calculated for each observation period and intervention period, respectively. Since a measured body weight value on the first day after the start of the intervention was a result of not ingesting the test food, statistical processing was performed by excluding data on the first day of the intervention period during the test. For the measured body weight value of each test subject, an unpaired t-test was selected for comparison between the average values of the observation period and the intervention period, and statistical processing was performed accordingly. A significance level in the test was 5% or less in a two-sided test, and a p value of 0.05 or less was indicated by “*”, a p value of 0.01 or less was indicated by “**”, and a p value of 0.001 or less was indicated by “***”

### Experimental Result

### Feasibility Test Result

### Body Weight Prediction Model Formula

The multiple regression analysis was performed using SPSS Statistics ver. 25 and SPSS Statistics ver. 28 with the wake-up weight acquired in the observation period as an objective variable and the intake nutrient amount and the amount of exercise (step count) as explanatory variables (FIG. 13). Based on the obtained multiple regression coefficient, a model formula for predicting the wake-up weight was constructed, and the weight-related factor was specified from the sign of the coefficient. An average value of the multiple correlation coefficients of a total of six model formulas used in two feasibility tests was 0.80, and a high correlation was observed between the intake nutrient and the measured body weight value, suggesting that the wake-up weight can be predicted from the intake nutrient amount and the amount of exercise (step count).

$$First Stage in Intervention Period in 2020$$

$$Wake - up weight = - 0.018 \times protein + 0.0079 \times lipid - 0.054 \times dietary fiber - 0.16 \times iron + 0.43 \times vitamin {B}_{6} + 0.0 \; 14 \times vitamin C - 0.00017 \times the amount of exercise ( step count ) + 71.13 \qquad ( Formula 3 )$$

$$Second Stage in Intervention Period in 2020$$

$$Wake - up weight = 0.042 \times lipid - 0.0054 \times carbohydrate + 0.00059 \times potassium + 0.0015 \times calcium - 0.24 \times vitamin E - 0.81 \times vitamin {B}_{6} - 0.0 \; 79 \times saturated fatty acid + 71.87 \qquad ( Formula 4 )$$

$$First Stage in Intervention Period in 2021$$

$$Wake - up weight = 0.0065 \times sugar + 0.0014 \times potassium - 0.011 \times magnesium + 0.034 \times vitamin D - 2.01 \times vitamin {B}_{6} + 0.0 \; 070 \times vitamin C - 0.032 \times saturated fatty acid + 71.84 \qquad ( Formula 5 )$$

$$Second Stage in Intervention Period in 2021$$

$$Wake - up weight = 0.019 \times protein - 0.0024 \times sugar + 0.0054 \times carbohydrate + 0.0014 \times calcium - 0.14 \times iron - 0.068 \times zinc - 0.24 \times vitamin {B}_{1} + 70.76 \qquad ( Formula 6 )$$

$$Third Stage in Intervention Period in 2021$$

$$Wake - up weight = - 0.000022 \times sodium + 0.0011 \times potassium - 0.0052 \times zinc + 0.00076 \times vitamin A - 1.01 \times vitamin {B}_{6} + 71.19 \qquad ( Formula 7 )$$

$$Fourth Stage in Intervention Period in 2021$$

$$Wake - up weight = 0.00036 \times sodium + 0.00092 \times potassium - 0.00093 \times calcium + 0.028 \times vitamin D - 0.72 \times vitamin {B}_{6} + 0.14 \times salt equivalent + 70.88 \qquad ( Formula 8 )$$

### Weight-Related Nutrient Amount of Test Food

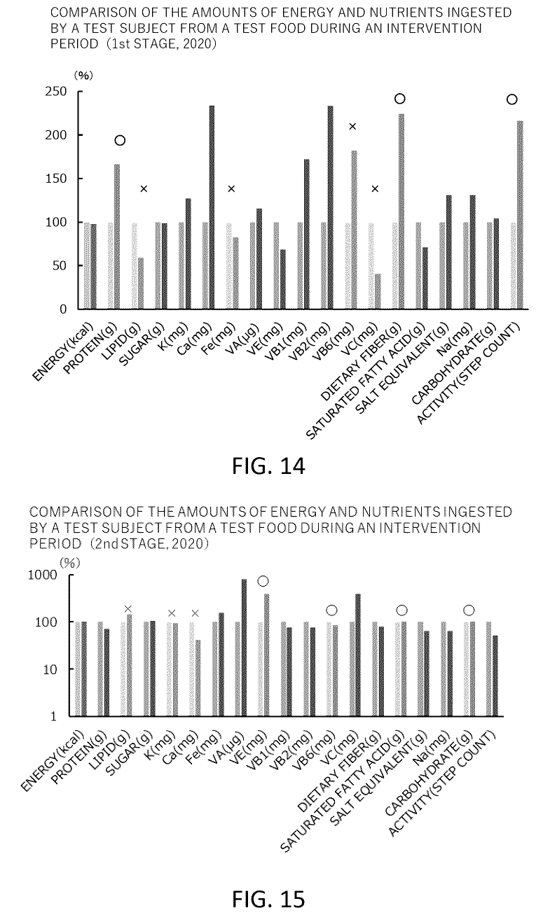

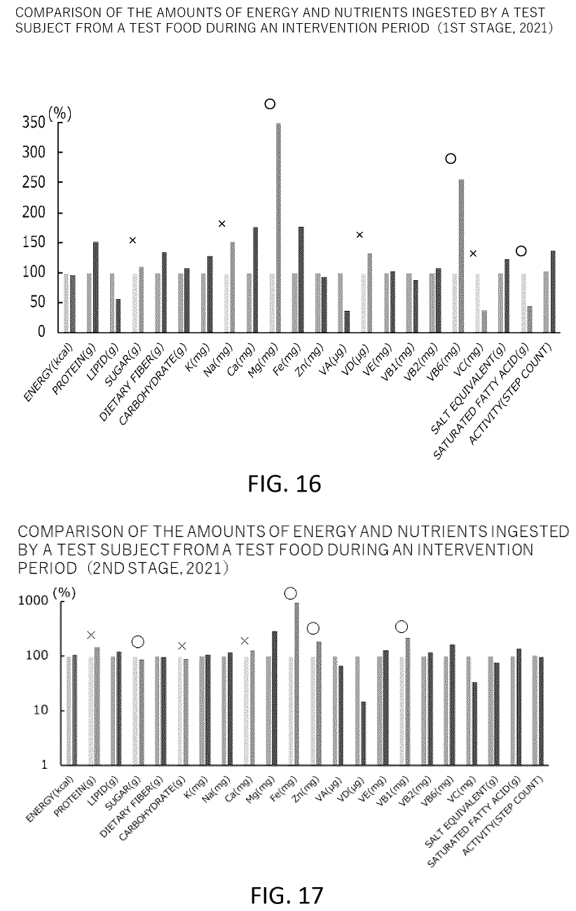

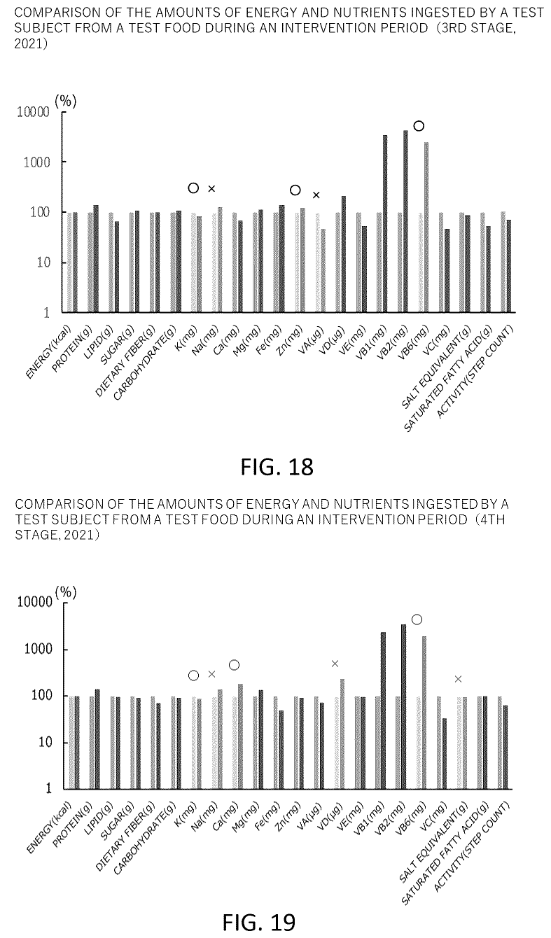

Graphs illustrating comparison of the amounts of energy and nutrients ingested by the test subject from the test food during the intervention period with the intakes during the observation period as 100% are illustrated in FIGS. 14 to 19 below. In two bar graphs with the same label, the left bar graph shows the intake during the observation period and the right bar graph shows the intake during the intervention period. A circle mark indicates the body weight improvement factor, and a cross mark indicates the body weight deterioration factor. In addition, in designing a test food menu, it was determined that the amount of energy is not to be changed, and thus the energy amount was also compared similarly to the nutrients. The amount of energy in the intervention period has achieved adjustment in a range of 95% to 106% compared to the observation period. In addition, in the adjustment of the amount of nutrients, it was possible to perform adjustment to increase the body weight improvement factor and decrease the body weight deterioration factor, with some exceptions. In addition, in the feasibility test conducted in 2020, the adjustment was performed so as not to change a sugar mass from that in the observation period.

### Body Weight Measurement Result

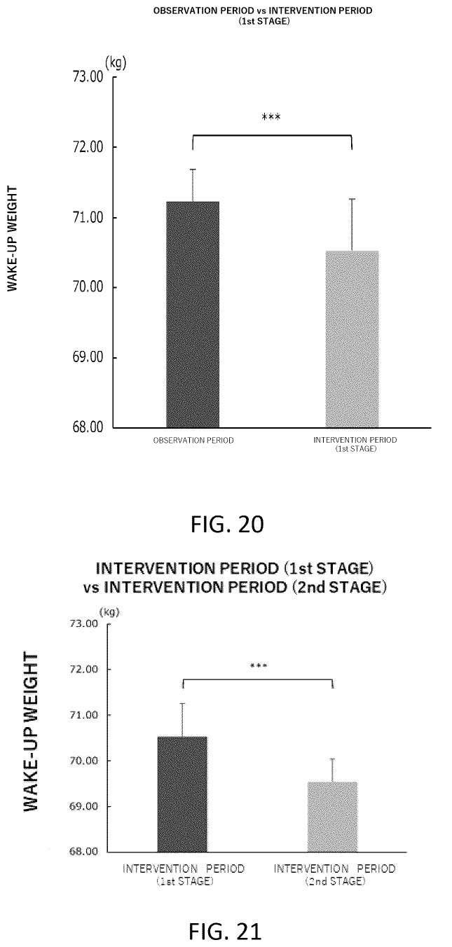

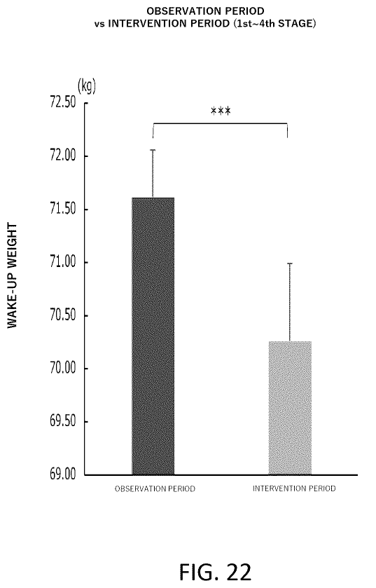

Regarding the wake-up weight measured by the body weight and composition monitor, graphs illustrating comparison of the average values of the observation period and the intervention period are illustrated in FIGS. 20 to 22 below. In comparison of both the measurement periods of the feasibility test conducted in 2020 and the feasibility test conducted in 2021, a significant (p<0.001) weight loss (weight improvement) was confirmed as compared with the average body weight before the intake of the test food and during the observation period.

### Body Composition Measurement Result

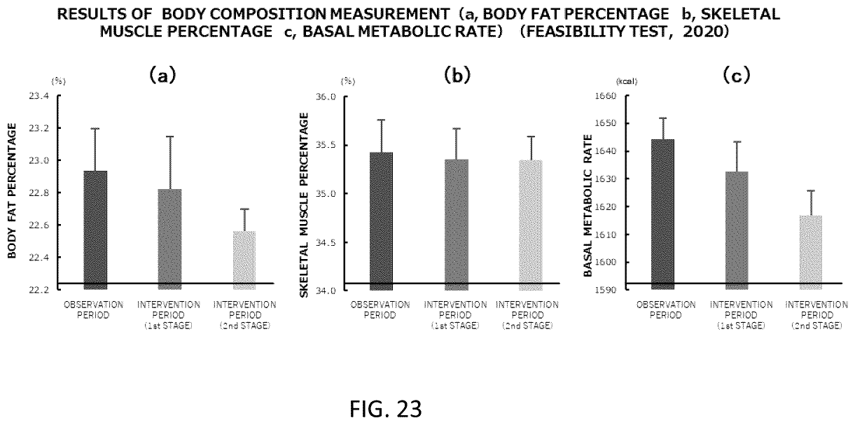

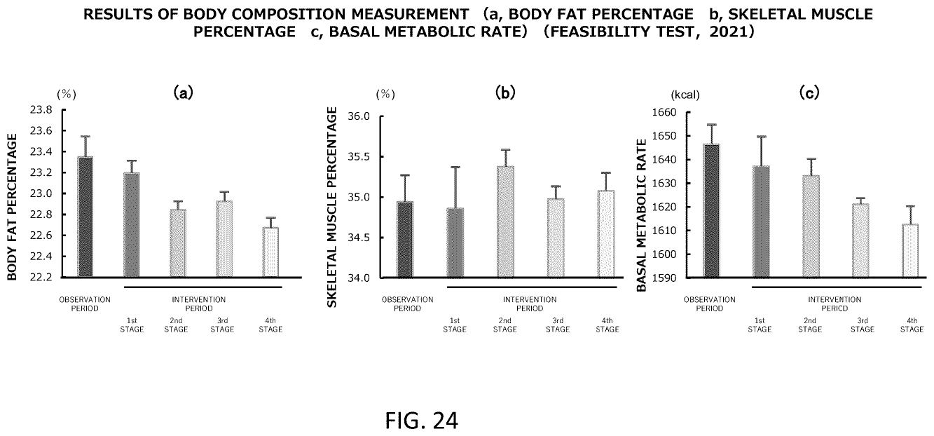

The body fat percentage, the skeletal muscle percentage, and the basal metabolic rate measured by the body weight and composition monitor are illustrated in FIGS. 23 and 24 below. In the measurement of the body composition, it was confirmed that both the body fat percentage and the basal metabolic rate tended to monotonically decrease in both the feasibility test conducted in 2020 and the feasibility test conducted in 2021 (FIGS. 23(a) and 24(a)). As for the skeletal muscle percentage, no change tendency was observed throughout the test period, and therefore it was determined that the body weight improvement (body weight reduction) in the feasibility test was caused by a decrease in body fat.

### Activity Amount Measurement Result

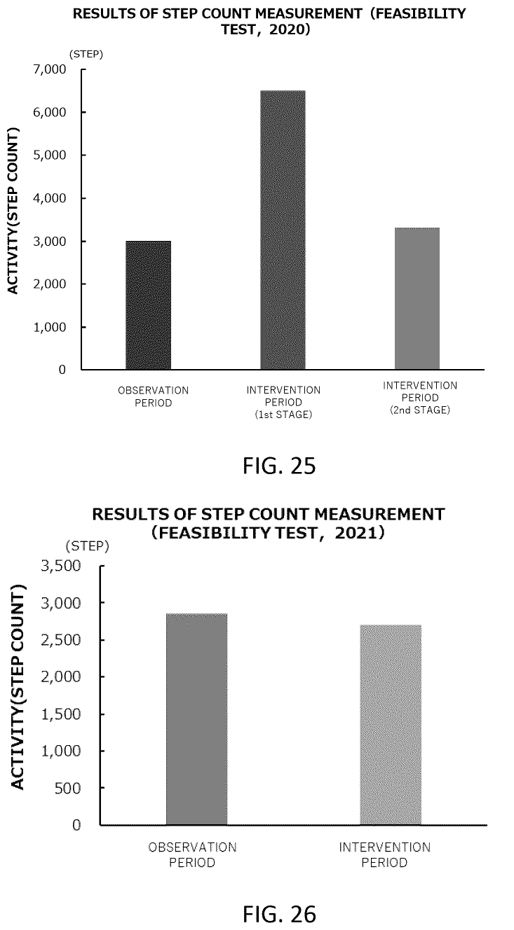

Measurement results of the amount of exercise (step count) measured by the activity meter are illustrated in FIGS. 25 and 26 below.

In the feasibility test conducted in 2020, since the amount of exercise (step count) was incorporated into the model formula as the body weight improvement factor in the intervention period (first stage), a target step count increased from that in the observation period was set (about 200% of that in the observation period) within a reasonable range, and conscious walking was performed such that the total step count in one day reaches the target step count. However, since the amount of exercise (step count) was not incorporated into the model formula as the weight-related factor in other periods, conscious walking was not performed, and a normal life was performed. In the feasibility test conducted in 2021, since the amount of exercise (step count) was not incorporated into the model formula as the weight-related factor, conscious walking was not performed, and a normal life was maintained.

### Evaluation of Degree of Intervention of Nutrient

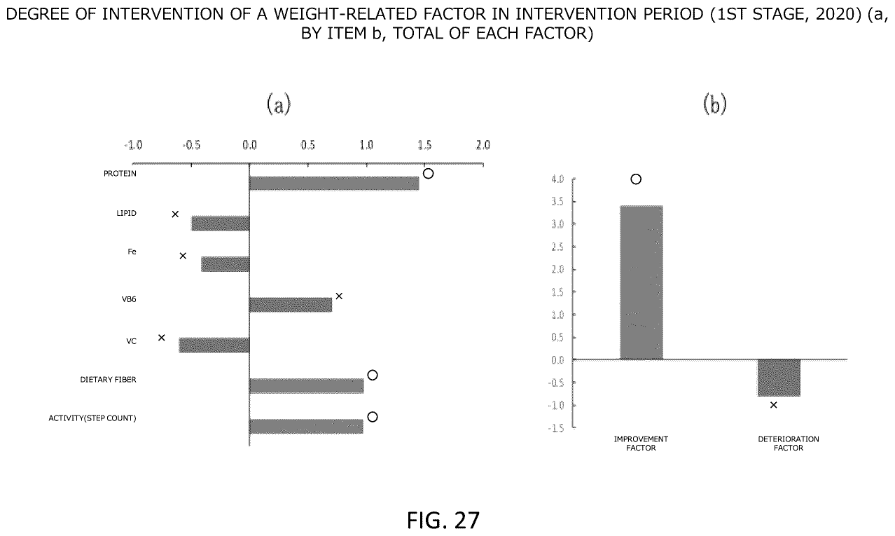

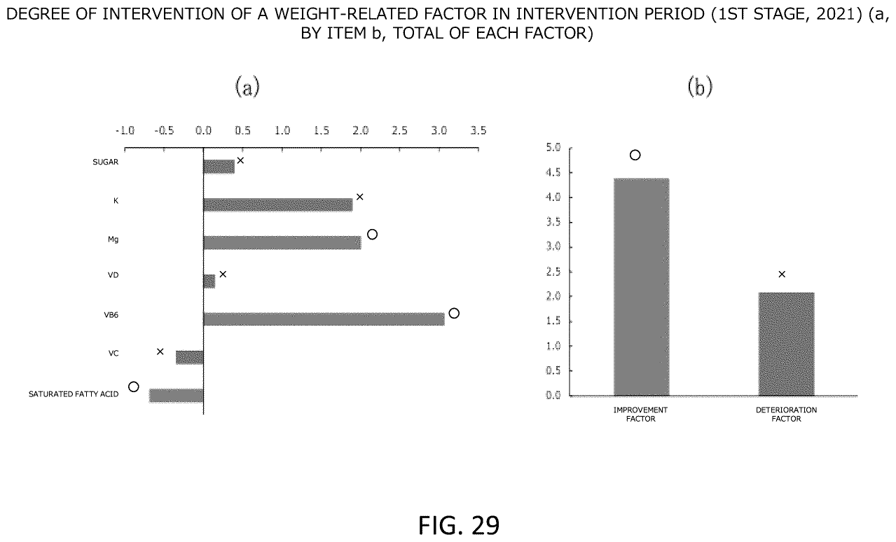

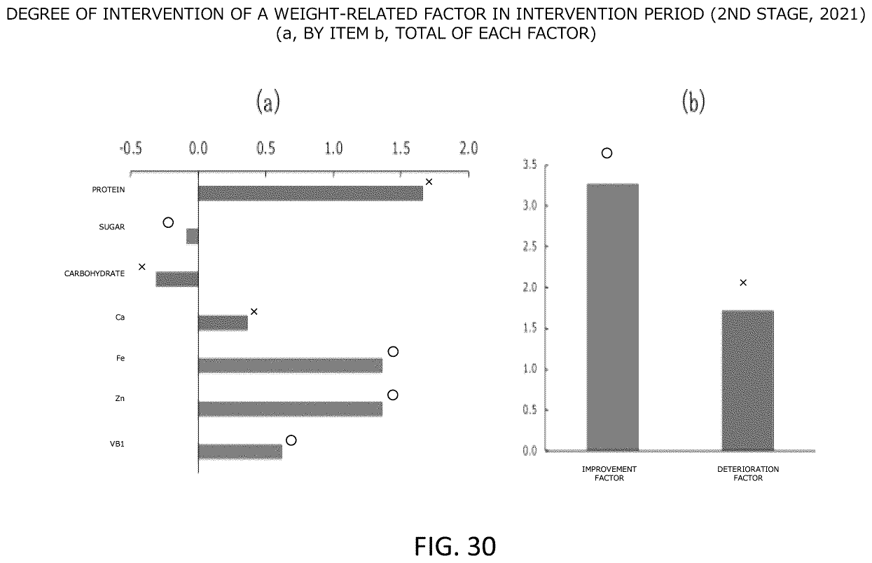

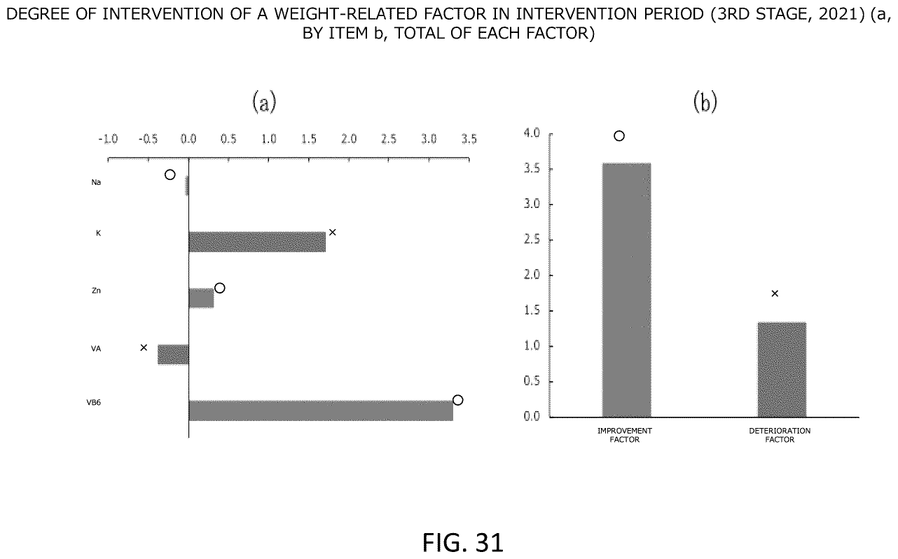

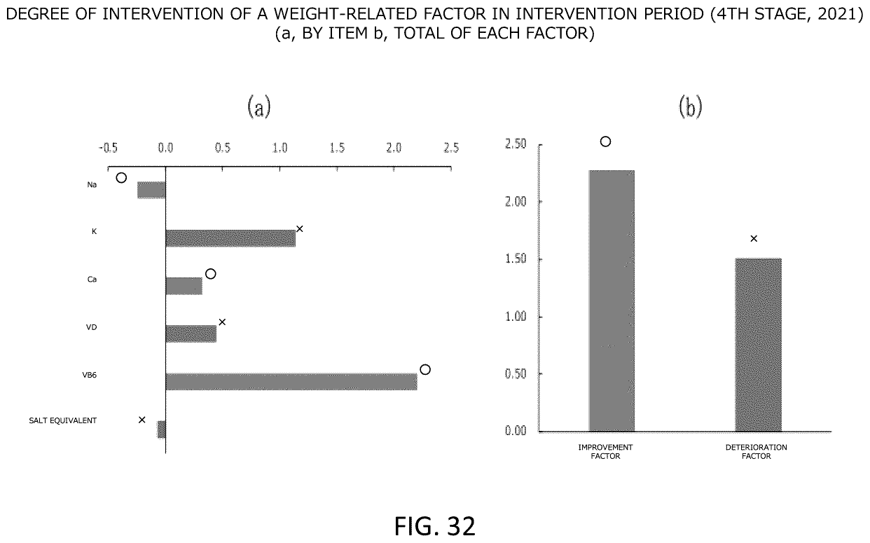

The degree of intervention of the weight-related factor for the test target is shown below (FIGS. 27 to 32). In each drawing, a circle mark indicates the degree of intervention of the body weight improvement factor or the nutrient that is a constituent factor thereof, and a cross mark indicates the degree of intervention of the body weight deterioration factor or the nutrient that is a constituent factor thereof. The degree of intervention having a positive value indicates that the intake of the nutrient increased to be larger than that in the observation period, in the intervention period, and the degree of intervention having a negative value indicates that the intake decreased to be smaller than the intake in the observation period. When the degree of intervention of the body weight improvement factor is a positive value and the degree of intervention of the body weight deterioration factor is a negative value, it indicates that ideal intervention expected to improve the weight of the test subject is being performed.

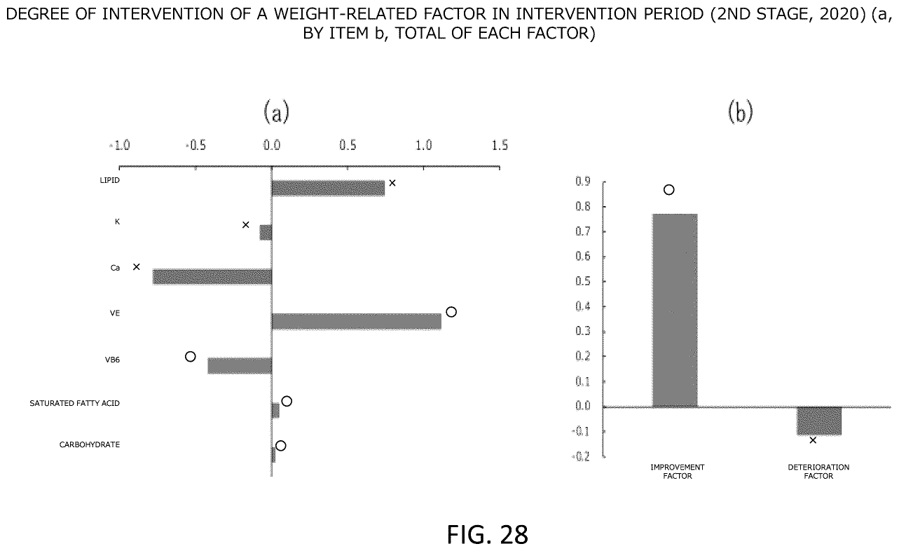

In the feasibility test conducted in 2020, when attention was paid to each weight-related factor, there were nutrients for which ideal intervention could not be performed, such as vitamin B₆ in the first stage of the intervention whose degree of intervention as the deterioration factor became a positive value (FIG. 27a) and vitamin B₆ in the second stage of the intervention whose degree of intervention as the improvement factor became a negative value (FIG. 28a). However, when viewed as the whole related factors, in both cases, the degree of intervention of the improvement factor was a positive value, and the degree of intervention of the deterioration factor was a negative value (the degree intervention of the improvement factor >0, and the degree of intervention of the deterioration factor <0). In addition, in the feasibility test conducted in 2021, for the whole weight-related factors, the degree of intervention of the deterioration factor was a positive value in any of the first to fourth stages of intervention, but the degree of intervention of the improvement factor was increased more than that to cope with the deterioration factor (the degree of intervention of the improvement factor >the degree of intervention of the deterioration factor >0).

As described above, even in a case where the degree of intervention of each weight-related factor is opposite to the ideal, the degree of intervention of the body weight improvement factor is set to a positive value and the degree of intervention of the body weight deterioration factor is set to a negative value in the whole related factors, thereby designing a meal expected to improve the body weight of the test subject. In addition, even if the degree of intervention of the body weight deterioration factor is a positive value, the body weight improvement effect can be similarly expected by increasing the degree of intervention of the body weight improvement factor to the value or the degree of intervention of the body weight deterioration factor or more.

### Blood Test Result

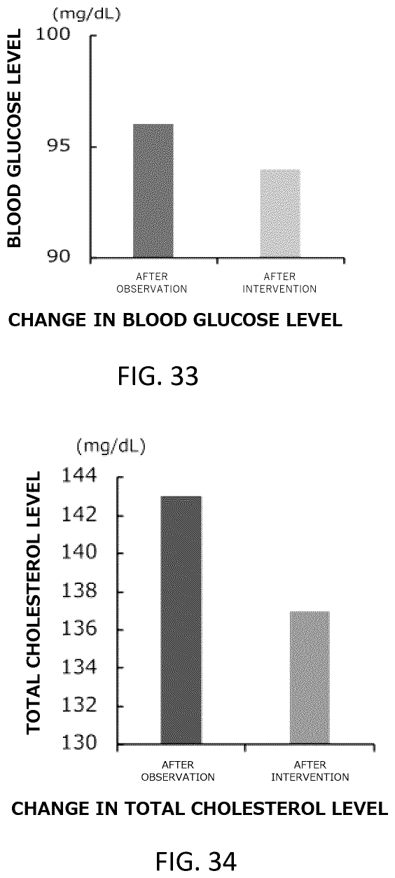

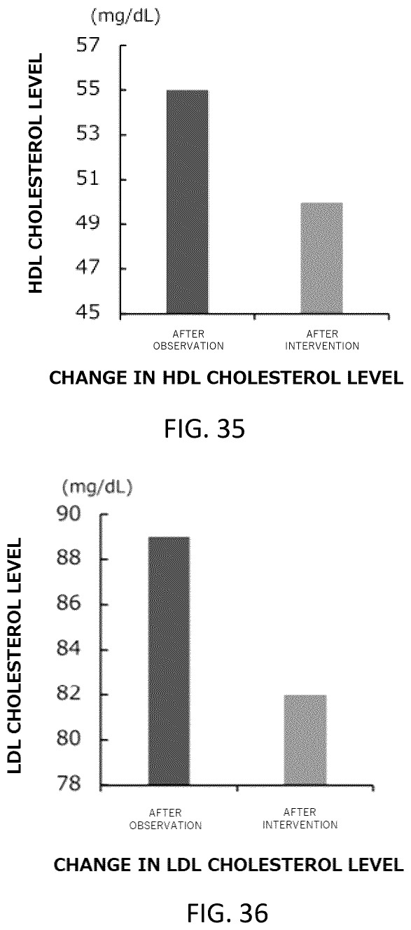

The results of the blood test performed in the feasibility test conducted in 2021 are shown below. As a result of comparing blood components after the end of the intervention period with the observation period, it was confirmed that a blood glucose level and a total cholesterol level decreased (FIGS. 33 to 36).

### Clinical Study Result

### Analysis Target

The amount of the meal ingested by the test subject during the intervention period was adjusted so as to be unchanged from the amount of energy ingested during the observation period, but among nine test subjects who completed the clinical test, test subjects with leftover food were confirmed even during the intervention period. When the leftover food occurs, a nutrient composition is not as designed by nutrient optimization, and it is difficult to verify the effect under the assumed conditions. Therefore, among 28 days of the intervention period, three test subjects who have left food for eight days (30%) or more at breakfast, lunch, or dinner were excluded from targets for efficacy analysis.

As a result, the number of test subjects who became the test subjects for analysis was four (E-02, E-08, E-09, and N-02), and the number of test subjects in the control group was two (E-04, N-06). In addition, among the analysis targets, three underwent the intervention for the purpose of weight gain and one underwent the intervention for the purpose of weight loss.

### Body Weight Prediction Model Formula

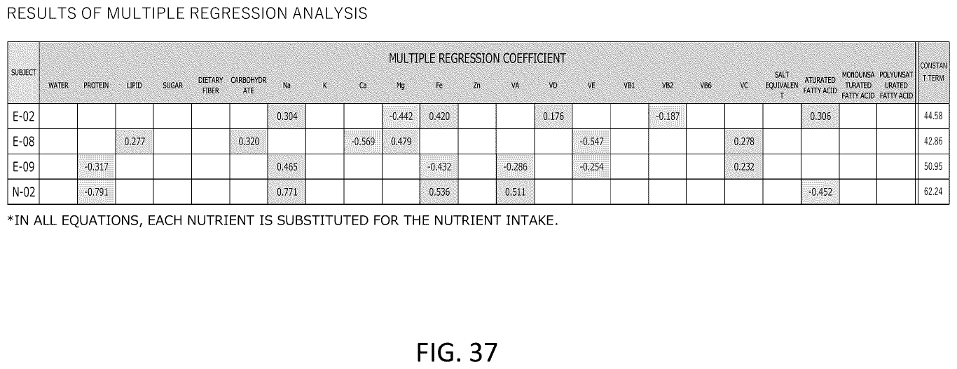

The multiple regression analysis was performed using SPSS Statistics ver. 25 with the wake-up weight acquired in the observation period as an objective variable and the intake nutrient amount a day before the wake-up weight measurement as an explanatory variable, and the results are shown below (FIG. 37).

Based on the obtained multiple regression coefficient, a model formula for predicting the wake-up weight was constructed, and the weight-related factor was specified for each test subject from the sign of the coefficient. An average value of the multiple correlation coefficients of the total of seven model formulas created was 0.64, and a correlation was observed between the intake nutrient and the measured body weight value, suggesting that the wake-up weight can be predicted by the intake nutrient amount. Examples are shown below.

$$E - 02$$

$$Wake - up weight = 0.00013 \times sodium - 0.0051 \times magnesium + 0.1 \times iron + 0.0072 \times vitamin D - 0.32 \times vitamin {B}_{2} + 0.0 \; 21 \times saturated fatty acid + 44.58 \qquad ( Formula 9 )$$

$$E - 08$$

$$Wake - up weight = 0.014 \times lipid + 0.006 \times carbohydrate - 0.0025 \times calcium + 0.0091 \times magnesium - 0.16 \times vitamin E + 0.0038 \times vitamin C + 42.86 \qquad ( Formula 10 )$$

$$E - 09$$

$$Wake - up weight = - 0.018 \times protein + 0.00025 \times sodium - 0.16 \times iron - 0.00084 \times vitamin A - 0.066 \times vitamin E + 0.0027 \times vitamin C + 50.95 \qquad ( Formula 11 )$$

$$N - 02$$

$$Wake - up weight = - 0.032 \times protein + 0.00047 \times sodium + 0.029 \times iron + 0.0017 \times vitamin A - 0.059 \times saturated fatty acid + 62. 24 \qquad ( Formula 12 )$$

### Weight-Related Nutrient Amount of Test Food

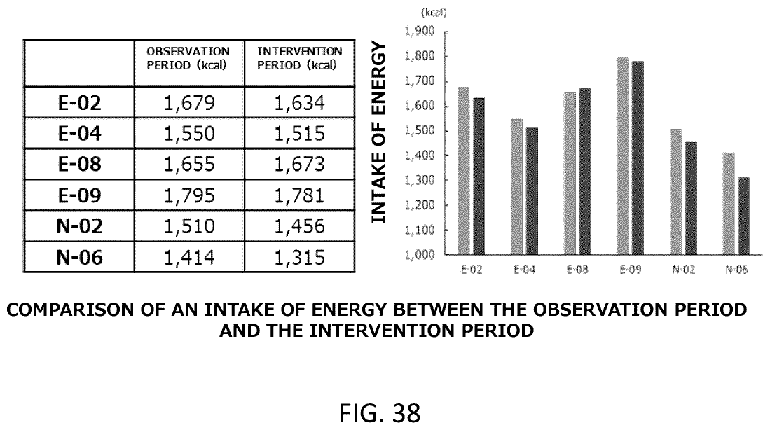

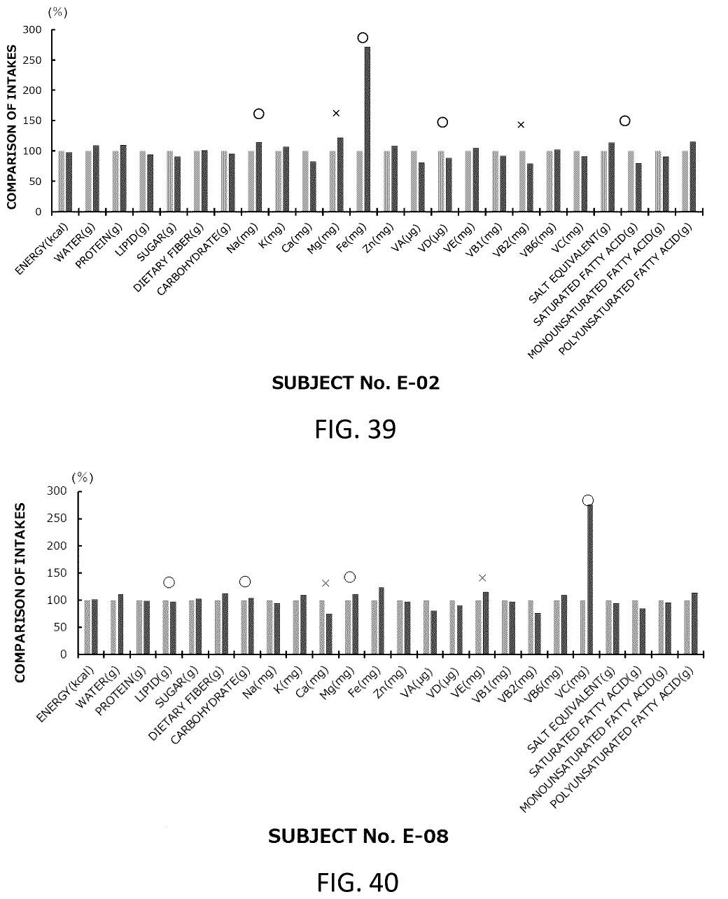

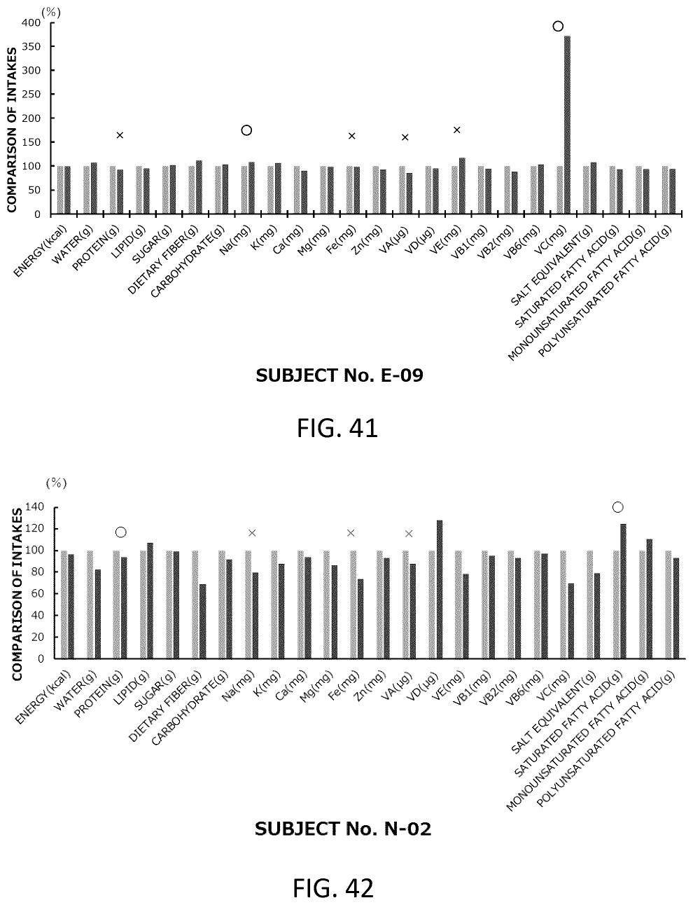

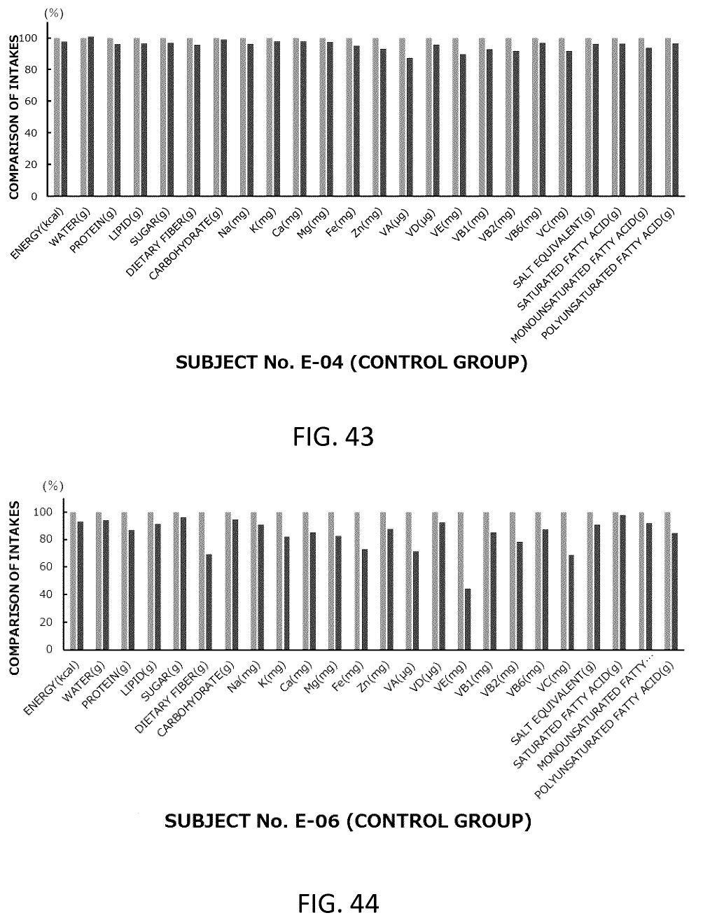

For the seven test subjects allocated to the test group, adjustment of the amount of energy and the amount of each nutrient in the test food ingested during the intervention period were shown in percentage as compared with that ingested during the observation period (FIG. 38). The amount of energy ingested by the test subject was 96.7% on average with respect to that in the observation period, and it was possible to increase/decrease the weight-related factor even under the condition that the amount of energy is not changed. For two test subjects allocated to the control group, only the amount of energy was compared between the observation period and the intervention period (FIGS. 39 to 43). Although dietary intake nutrient optimization was not made, there was no change in amount of energy ingested from the observation period.

The left bar graph indicates the intake during the observation period, and the right bar graph indicates the intake during the intervention period. In each series, a circle mark indicates the body weight improvement factor, and a cross mark indicates the body weight deterioration factor.

### Body Weight Measurement Result

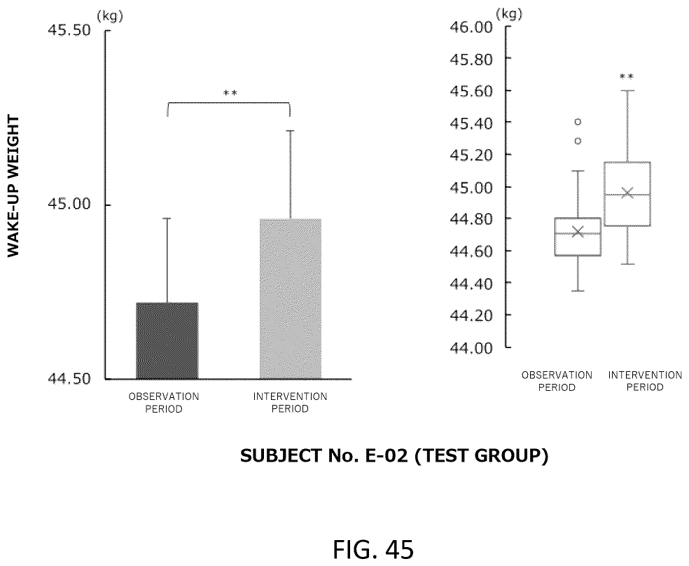

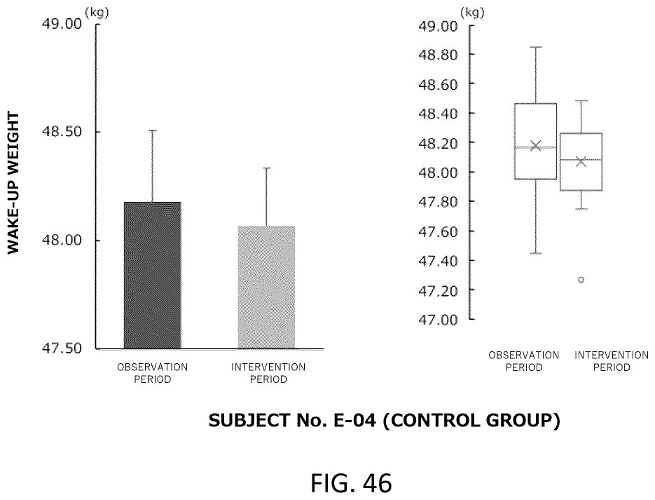

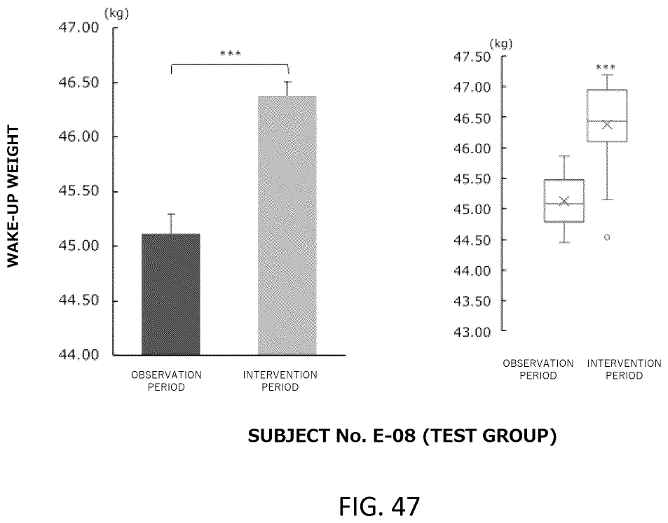

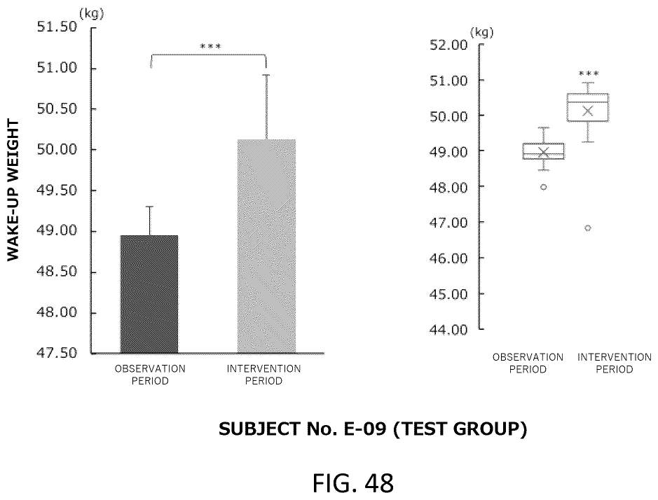

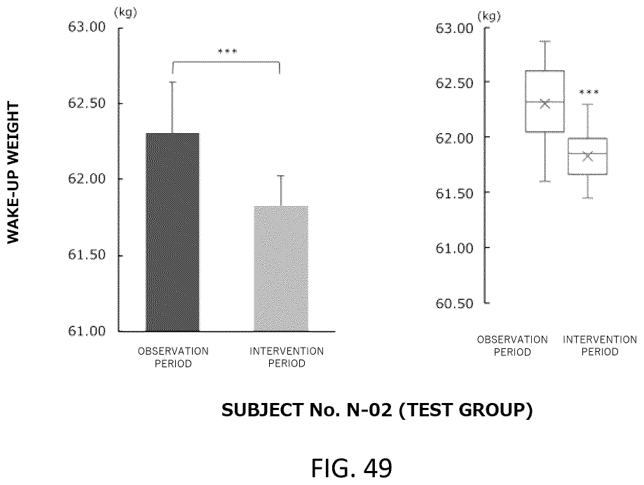

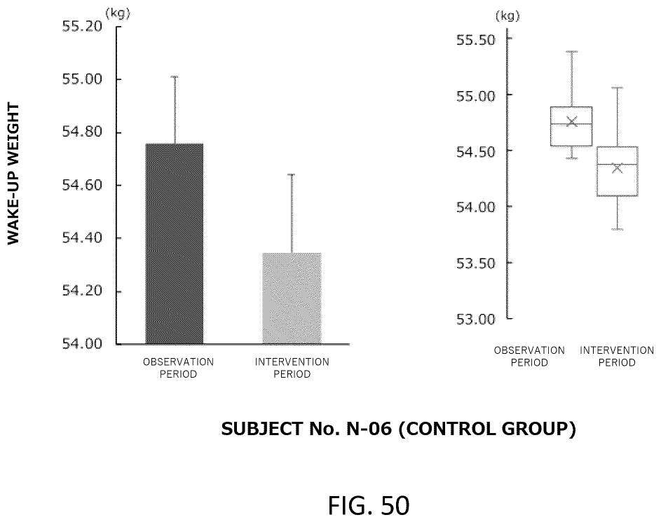

Regarding the wake-up weight of each test subject measured by the body weight and composition monitor, results of comparing the measured values of the observation period and the intervention period are illustrated in FIGS. 45 to 50 below.

### Body Composition Measurement Result

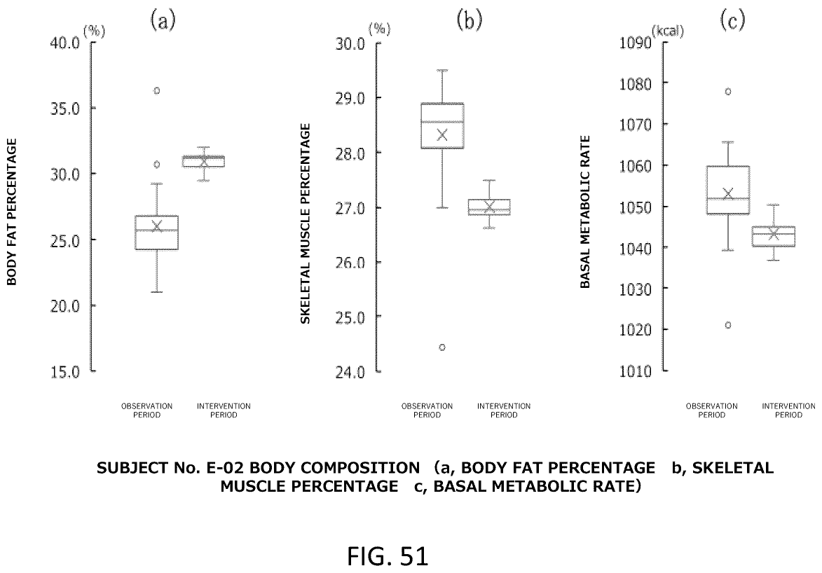

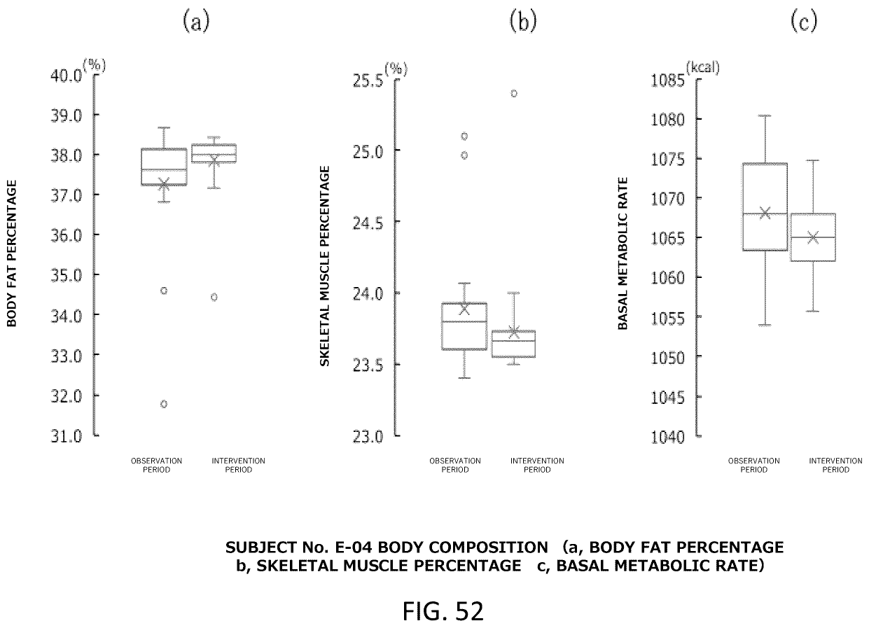

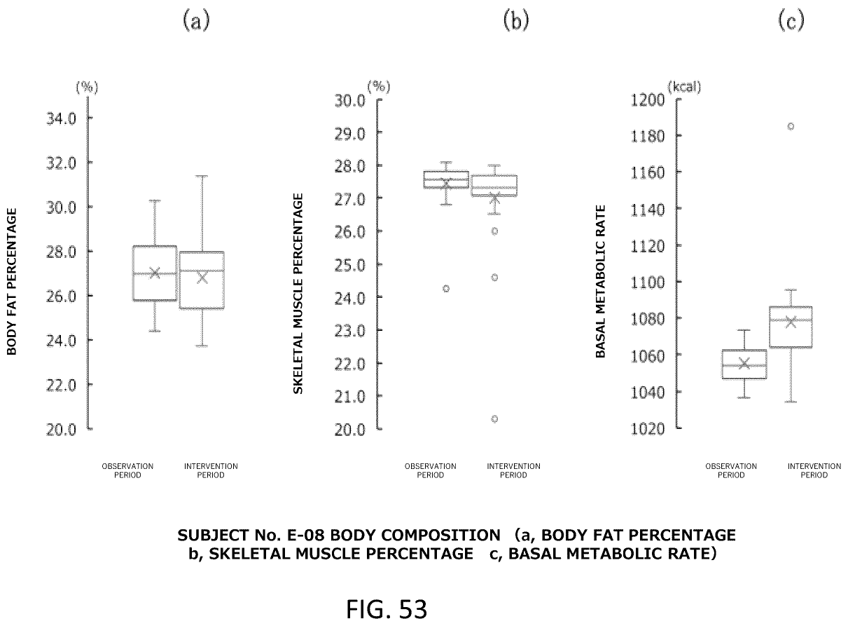

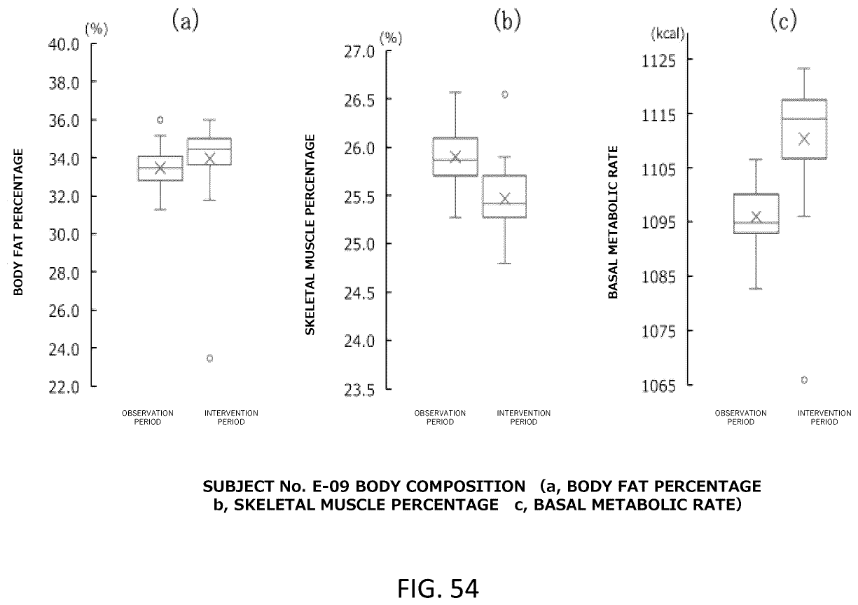

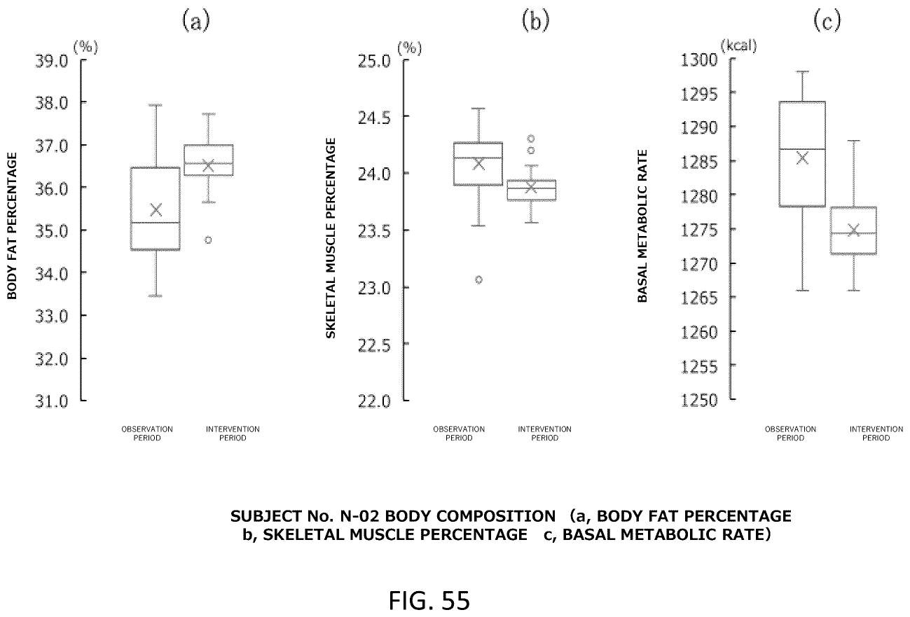

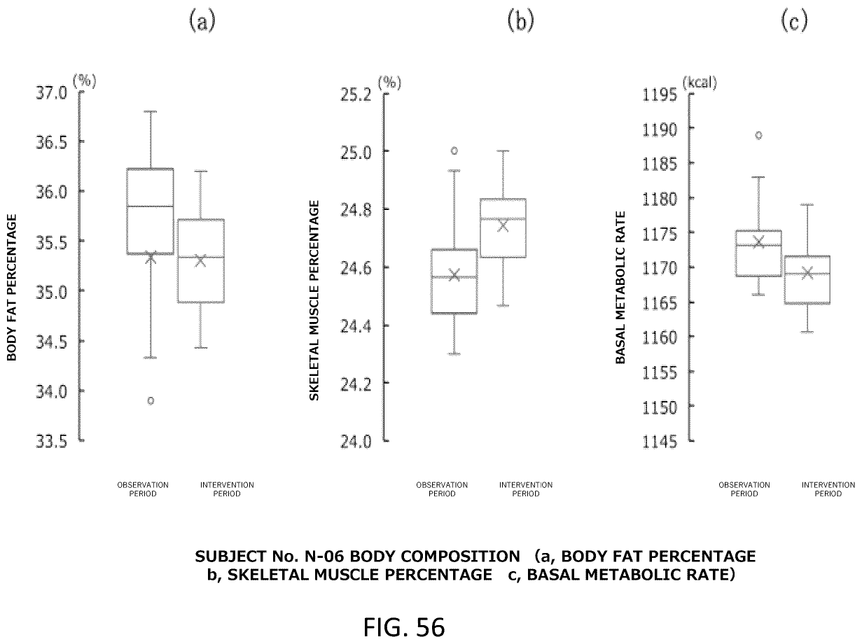

The body fat percentage, the skeletal muscle percentage, and the basal metabolic rate measured by the body weight and composition monitor are illustrated in FIGS. 51 and 56 below. Since the analysis result indicating that the weight-related nutrient varies depending on the test subject was obtained and individual nutrient optimization was performed, it can be confirmed that even persons who similarly underwent adjustment for weight gain exhibit different behaviors such as an increase in body fat percentage, an increase in skeletal muscle percentage, or no change in any of them.

### Evaluation of Degree of Intervention of Nutrient

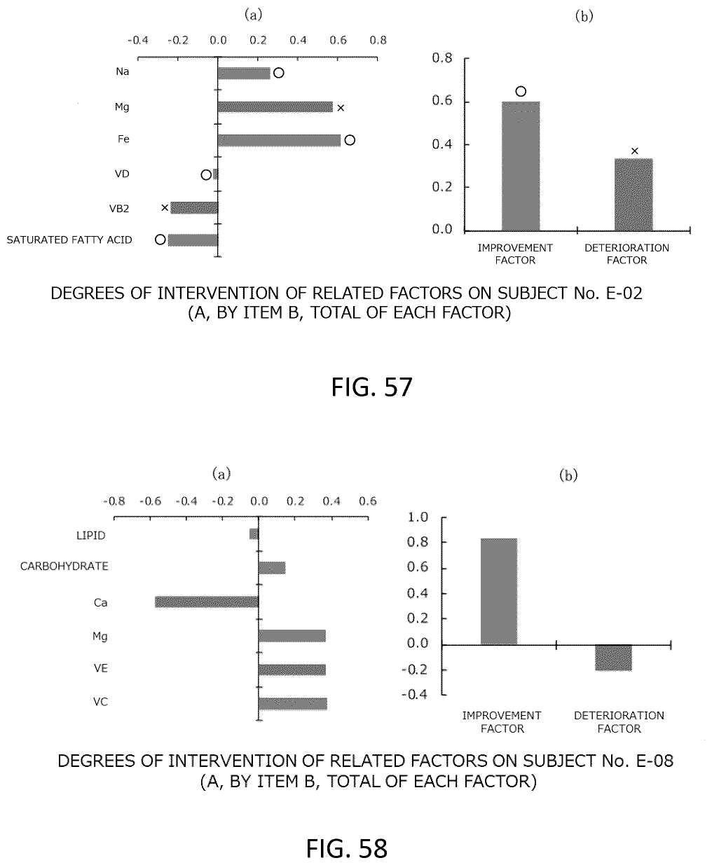

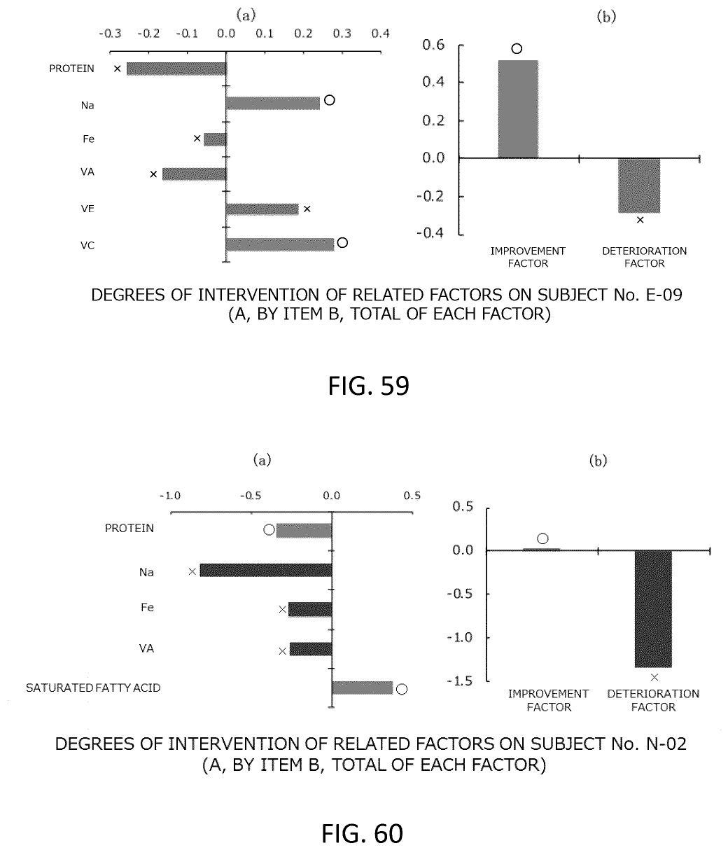

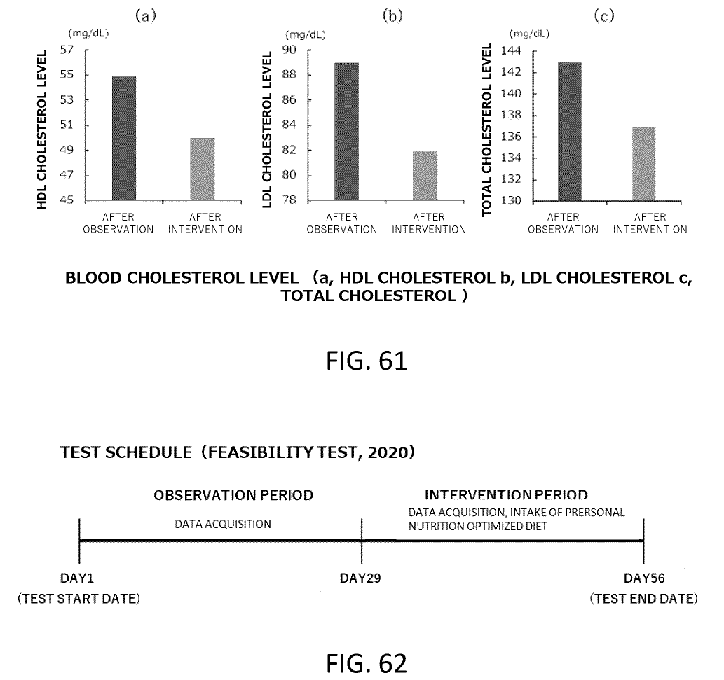

The degree of intervention of the weight-related factor in the intervention period for four analysis targets are illustrated in FIGS. 57 to 60. As in a case shown in Chapter 3, stage 1, 6, there are individual weight-related factors that are improvement factors but have the degree of intervention having a negative value, and individual weight-related factors that are deterioration factors but have the degree of intervention having a positive value (FIGS. 57a to 60a). On the other hand, when viewed in terms of all related factors, in the case of a test subject number E-02, it is shown that the intervention was made such that the degree of intervention of the improvement factor >the degree of intervention of the body weight deterioration factor >0 as in the feasibility test conducted in 2021 (FIG. 57b), in the case of test subject numbers E-08, E-09, and N-02, it is shown that the intervention was made such that the improvement factor >0 and the degree of intervention of the deterioration factor <0, and both of which indicate that the intervention is expected to improve the body weight of the test subject.

### SUMMARY

In the first example, the feasibility test of the nutrient optimization method for effective weight improvement and the clinical test for demonstrating the body weight improvement effect by personal nutrient optimization were conducted.

As a result of analyzing the relationship between the body weight and the intake nutrient amount of an individual, it was shown that the nutrients incorporated into the body weight prediction model formula differed depending on the individual, and the weight-related factor differed depending on the individual. In the 28-day intervention feasibility test with the optimized nutrient means, which was conducted three times in total, significant weight improvement (−2.67 kg, −1.85 kg, and −3.01 kg with respect to the weight before the intervention, p<0.001 in any case) was confirmed in any intervention. In the continuous analysis performed every week, a different model formula was obtained every time the analysis was performed, and magnesium specified as the body weight improvement factor was excluded from the related factor in the next analysis, and sugar that was the body weight deterioration factor was specified as the body weight improvement factor in the next analysis. Therefore, it was suggested that even for the same individual, nutrients serving as the weight-related factors changed depending on changes in the environment in the body and an external environment. In the intervention using the continuous analysis, −3.01 kg (weight loss of 4.2% of the total body weight) was achieved with respect to the weight before the intervention, and the greatest body weight improvement effect was obtained among the feasibility tests conducted so far. From this, it is considered that the continuous analysis is useful for continuously obtaining the body weight improvement effect.

In the results of the clinical test, among seven test subjects allocated to the test group, three test subjects who had left food in eight meals (30%) or more of 28 meals of all the intervention meals did not satisfy a reference intake nutrient amount, and thus were excluded from the analysis target. As for four test subjects who are targets for efficacy analysis, significant weight improvement (p<0.01) was observed in four test subjects (100%) before and after the intervention. Since no significant body weight change was observed in the control group, it was estimated that any body weight change achieved by the test was due to optimized nutrient adjustment.

These results demonstrate that it is possible to predict the body weight from the intake nutrient amount, and it is possible to bring the body weight close to a healthy body weight by adjusting the amount of nutrients that are the improvement factor and the deterioration factor specified by the multiple regression analysis leading to the relationship between the body weight and the nutrients, that is, by continuously ingesting a meal with optimized nutrients for an individual. In the study aimed at body weight improvement, it was defined that the intake energy amount was not changed as a condition for performing nutrient optimization. It can be said that the test results, which have demonstrated the body weight improvement effect without changing the amount of energy, that is, without relying on caloric control, overturn conventional theories on weight loss.

In the analysis for specifying the weight-related factors that affect the weight of an individual, the analysis method used in the present example has a feature that it is possible to incorporate not only the nutrient amount but also any numerical value. In fact, in the feasibility test, the step count as the amount of exercise was incorporated as a factor other than nutrients at the time of analysis, and as a result, the step count was specified as the body weight improvement factor in the first stage of the intervention of the feasibility test conducted in 2020. In the first stage of the intervention, in addition to the ingestion of the meal with optimized nutrients, the test target was allowed to increase his or her walking within a reasonable range. An expected value of a body weight improvement amount expected by walking was considered to be 0.4 kg as a result of calculation based on the amount of energy required for burning of 1 kg of fat (kcal) and the amount of energy consumed by walking (kcal) with reference to the amount of energy consumed during walking presented by the American Society of Sports Medicine. This corresponds to about 15% of 2.7 kg of body weight improvement actually observed. From this, it was inferred that increasing the amount of exercise has an effect of assisting the weight improvement effect of nutrient optimization, but the effect is smaller than that of the amount of nutrients. The amount of exercise (step count) was specified as the weight-related factor only at this time, and was not specified as the weight-related factor in the second stage of the intervention or the test conducted in 2021.

In the test conducted in the first example, the “amount of various nutrients included in the meal” is an important numerical value necessary for specifying nutrients relating to the body weight. In estimating the numerical value, a smartphone application “asken” was used in the feasibility test, and WEB software “Mr. KenDateman web” was used in the clinical test. Both systems are a system in which a nutrient amount is provided with reference to the Standard Tables of Food Composition in Japan when a meal menu name or an ingredient name is input, and prediction accuracy of “asken” including commercially available food in addition to the composition table is also reported in literatures. The Standard Tables of Food Composition in Japan is obtained by analyzing the amount of various minerals and vitamins in addition to three major nutrients by designated analysis methods, and calculating and recording the analysis results as estimated values, but these pieces of data are not absolute values. This is because the amount of nutrients is not uniquely determined because there are many differences such as seasons, production areas, and varieties depending on ingredients. Nevertheless, in this study, it was considered that the determination of the related factor can be made from an approximate value of the amount of nutrients included in the ingredient.

For the feasibility test conducted in 2021, after the start of the intervention period, linear approximation with y as the body weight (kg) and x as the number of days (days) from the start of use of the model formula was derived for one-week weight change every time the model formula was updated by reanalysis, and the slopes were calculated. As a result, the slopes of four periods were −0.27, −0.15, −0.03, and −0.20, respectively, and it was confirmed that the body weight was continuously improved. Estimating from the slopes, the body weight improvement of 1 kg is expected in about one week. Although the analysis was performed every week in the test conducted in this study, if a time required for nutrient optimization adjustment of the meal based on the body weight prediction model formula updated by the analysis can be shortened, it is theoretically possible to perform the analysis once a day and then update the model formula. This continuous analysis is considered to be a useful method for quickly reflecting a status of the body that changes from moment to moment due to an external factor such as an environment around the test subject and specifying nutrients that affect a health index of an individual with higher accuracy. In the continuous analysis, the same nutrient is not necessarily selected in the model formula, and thus it is considered that the update of the model formula is expected to enrich the variation of a meal pattern, and it is possible to secure the quality of light (QOL). It was inferred that the update of the model formula by the continuous analysis copes with a temporal change of the body composition and is also useful from the viewpoint of securing the QOL of the test subject. From the above results, it was determined that the test conducted in the first example showed that prediction and control of the body weight in addition to the blood pressure can also be performed by the ingested nutrients of the meal.

The degree of adjustment of nutrients which are weight-related factors was evaluated by newly setting and calculating an index called the degree of intervention in the first example. Although it is ideal to perform adjustment to increase the amount of nutrients that are the body weight improvement factors and to decrease the amount of nutrients that are the body weight deterioration factors, it is assumed that the above-described ideal adjustment cannot be performed in order to ensure the strong desire of the test subject and the QOL. Specifically, for a test subject who has a desire to “increase a protein intake” despite protein being the deterioration factor, it is necessary to reduce other deterioration factors by the amount of increase in protein to reduce the total degree of intervention of the deterioration factors so that the goal can be achieved. In this case, “protein that is the individual deterioration factor increases”, but “the total degree of intervention of the deterioration factor decreases”. In the reverse pattern, the “individual improvement factor decreases”, and “the total degree of intervention of the improvement factor increases” in some cases. In addition, even in a case where the individual improvement factor decreases, “the total degree of intervention of the improvement factor decreases”, and the individual deterioration factor increases, if adjustment is made such that “a decrease in total sum of the deterioration factors exceeds an effect of a decrease in total sum of the improvement factors”, the goal can be achieved. Conversely, even in a case where the individual deterioration factor increases, “the total degree of intervention of the deterioration factor increases”, and the individual improvement factor decreases, if “the total degree of intervention of the improvement factor exceeds the total degree of intervention of the deterioration factor (the degree of intervention of the improvement factor >the degree of intervention of the deterioration factor >0)”, the goal is expected to be achieved. From this, it was inferred that the degree of freedom is extremely high in the optimization of nutrients for achieving the goal of an individual.

As described above, in the first example, the body weight improvement effect by the personal optimization of the intake nutrient was demonstrated by the clinical test for humans. In addition to body data such as the skeletal muscle percentage and the brain function in addition to the body weight, numerical value data such as a sports competition time is also used as an improvement index to perform a test of optimizing the intake nutrient and measuring the effect, and a certain result is obtained in each of the tests. These results are considered to indicate that the improvement effect can be obtained by personal optimization of nutrients if quantifiable in relation to a biomarker, and health desired by an individual can be achieved, that is, self-realization can be achieved.

### Second Example

In a second example, the brain function (memory) is the improvement target. A correlation between a daily intake nutrient amount and a memory test was analyzed, and a memory improvement effect was verified by designing and ingesting an optimal personal diet optimized for the nutrient amount.

### Experimental Material and Method

### Experimental Material

Test Subject
One healthy adult male

During the test period, three meals were taken per day, ingestion of health foods, supplements, and alcohols and ingestion of meals other than three meals (additional meals and snacks) were prohibited, and beverages were limited to water only. In addition, an irregular life was avoided, and a consistent exercise habit was maintained.

### Statistical Software Application

IBM SPSS Statistics ver. 25 (IBM, USA)
APPRON (Health Nutrition Support Center Corporation, Osaka)
* Based on the Standard Tables of Food Composition in Japan (eighth edition, 2020)

### Experimental Method

Test Schedule (FIG. 62)

The test period was divided into two periods: an observation period (28 days) and a demonstration period (28 days). In the observation period, a memory test score and the number of times the memory test was performed were recorded, and an intake nutrient amount was also recorded using a nutrient amount calculation software. In the demonstration period, data acquired in the observation period was subjected to multiple regression analysis, and an optimal personal diet obtained by adjustment based on the analysis result was taken as three meals (breakfast, lunch, and dinner), and the memory test score, the number of times, and the intake nutrient amount were recorded as in the observation period.

### Memory Test

In the second example, as an index of memory, a test for measuring short-term memory using numbers is used. The memory test was conducted six to ten times a day, and the number of tests was randomly distributed. Six of the tests were conducted at designated times (twice each at 10:00, 13:00, and 22:00), and the remaining 0 to 4 tests were conducted at arbitrary times. In order to perform comparison before and after the test under the condition that there is no difference in the test time and the number of tests, an average of the test scores of the six tests conducted at the designated times was taken as a score of the day. In order to eliminate an increase in score due to habituation, the same memory test was conducted every day before the start of the main test, and the main test was started after an increase in score was no longer confirmed.

### Memory Test Procedure

1. The test subject memorizes numbers displayed in random arrangement in a 5×7 grid.
2. The numbers are hidden after 3 seconds.
3. The test subject sequentially selects numbers from 1 so as to reproduce the displayed numbers.
4. Since the number increases each time the test subject selects a correct number, the largest displayed number until the test subject selects a wrong number becomes the score.

### Derivation of Model Formula

In designing a memory-improving diet, it is necessary to analyze a relationship between the memory and nutrients, and specify nutrients that are improvement factors and deterioration factors for the memory. Multiple regression analysis using SPSS was performed to specify memory-related nutrients and determine their impact on the formula to derive the model formula. A backward elimination method was used as the analysis method with the memory test score as an objective variable and the number of memory tests and an intake of a total of 20 types of nutrients ingested a day before the test (protein, lipid, a total dietary fiber content, carbohydrate, sodium, potassium, calcium, magnesium, iron, zinc, vitamin A, vitamin D, vitamin E, vitamin B₁, vitamin B₂, vitamin B₆, vitamin C, salt equivalent, saturated fatty acid, and sugar) and energy as explanatory variables. In order to reflect the latest data in the model formula, a total of seven analyses were performed every four days using data for the most recent 24 days from an analysis time point. In addition, in a case where there is a high correlation between the explanatory variables, reliability of the regression formula decreases, and a prediction error may increase. Therefore, a model formula using an explanatory variable having a variance inflation factor (VIF) of 10 or less was adopted. In a case where the VIF was 10 or more, measures such as shifting data to be analyzed by one day and increasing the number of pieces of data were taken. In the model formula, a selected nutrient is a variable selected by the multiple regression analysis to predict the memory, and a nutrient having a positive multiple regression coefficient is considered to be a factor having an influence such as a memory improvement factor, and a nutrient having a negative multiple regression coefficient is considered to be a factor having an influence such as a memory deterioration factor.

### Design of Optimal Personal Diet

The optimal personal diet capable of achieving a prediction point was designed by setting a target prediction point of the memory test and adjusting the memory improvement factor and the memory deterioration factor clarified by the derived model formula. The total daily intake nutrient amount was substituted into the model formula, and the meal was designed and optimized by performing operations such as changing an ingredient to be used and adjusting an ingredient use amount such that the calculated prediction point becomes a set target point. In order to maintain the QOL, three meals (breakfast, lunch, and dinner) were taken per day, and designed to be within ±200 kcal (±11.6%) as compared with an average energy intake during the observation period so as not to largely deviate from the meals taken during the observation period.

### Nutrient Adjustment Degree Evaluation

The intake nutrients were standardized, and the degree of adjustment of each nutrient was calculated and evaluated. The standardization is to perform scaling such that an average of data becomes 0 and a variance becomes 1, and the standardization enables comparison of nutrients with different units. The average and standard deviation of nutrient data used in the analysis were used for the standardization, and a value obtained by multiplying a value obtained subtracting the nutrient amount after the standardization in the observation period from the average of the nutrient amount after the standardization in the demonstration period was multiplied by an absolute value of a standardization coefficient derived by the multiple regression analysis was used as the degree of adjustment. The degree of adjustment was calculated for the overall degree of adjustment of the memory-related factors and the individual degree of adjustment of the memory-related factors. When the value of the degree of adjustment is positive, the adjustment is performed more than the observation period, and when the value is negative, the adjustment is performed less than the observation period.

### Significant Difference Test

As a result of the memory test, an average value and a standard deviation were calculated for each observation period and each demonstration period. Since a result corresponding to a certain improved food was a result of the next day, memory test data on the first day of the demonstration period was excluded. For comparison of the memory test result between the observation period and the intervention period, statistical processing was performed by an unpaired t-test. A significance level in the test was 5% or less in a two-sided test (*p<0.05, **p<0.01, and ***p<0.001).

### Experimental Result

### Specification of Memory-Related Factor

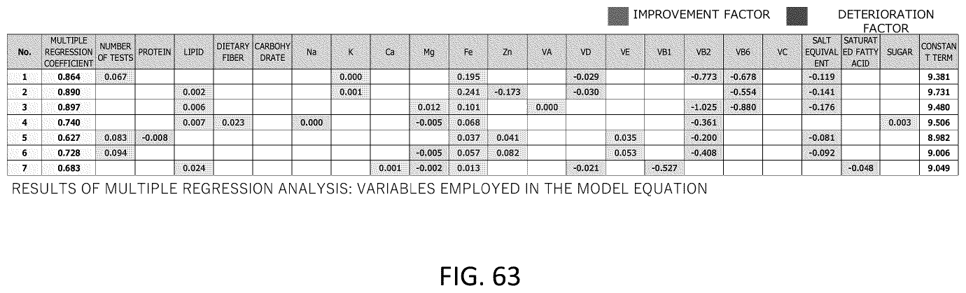

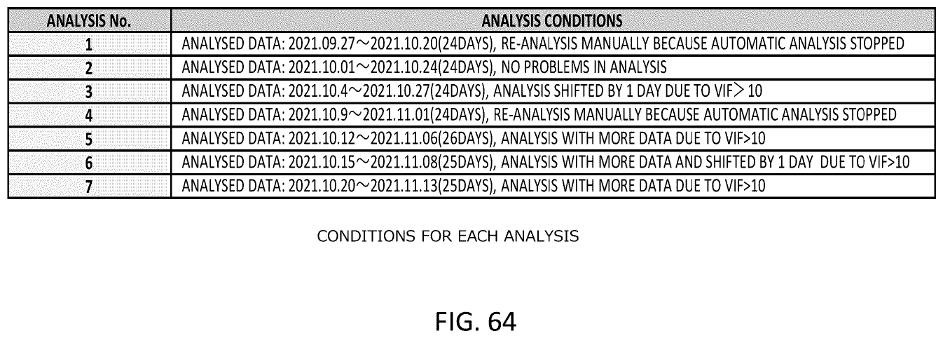

The multiple regression analysis was performed using data of the intake nutrient amount and the number of memory tests of the previous day acquired in the observation period as the explanatory variables and the memory test score as the objective variable. The model formula for predicting the memory test score was derived from the obtained multiple regression coefficient, and the improvement factor and the deterioration factor were specified from the sign of the coefficient (FIG. 63). An average correlation coefficient of seven analyses was 0.776, and a high correlation was observed. In addition, there were different types of improvement factors and deterioration factors in each analysis. Conditions for each analysis are illustrated in FIG. 64.

### Memory Test Score Prediction Model Formula

$$Correlation coefficient : 0.084$$

$$Memory test score = 0.0667 [ number of {tests}_{times} ] + 0.00049 [ {potassium}_{m g} ] + 0.1 \; 95 [ {iron}_{m g} ] - 0.0289 [ vitamin {D}_{μ g} ] - 0.773 [ vitamin {B}_{2 m g} ] - 0.678 [ vitamin {B}_{6 m g} ] - 0.119 [ salt {equivalent}_{g} ] + 9.381 \qquad Analysis No . 1$$

$$Correlation coefficient : 0.89$$

$$Memory test score = 0.00207 [ {lipid}_{g} ] + 0.00069 [ {potassium}_{m g} ] + 0.241 [ {iron}_{m g} ] - 0.173 [ {zinc}_{m g} ] - 0.0301 [ vitamin {D}_{μ g} ] - 0.554 [ vitamin {B}_{6 m g} ] - 0.141 [ salt {equivalent}_{g} ] + 9.731 \qquad Analysis No . 2$$

$$Correlation coefficient : 0.897$$

$$Memory test score = 0.00627 [ {lipid}_{g} ] + 0.012 [ {magnesium}_{m g} ] + 0.101 [ {iron}_{m g} ] + 0.000087 [ vitamin {A}_{μ g} ] - 1.0247 [ vitamin {B}_{2 m g} ] - 0.88 [ vitamin {B}_{6 m g} ] - 0.176 [ salt {equivalent}_{g} ] + 9 \text{.480} \qquad Analysis No . 3$$

$$Correlation coefficient : 0.74$$

$$Memory test score = 0.00707 [ {lipid}_{g} ] + 0.0232 [ total dietary fiber {content}_{g} ] - 0.000331 [ {sodium}_{m g} ] - 0.00508 [ {magnesium}_{m g} ] + 0.68 [ {iron}_{m g} ] - 0.361 [ vitamin {B}_{2 m g} ] - 0.0031 [ {sugar}_{g} ] + 9.506 \qquad Analysis No . 4$$

$$Correlation coefficient : 0.627$$

$$Memory test score = 0.0831 [ number of {tests}_{times} ] - 0.00821 [ {protein}_{g} ] + 0.0368 [ {iron}_{m g} ] + 0.0407 [ {zinc}_{m g} ] + 0.0347 [ vitamin {E}_{m g} ] - 0.2 [ vitamin {B}_{2 m g} ] - 0.0809 [ salt {equivalent}_{g} ] + 8 \text{.982} \qquad Analysis No . 5$$

$$Correlation coefficient : 0.728$$

$$Memory test score = 0.0944 [ number of {tests}_{times} ] - 0.00498 [ {magnesium}_{m g} ] + 0.0571 [ {iron}_{m g} ] + 0.0822 [ {zinc}_{m g} ] + 0.0526 [ vitamin {E}_{m g} ] - 0.408 [ vitamin {B}_{2 m g} ] - 0.0923 [ salt {equivalent}_{g} ] + 9 \text{.006} \qquad Analysis No . 6$$

$$Correlation coefficient : 0.683$$

$$Memory test score = 0.0239 [ {lipid}_{g} ] + 0.000877 [ {calcium}_{m g} ] - 0.00237 [ {magnesium}_{m g} ] + 0.0133 [ {iron}_{m g} ] - 0.0206 [ vitamin {D}_{μ g} ] - 0.527 [ vitamin {B}_{1 m g} ] - 0.0481 [ saturated fatty {acid}_{g} ] + 9.049 \qquad Analysis No . 7$$

### Design of Optimal Personal Diet

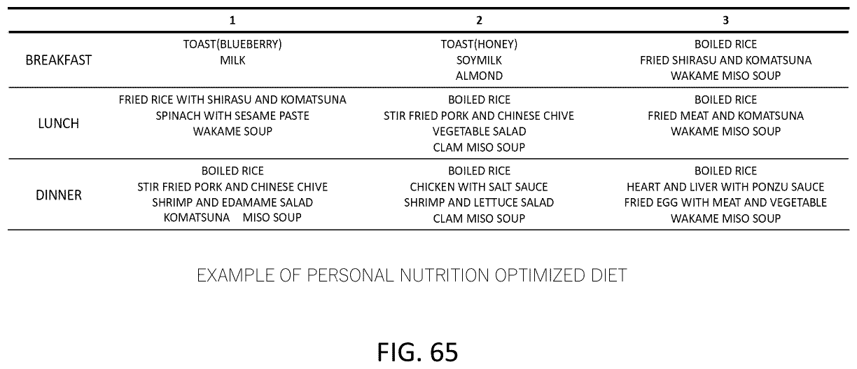

A target prediction point was set based on the memory test score of the observation period, and the optimal personal diet (three meals a day) that can achieve the target prediction point was designed. The prediction point was set to 10.3 point, and a menu was created by adjusting ingredients. An example of the menu is illustrated in FIG. 65.

### Degree of Adjustment of Nutrient

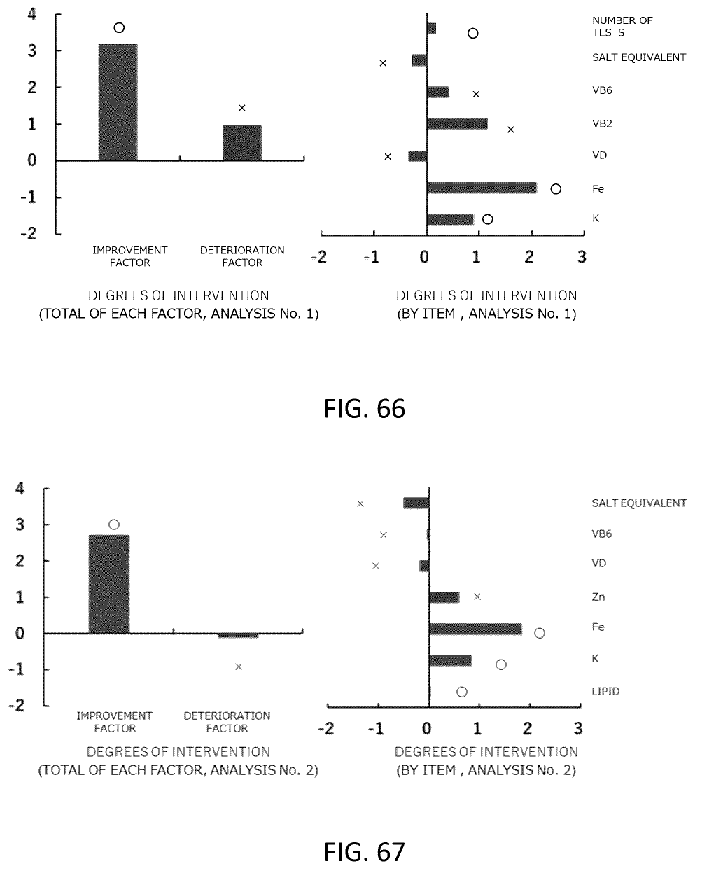

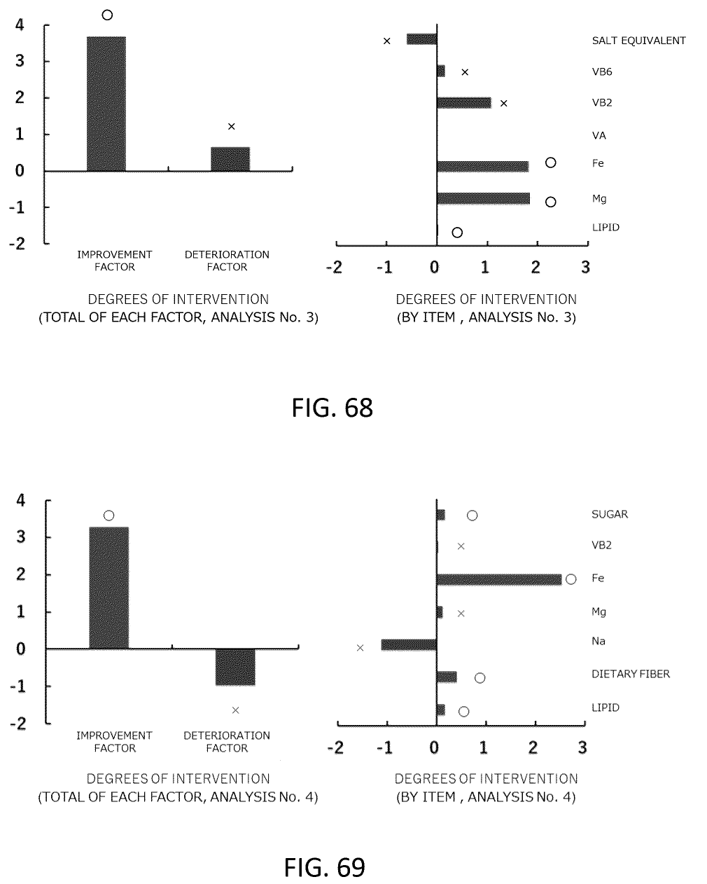

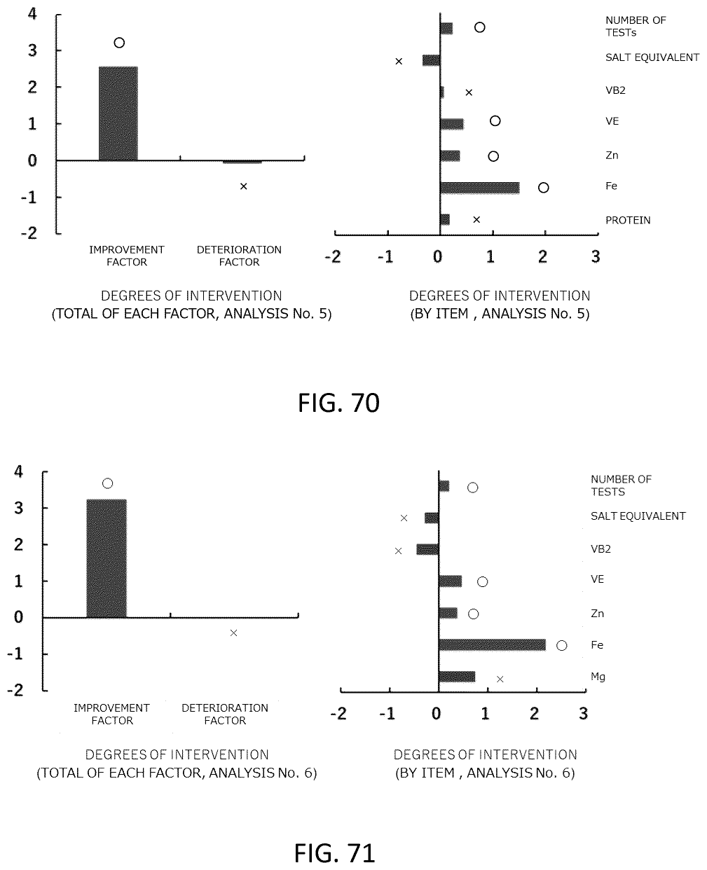

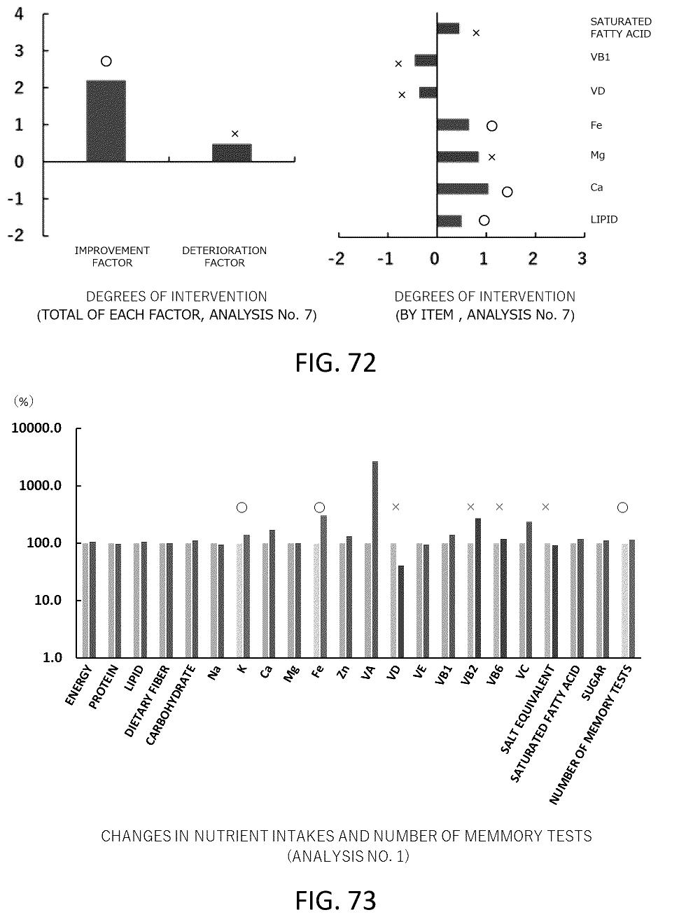

The overall degree of adjustment and the individual degree of adjustment of the memory improvement factors and the memory deterioration factors specified in each analysis are shown below (FIGS. 66 to 72). Although the degrees of adjustment of all the improvement factors are positive values, the degrees of adjustment of the deterioration factors were not uniform, and were positive values in Analysis Nos. 1, 3, 6, and 7 and were negative values in Analysis Nos. 2, 4, and 5. Ideally, individual improvement factors are increased and individual deterioration factors are decreased as nutrient adjustment, but ideal adjustment may not be possible in order to maintain the QOL. As an example, in a case where it is not desired to decrease or increase an intake of protein which is a deterioration factor, it is necessary to perform adjustment such as reducing other deterioration factors and increasing the improvement factor. In addition, even in a case where the individual improvement factor decreases, adjustment can be performed by increasing other improvement factors and decreasing the deterioration factors. In this way, it is possible to design the optimal personal diet that has achieved the target value by performing adjustment such that “an effect by the sum of the improvement factors exceeds an effect by the sum of the deterioration factors”.

### Change in Intake Nutrient Amount

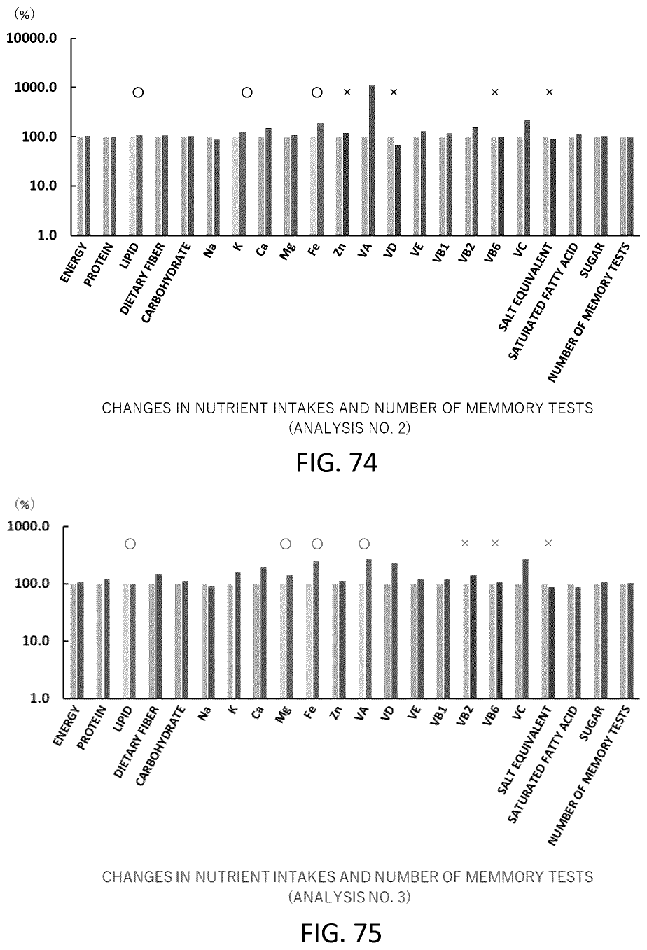

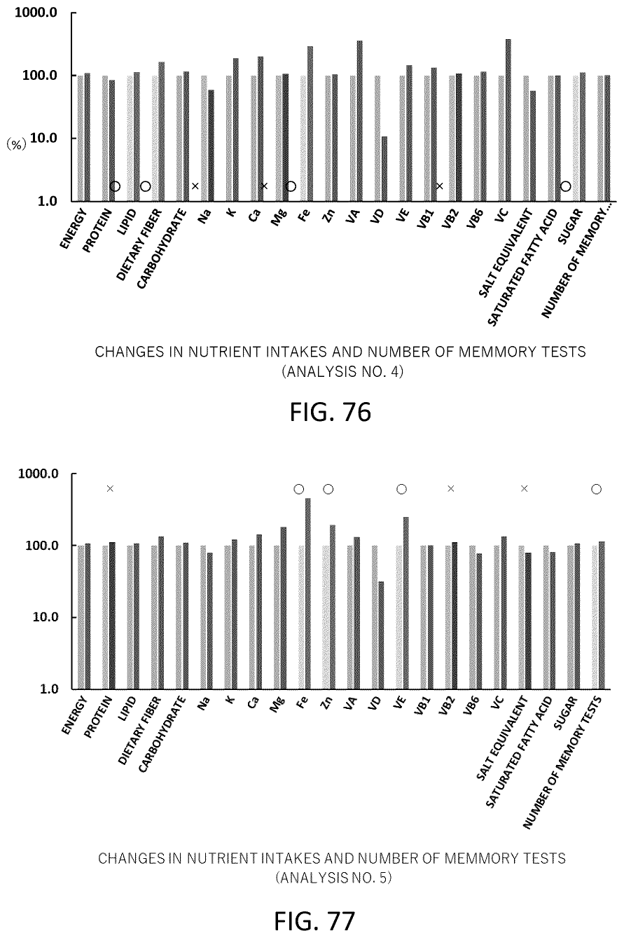

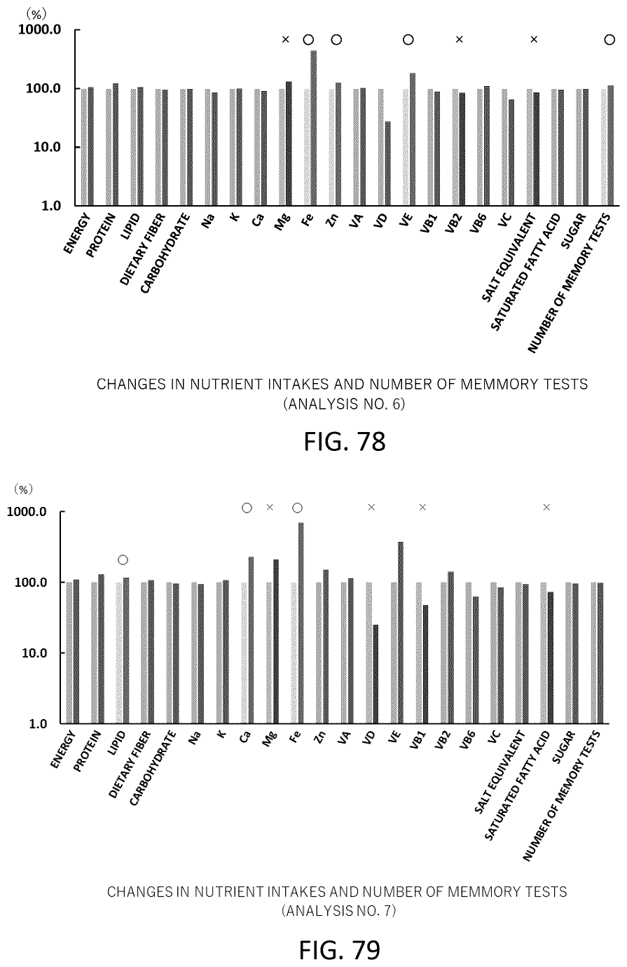

To compare the intake nutrient amount between the observation period and the demonstration period, graphs illustrating comparison with the intake during the observation period as 100% are illustrated (FIGS. 73 to 79). In two bar graphs with the same label, the left bar graph indicates the observation period, the right bar graph indicates the demonstration period, a circle mark indicates the memory improvement factor, and a cross mark indicates the memory deterioration factor. The energy amount was designed to be almost equal to an average intake energy amount during the observation period. The energy amount during the demonstration period was 104 to 109%, and the design satisfying the conditions could be achieved. The number of memory tests was adjusted so as to increase for the improvement factor and to be almost equal to the average number of memory tests of the observation period for factors other than the improvement factor.

### Memory Test Results

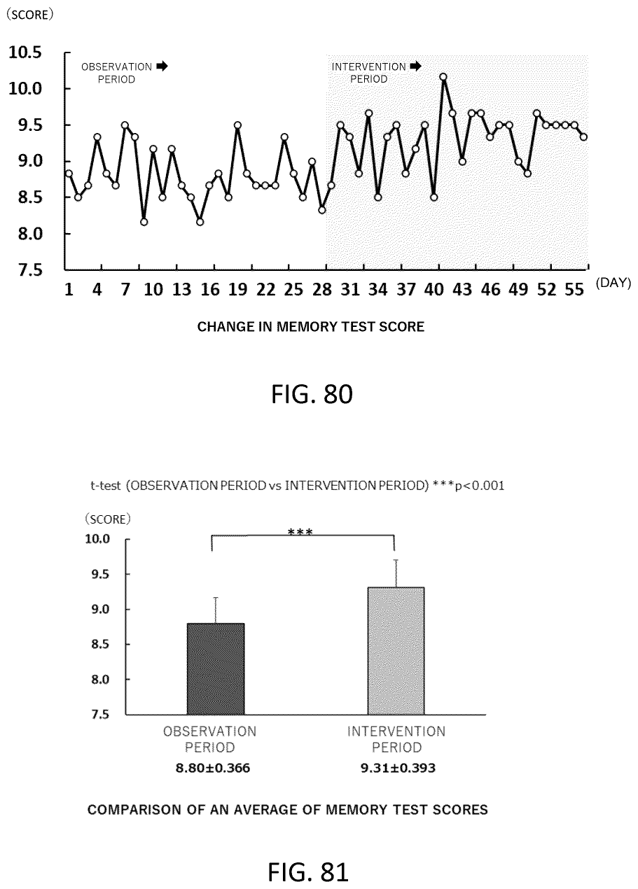

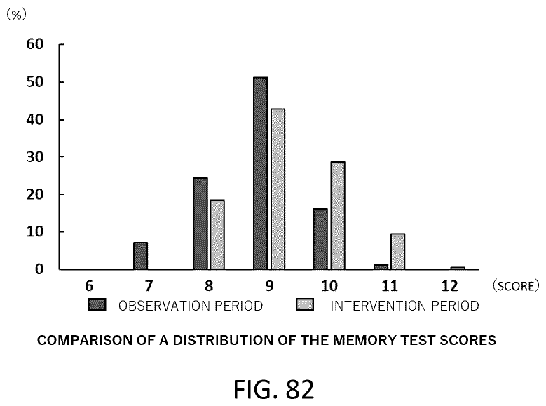

A change in score of the memory test in the observation period and the demonstration period is illustrated (FIG. 80). An increase in memory test score was not confirmed in the observation period, but a tendency toward an increase in score was confirmed in the demonstration period, and a score average in the observation period was 8.80 points and a score average in the demonstration period was 9.31 points, which means that the score average significantly increased (p<0.001) (FIG. 81). In addition, score distribution comparison in each period was performed, and 7 points were obtained 12 times (7.1%) and 0 times (0%), 8 points were obtained 41 times (24.4%) and 31 times (18.5%), 9 points were obtained 86 times (51.2%) and 72 times (42.9%), 10 points were obtained 27 times (16.1%) and 48 times (28.6%), 11 points were obtained 2 times (1.2%) and 16 times (9.5%), and 12 points were obtained 0 times (0%) and 1 time (0.6%) in the observation period and the demonstration period, respectively, and improvement in test score was confirmed (FIG. 82).

### SUMMARY

In the second example, for one healthy adult male, factors that improve and deteriorate the memory were specified by analyzing data of the daily intake nutrient amount, the memory test score, and the number of memory tests, and memory improvement was verified by designing and ingesting a meal in which these factors are adjusted.

In the second example, the model formula for predicting the memory test score from the intake nutrient amount and the number of memory tests was derived by performing the multiple regression analysis on vital data during the test period. The analysis was performed seven times in total, and the average multiple correlation coefficient of the model formula was 0.776±0.100. Since a high correlation was observed in each analysis, it was suggested that the memory test score can be predicted by the model formula. In addition, the types of the memory improvement factor and the memory deterioration factor included in the model formula and the degree of influence of each nutrient on the objective variable were different for each analysis, and for a certain nutrient, the coefficient sign was reversed. The data used was different for each analysis, and the latest data at each analysis time point was used. From this, it is inferred that a physical condition at the time of analysis is reflected, and it is considered that it is possible to design a meal with high accuracy according to more recent data.

Although different nutrients are included in each model formula, there are nutrients adopted at a high frequency, and it is considered that such nutrients have a large influence. Iron as the improvement factor has an ability to carry oxygen, which is important for the brain function, but it has been found that an absorption rate thereof is as low as about 15%. It is conceivable that iron is shown as the improvement factor to compensate for such a low absorption rate. Lipid as the improvement factor is an important nutrient constituting 60% of the brain. The main energy source in the brain is glucose, but when glucose is insufficient, ketone bodies are created from lipids in the liver to always perform regulation so as to prevent the energy from being insufficient. In addition, lipid has a function as a component of a cell and relates to information transmission. It is considered that such a function relating to the brain function. The salt equivalent as the deterioration factor indicates the amount of sodium. Sodium moves in and out of the cell by active transport, creating a concentration gradient. Various substances such as sugars and amino acids are transported depending on the concentration gradient. The active transport by sodium also relates to maintaining an ionic environment inside the cell, and such a function may relate to the brain function. Among the nutrients included in the model formula, those in which improvement and deterioration were reversed during the test period were also confirmed. Such a nutrient is mineral which is an important nutrient relating to various metabolism of the human body. Minerals and vitamins are often shown as related factors in prior studies of the optimal personal diet, and may greatly relate to a physical activity function, and it is considered that improvement and deterioration are reversed since the required amount always changes depending on the physical condition.

The model formula may include the number of memory tests (Analysis No. 1, Analysis No. 5, and Analysis No. 6), and it was possible to adjust not only the nutrient amount but also the number of memory tests. From this, an influence of a non-nutrient factor can also be considered, and it is considered that it is possible to design an optimized menu in combination with nutrient adjustment.

In order to show the degree of adjustment of the nutrient amount and the number of tests of the optimal personal diet designed for each analysis, the degree of adjustment of the nutrient was calculated and compared. In the second example, the memory improvement factor was adjusted to increase in all nutrients compared to the observation period as in the previous study. Adjustment was made to basically decrease the deterioration factor, but adjustment was made such that the deterioration factor was increased in some nutrients, or the deterioration factor was almost unchanged. It is conceivable that the optimal personal diet can be designed, and a lifestyle, a taste preference, and the like of an individual can be considered by performing adjustment such that “the effect by the sum of the improvement factors exceeds the effect by the sum of the deterioration factors”.

In the second example, analysis including an amino acid as a nutrient other than 20 nutrients was performed. As a result, there was a high correlation between amino acids, and as a result, the VIF was 10 or more, and reliability of the model formula was greatly lowered. Therefore, the amino acid was excluded from the variables. However, in a case where the amino acid is included in the model formula without any problem, nutrient adjustment can be performed by using a supplement or the like, and an appropriate optimal personal diet can be designed.

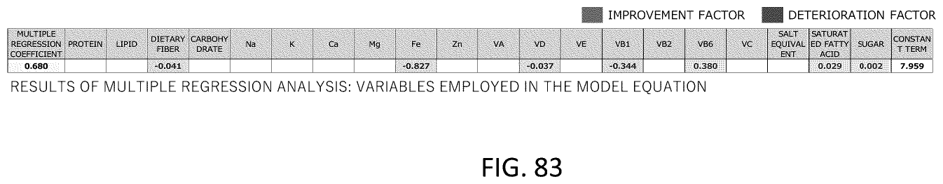

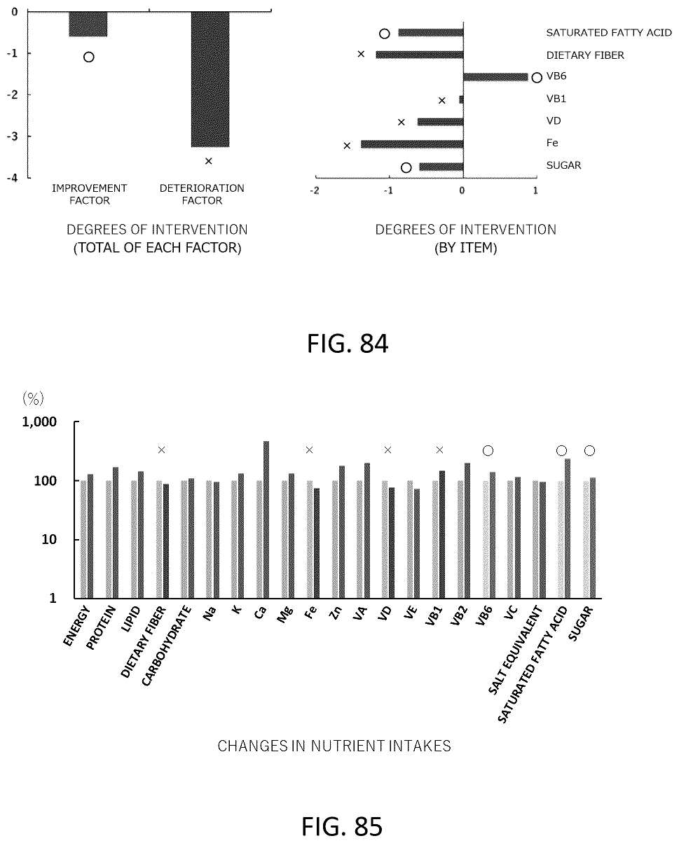

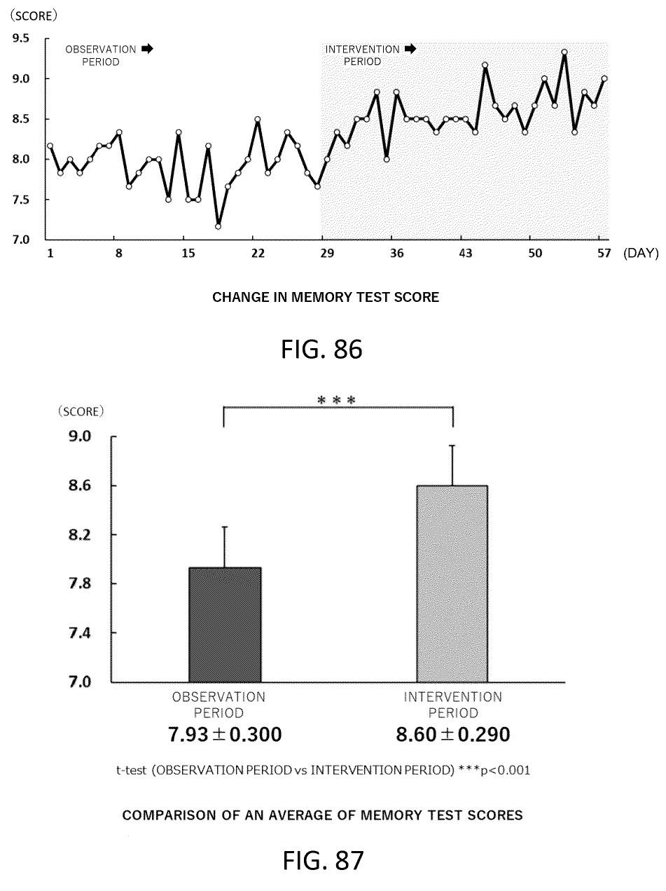

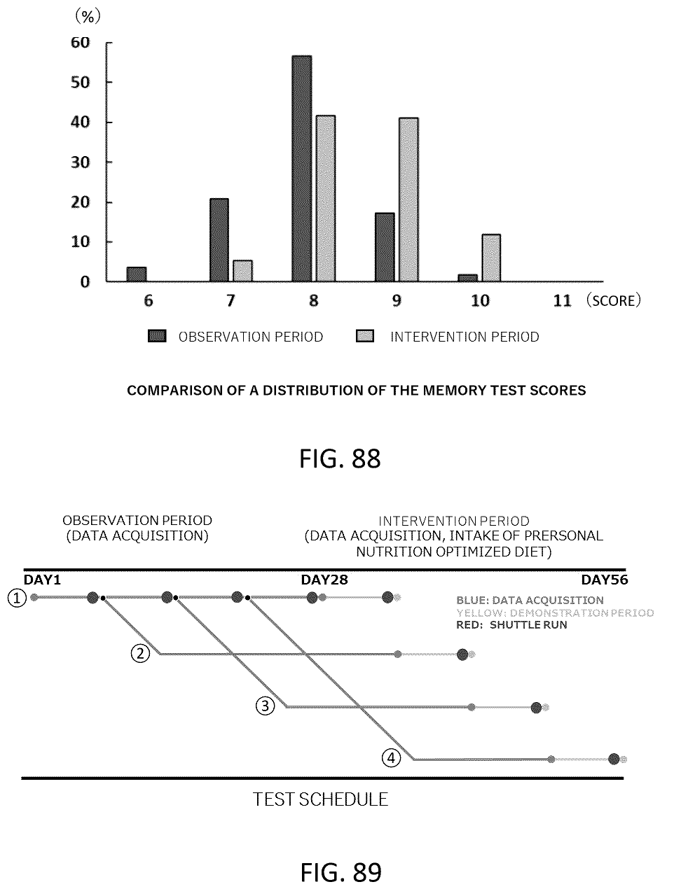

A preliminary test was performed to examine the conditions of the second example. In the preliminary test, ingested nutrients were analyzed, and the optimal personal diet was designed and ingested by adjusting nutrients as the memory improvement factors and the memory deterioration factors. In the preliminary test, a post-observation period in which the optimal personal diet was not ingested was provided after the demonstration period, and the memory test result was shown in a “supplement data preliminary test result”. As compared with the observation period in which there was no tendency for the memory test score to increase, there was a tendency for the score to increase in the demonstration period, and the score average significantly increased (p<0.001). However, there was no tendency for the score to increase in the post-observation period, and no significant score average increase was confirmed as compared with the demonstration period (FIGS. 83 to 88). From this, it is considered that the score increase confirmed in the demonstration period is due to nutrient adjustment and is not a placebo effect.

The analysis method used in the second example is not limited to using nutrients, and those that are quantifiable can be incorporated into the model formula. Therefore, it is possible to promote an improvement effect from a viewpoint other than nutrients and to apply the analysis method to imp of various health indices. In addition, since the analysis is performed on the basis of data of one individual, it is possible to provide a personalized instruction that maintains the individual's physical constitution and the QOL, unlike the conventional instruction for the public. Therefore, the optimal personal diet holds the potential to become a new form of healthcare tailored to an individual.

### Third Example

In a third example, a cardiopulmonary function improvement effect by optimization of an intake nutrient amount was verified for the purpose of improving endurance of an individual. The resting heart rate was used as an index of the cardiopulmonary function. The resting heart rate changes depending on diet or exercise habits, and the endurance is improved by lowering the resting heart rate. In addition, in order to improve the improvement effect, introduction of the amount of exercise as a factor other than the intake nutrient amount was also examined.

### Test Material and Method

### Test Material

### Test Subject

### 1) One Healthy Adult Male

Three meals were taken per day during the test period. Ingestion of health foods, supplements, and alcohols and ingestion of meals other than three meals (additional meals and snacks) were prohibited, and beverages were limited to water only. In addition, irregular life was avoided, and strenuous exercise other than designated exercise was not performed.

### 2. Experimental Equipment

1) Body weight and composition monitor (HBF-255T, Omron Healthcare Co., Ltd., Kyoto) 2) Wearable device (Fitbit Charge3, Fitbit (Google), USA)
3) Smartphone (iPhone (registered trademark) XR, Apple, USA)
4) Pulse oximeter (yuwell pulse oximeter YX301, Glox Inc., Tokyo)
5) Physical test 20-m shuttle run sound (the Ministry of Education, Culture, Sports, Science and Technology)

### 3. Software Application

1) OMRON connect (Omron Healthcare Co., Ltd., Kyoto)
2) Asken Diet (asken Inc., Tokyo)
3) Fitbit (Fitbit (Google), California, USA)
4) SPSS Statistics ver. 25 (IBM, New York, USA)

### Test Method

### Test Schedule

The test period was divided into two periods, and the test was performed based on a schedule (FIG. 89) of an observation period (28 days) and a demonstration period (28 days). The demonstration period was divided into first to fourth stages, and multiple regression analysis was performed every week to create a resting heart rate prediction model formula. In the observation period, the intake nutrient amount of three meals a day and the amount of exercise (described on the next page) were used as data used for analysis, and were recorded. In the demonstration period, data acquired in the observation period was subjected to multiple regression analysis, and an optimal personal diet obtained by adjustment of nutrients and the amount of exercise based on the analysis result was taken as three meals (breakfast, lunch, and dinner) and the exercise was performed and recorded. In addition, in order to evaluate the endurance, a 20-m shuttle run was performed every seven days, and comparison was made before and after the observation period and the demonstration period, and improvement in endurance for each analysis was confirmed.

### Measurement Method for Each Data

### Resting Heart Rate

The resting heart rate as an objective variable was measured using a wearable device.

### Intake Nutrient Amount

During the observation period, nutrient estimation and recording were performed by photographing three meals a day using a smartphone application for meal management. Recording targets were an intake of a total of 20 nutritional components of protein, lipid, sugar, a total dietary fiber content, carbohydrate, sodium, potassium, calcium, magnesium, iron, zinc, vitamin A, vitamin D, vitamin E, vitamin B1, vitamin B2, vitamin B6, vitamin C, equivalent salt, and saturated fatty acid, and an intake energy amount from an ingested meal, and the total amount per day was recorded. A model formula for predicting the resting heart rate from the daily nutrient amount was created. Data based on the Standard Tables of Food Composition in Japan (eighth edition, 2020) revised in December 2020 is used.

### Amount of Exercise

In this test, a change in amount of exercise was used as an explanatory variable in addition to the intake nutrient amount, and a time during which aerobic exercise (jogging) that affects the resting heart rate was performed was taken as the amount of exercise. In the observation period, the exercise was performed for a time randomly selected from 0 minute, 15 minutes, 30 minutes, and 45 minutes once a day at a constant speed of 12 km/h (0 min: 7 times, 15 min: 6 times, 30 min: 7 times, and 45 min: 8 times). In addition, in order to eliminate a decrease in resting heart rate due to the influence of jogging, jogging was performed in the same manner as in the observation period from one month before the start of this test.

### Derivation of Model Formula

Multiple regression analysis according to SPSS Statistics ver. 25 (IBM, New York, USA) was used to derive a model formula for designing the optimal personal diet. The objective variable was the daily resting heart rate, the explanatory variables were the amount of exercise and the intake nutrient amount of the previous day, and an analysis method by a backward elimination method was used. The model formula for predicting the resting heart rate by seven elements was constructed from the analysis result, and a nutrient having a positive coefficient was defined as a resting heart rate decreasing factor (improvement factor), and a nutrient having a negative coefficient was defined as a resting heart rate increasing factor (deterioration factor).

### Design of Optimal Personal Diet

A target prediction point of the resting heart rate was set, adjustment was performed to increase the improvement factor clarified by the derived model formula and decrease the deterioration factor, and the optimal personal diet capable of achieving the prediction point was designed for three meals a day. In order to maintain the QOL of a practitioner, the optimal personal diet was created in consideration of a balance of main dishes, side dishes, and the like, similarly to the meal taken in the observation period. In addition, when a jogging time was the improvement factor, the jogging was performed for 45 minutes except for a date for the shuttle run. When the jogging time was not adopted in the model formula, jogging was performed at random time similarly to the observation period.

### Endurance Evaluation Item

### 20-m Shuttle Run (Physical Test, the Ministry of Education, Culture, Sports, Science and Technology)

In the field of education, the 20-m shuttle run used as an endurance measurement method was performed to evaluate the endurance. In order to reduce an influence of the improvement in endurance due to the 20-m shuttle run, measurement was performed once every seven days, and measurement was performed four times in each of the observation period and the demonstration period. In addition, in order to eliminate technical improvement factors due to repetition, measurement was performed one month before the test, and this test was conducted after improvement of the record was not recognized.

### Blood Oxygen Level

A blood oxygen level at the end of daily jogging was measured. The blood oxygen level is a value obtained by examining (percutaneously) a binding ratio of hemoglobin included in red blood cells flowing in blood (arterial blood) supplied from the heart to the whole body with oxygen through the skin, and is also used for evaluation of the cardiopulmonary function. Therefore, the improvement of the endurance was confirmed by comparing before and after the observation period and the demonstration period.

### Statistical Processing

### Significant Difference Test

For the measurement results, an average value and a standard deviation were calculated for each observation period and each demonstration period. Physical changes corresponding to an improved diet were reflected in the following day, and thus, test data on the first day of the demonstration period was excluded. For comparison between the observation period and the demonstration period, statistical processing was performed by an unpaired t-test. A significance level in the test was 5% or less in a two-sided test (*p<0.05, **p<0.01, and ***p<0.001).

### Nutrient Adjustment Degree Evaluation

The intake nutrients were standardized, and the degree of adjustment of each nutrient was calculated and evaluated. The standardization is to perform scaling such that an average of data becomes 0 and a variance becomes 1, and the standardization enables comparison of nutrients with different units. The average and standard deviation of the nutrient data used in the analysis were used for standardization, and the value obtained by subtracting the standardized nutrient amount in the observation period from the average of the standardized nutrient amount in the demonstration period was multiplied by the absolute value of the normalization coefficient derived from the multiple regression analysis to obtain the degree of adjustment. The degree of adjustment was calculated for each degree of adjustment of each resting heart rate related factor. When the value of the degree of adjustment is positive, the adjustment is performed more than the observation period, and when the value is negative, the adjustment is performed less than the observation period.

### Test Result

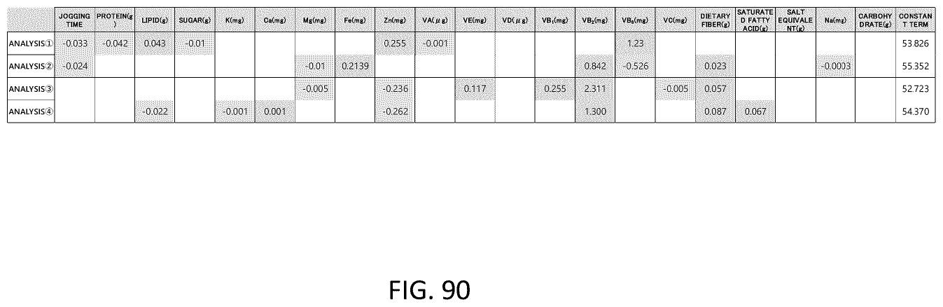

Specification of Cardiopulmonary Function Related Factor Multiple regression analysis was performed using SPSS Statistics ver. 25 with the resting heart rate acquired during the observation period as an objective variable and the intake nutrient amount and the amount of exercise (jogging time) as explanatory variables. Based on the obtained multiple regression coefficient, the model formula for predicting the resting heart rate was constructed, and the resting heart rate related factor was specified from the sign of the coefficient (FIG. 90). An average value of the multiple correlation coefficients of four analyses was 0.835, and a high correlation was observed between the intake nutrient and the measured value of the resting heart rate, suggesting that the resting heart rate can be predicted from the intake nutrient amount and the amount of exercise (jogging time).

$$Analysis data : Oct . 5 , 2021 to Oct . 29 , 2021 ( 25 days ) R = 0.86$$

$$Resting heart rate ( bpm ) = - 0.033 [ jogging time minute ] - 0.042 [ protein g ] + 0.043 [ lipid g ] - 0.01 [ sugar g ] + 0.255 [ zinc mg ] - 0.001 [ vitamin A µg ] + 1.23 [ vitamin B6 mg ] \qquad Analysis 1$$

$$Analysis data : Oct . 12 , 2021 to Nov . 5 , 2021 ( 25 days ) R = 0.84$$

$$Resting heart rate ( bpm ) = - 0.024 [ jogging time minute ] - 0.01 [ magnesium mg ] + 0.213 [ iron mg ] + 0.842 [ vitamin B2 mg ] - 0.526 [ vitamin B6 mg ] + 0.023 [ dietary fiber amount g ] - 0.000259 [ sodium mg ] \qquad Analysis 2$$

$$Analysis data : Oct . 19 , 2021 to Nov . 12 , 2021 ( 25 days ) R = 0.83$$

$$Resting heart rate ( bpm ) = - 0.005 [ magnesium mg ] - 0.236 [ zinc mg ] + 0.117 [ vitamin E mg ] + 0.255 [ vitamin B1 mg ] + 2.31 [ vitamin B2 mg ] - 0.005 [ vitamin C mg ] + 0.057 [ total dietary fiber content g ] \qquad Analysis 3$$

$$Analysis data : Oct . 26 , 2021 to Nov . 19 , 2021 ( 25 days ) R = 0.81$$

$$Resting heart rate ( bpm ) = - 0.022 [ lipid g ] - 0.001 [ potassium mg ] + 0.001 [ calcium mg ] - 0.2 \; 62 [ zinc mg ] + 1.3 [ vitamin B2 mg ] + 0.087 [ to tal dietary fiber content g ] + 0.067 [ saturated fatty acid g ] \qquad Analysis 4$$

### Design of Optimal Personal Diet

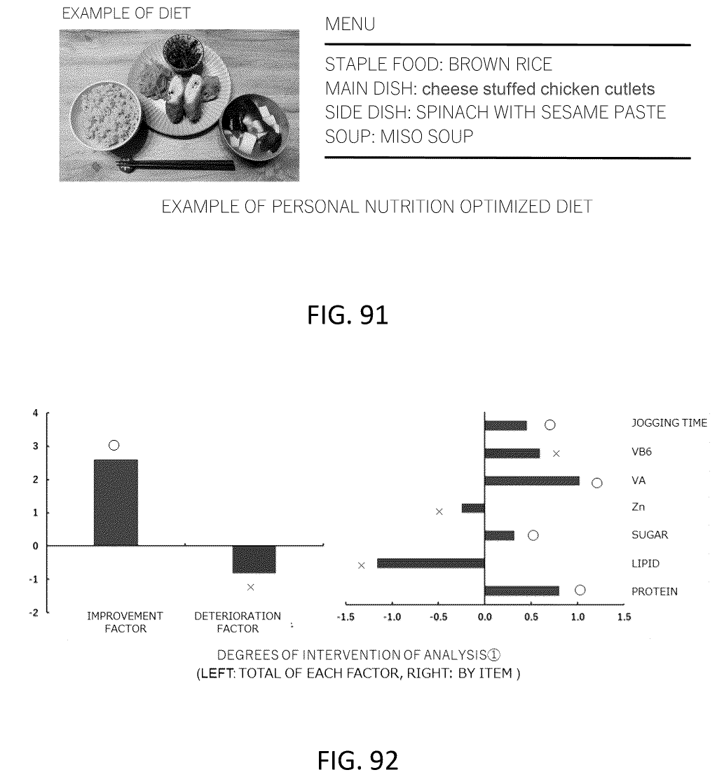

A target prediction value was set based on the resting heart rate in the observation period, and the optimal personal diet (three meals a day) that can achieve the target prediction value was designed. A predicted resting heart rate was set to 50 (bpm), and a menu was created by adjusting ingredients. An example of the optimal personal diet is an example of Analysis 2 and is illustrated in (FIG. 91). Since the jogging time was specified as the improvement factor in Analysis 1 and Analysis 2, the jogging time was adjusted to be increased from an average of 23.6 minutes in the observation period to an average of 30.0 minutes in the demonstration period (0 min: 6 times, 15 min: 4 times, 30 min: 2 times, and 45 min: 16 times).

### Degree of Adjustment of Nutrient

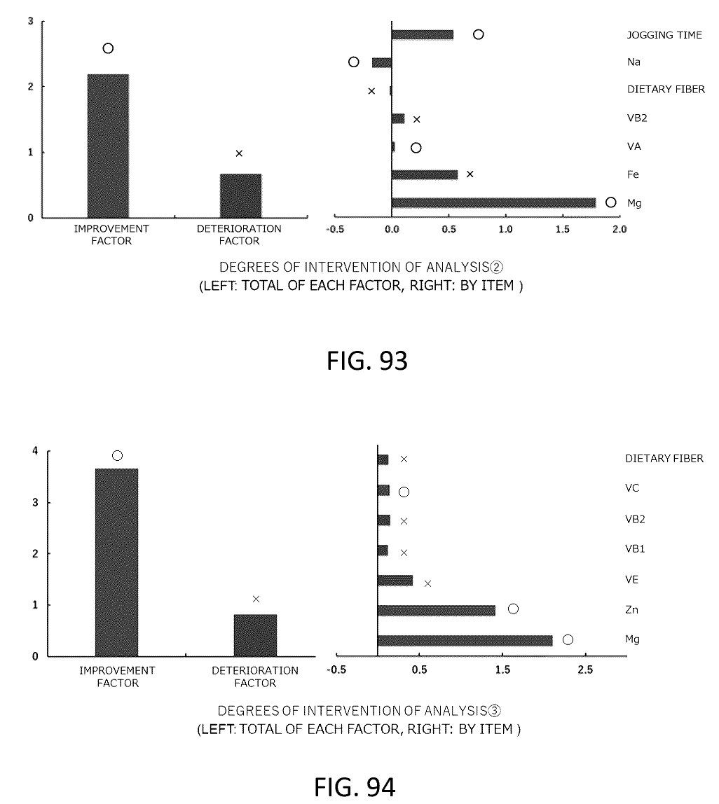

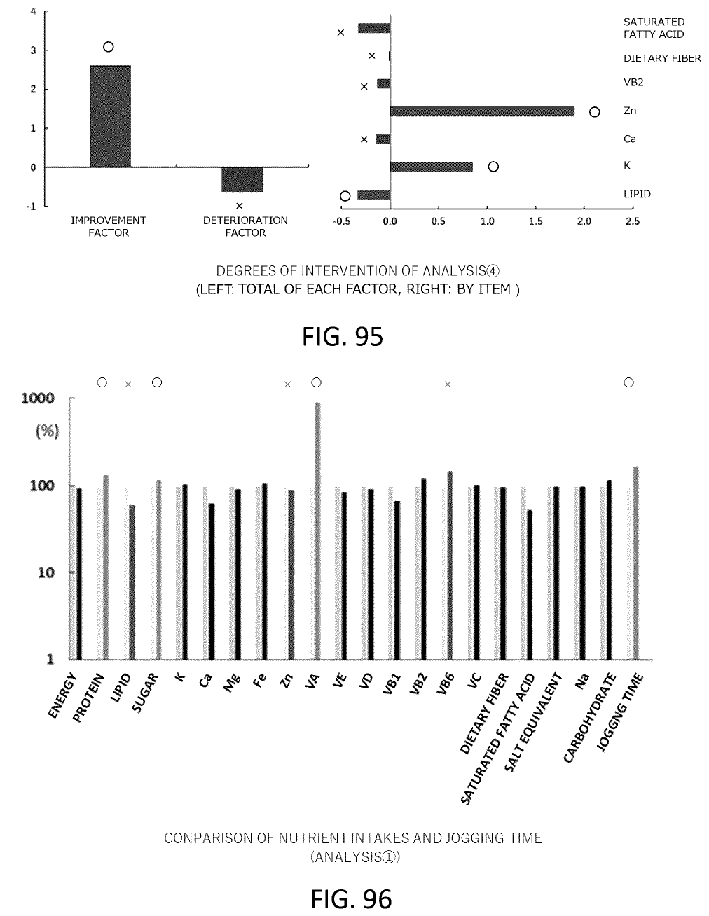

The overall degree of adjustment and the individual degree of adjustment of the improvement factor and the deterioration factor taken in the demonstration period are shown below (FIGS. 92 to 95). The overall degree of adjustment of the improvement factors was a positive value in all the analyses, but the overall degree of adjustment of the deterioration factors was a negative value in Analysis 1 and Analysis 4 and was a positive value in Analysis 2 and Analysis 3. In the design of the optimal personal diet, since it is the most ideal to adjust the improvement factor to a positive value and adjust the deterioration factor to a negative value, results as in Analysis 1 and Analysis 4 were obtained. However, in order to maintain the QOL of an individual, it is important to reflect a preference of food and not to make a large change in normal dietary life, and there are cases where it is not possible to reduce ingredients including the deterioration factors. Under such circumstances, adjustment is performed to increase or change the ingredient containing more improvement factors, so that the design is performed to achieve the target value. In addition, there were ingredients that include many deterioration factors, but the total of improvement factors was even greater. In this test, brown rice corresponds to such an ingredient, and Analysis 2 and Analysis 3 used brown rice, so that the deterioration factor was a positive value.

### Resting Heart Rate Related Nutrient Amount

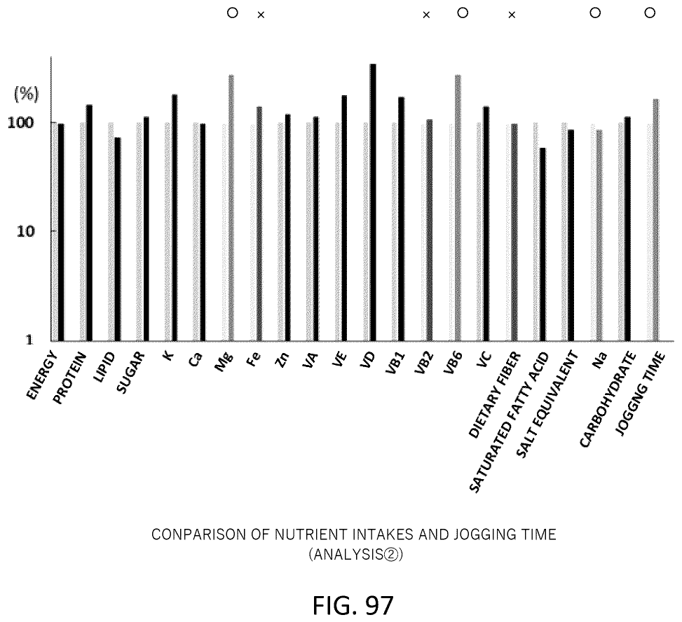

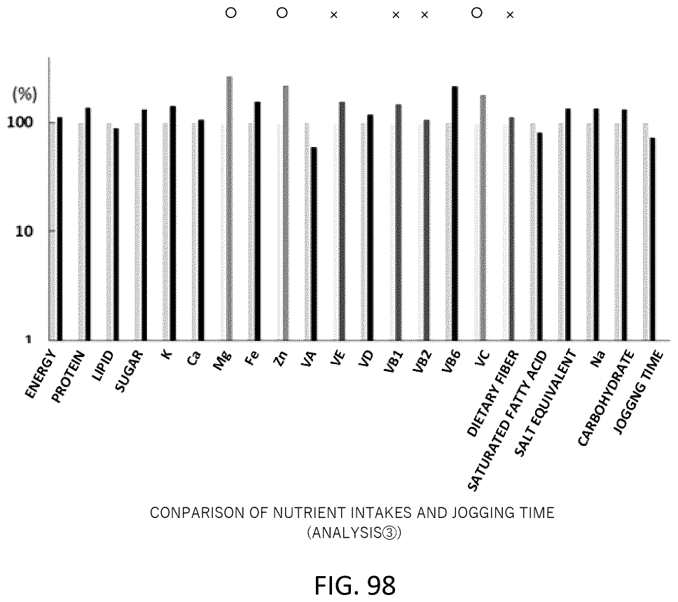

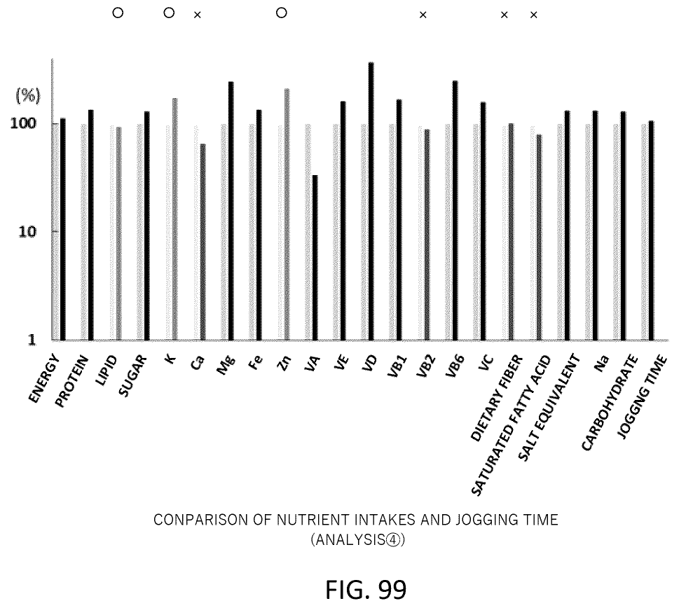

Graphs illustrating comparison of the amounts of energy and nutrients ingested by the test subject from the test food during the demonstration period with the intakes during the observation period as 100% are illustrated in FIGS. 96 to 99 below. In two bar graphs with the same label, the left bar graph indicates the intake during the observation period, the right bar graph indicates the intake during the demonstration period, a circle mark indicates the resting heart rate improvement factor, a cross mark indicates the resting heart rate deterioration factor, and the energy amount was changed within ±10% as compared with the average in the observation period. In Analysis 3, the jogging time in the demonstration period was 28% shorter than the jogging time in the observation period. As described in the following section, since the resting heart rate decreased even within the period of Analysis 3, it was suggested that the influence of the nutrient amount was large.

### Change in Resting Heart Rate

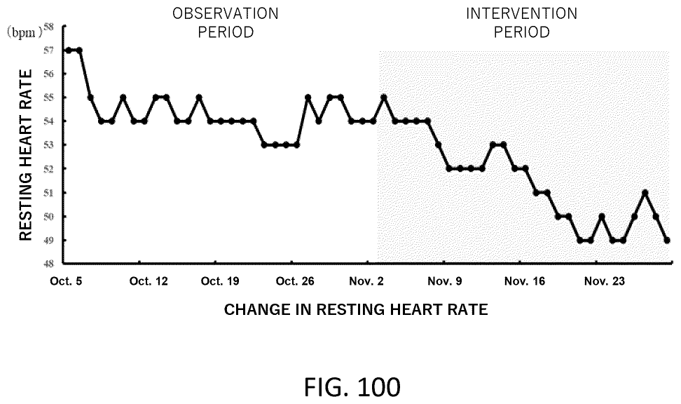

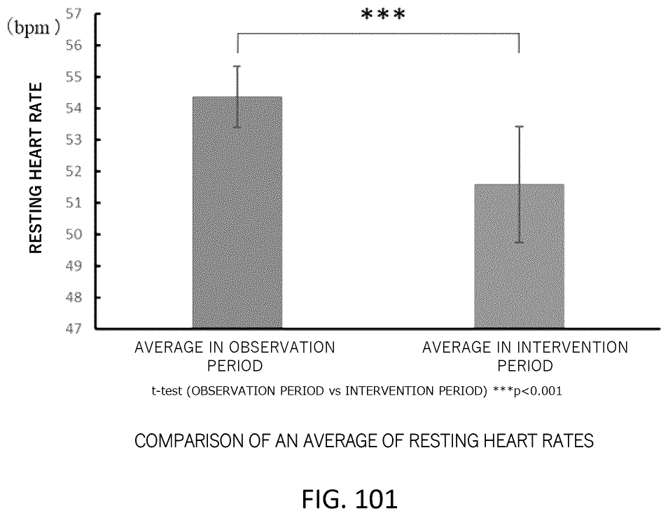

Changes in resting heart rate during the observation period and the demonstration period are illustrated (FIG. 100). In addition, the average in the observation period and the average in the demonstration period were illustrated (FIG. 101). As compared with the average of 54.4+0.97 (bpm) in the observation period, the resting heart rate was significantly decreased to 51.6+1.84 (bpm) in the demonstration period (p<0.001).

### Motor Function Test Result

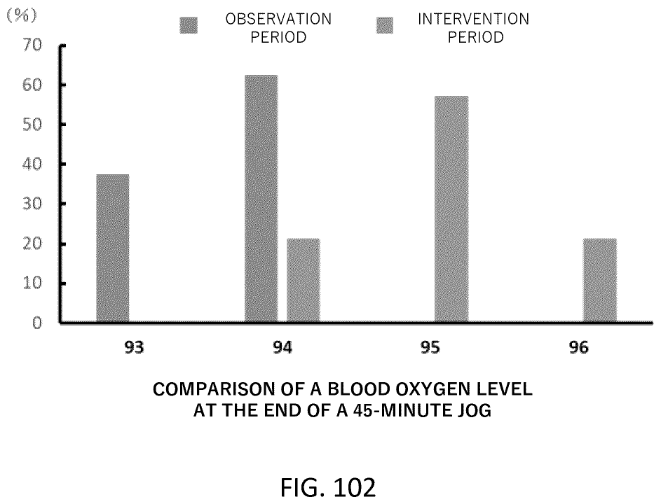

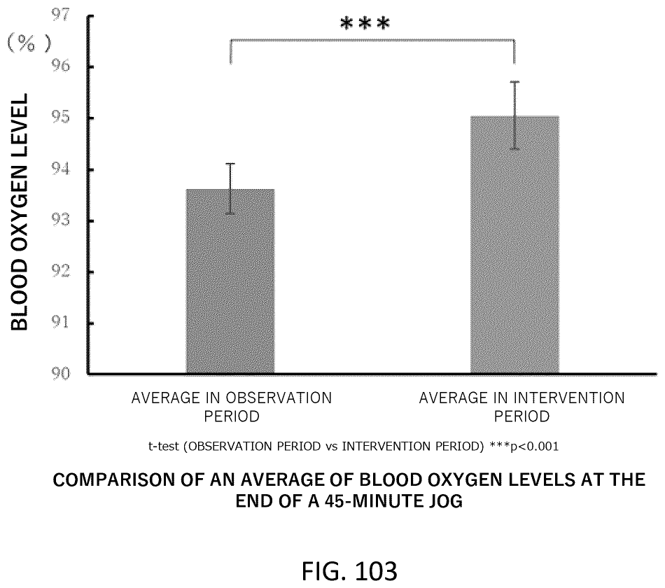

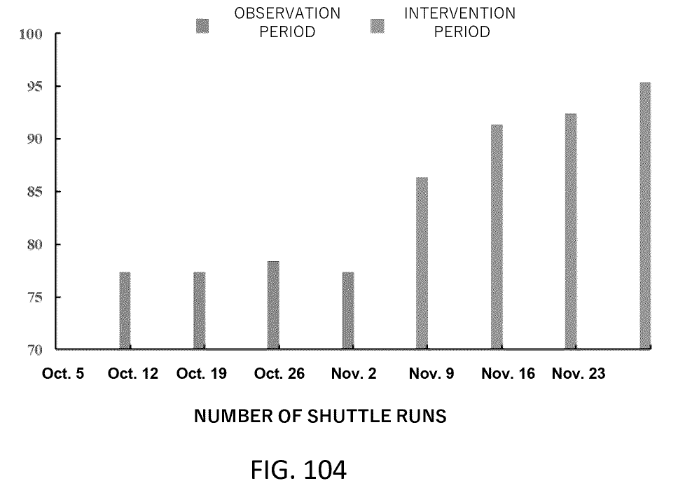

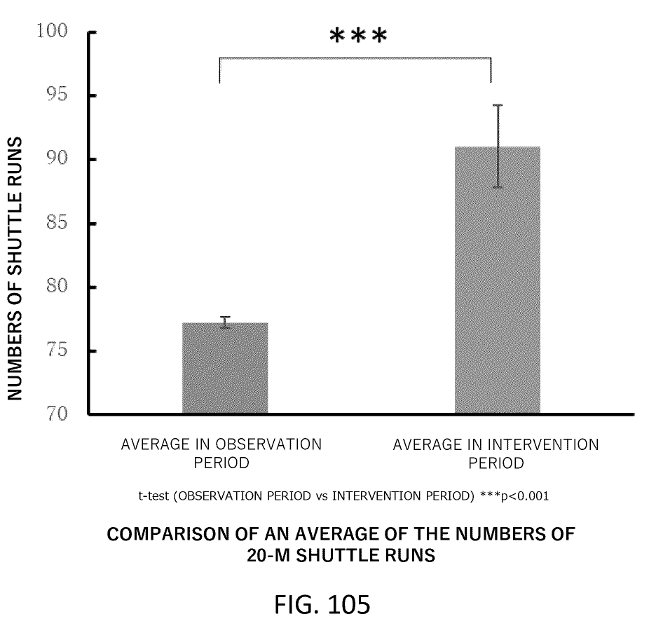

A percentage of the blood oxygen level at the end of a 45-minute jog is illustrated (FIG. 102). The average in the observation period was 93.6±0.48%, whereas the average in the demonstration period was 95.1±0.66%, which means that the blood oxygen level was significantly increased (p<0.001) (FIG. 103). The result of the 20-m shuttle run is illustrated (FIG. 104). An increase from 75 times in the observation period to 95 times in the demonstration period was shown. In addition, the result of the 20-m shuttle run was significantly increased from the average of 77.3 times in the observation period to the average of 91.0 times in the demonstration period (p<0.001) (FIG. 105).

### SUMMARY

In a third example, a cardiopulmonary function improvement effect by optimization of an intake nutrient amount was verified for the purpose of improving endurance.

It was suggested from the results of this test that it is possible to reduce the resting heart rate by optimizing the intake nutrient amount and exercise of an individual, and it is possible to predict the resting heart rate. Regarding results of the evaluation items for endurance, the result of the blood oxygen level at the end of the 20-m shuttle run and jog was also significantly increased (p<0.001). From this, it was suggested that the endurance can be estimated from the resting heart rate of an individual.

In addition, by incorporating the amount of exercise into the analysis, it is possible to determine the amount of exercise suitable for an individual's life and then design a meal in accordance with the determined amount of exercise. Furthermore, it is shown that a quantifiable index such as the amount of exercise can be incorporated into the analysis, and from this, it is considered that an influence of external factors such as stress and sleep, which cannot be considered in this test, can be considered.

In this test, in order to cope with daily changes of the environment in the body, a continuous analysis method in which analysis is performed once a week was adopted. In each analysis result, all elements of the model formula are different, and it is inferred that a physical condition at the time of analysis is reflected, and it is considered that it is possible to design a meal with high accuracy according to more recent data. The jogging time closely relating to the resting heart rate was not reflected in the model formula in Analysis 3 and Analysis 4. This indicates that the influence of the jogging time is lower than that of the seven selected nutrients, and it was suggested that the influence of the adjustment of nutrients is greater than the increase in amount of exercise depending on the individual's physical condition. In this test, a factor having the highest degree of influence of improvement was magnesium. It has been shown that a higher serum magnesium level results in a significantly lower risk of cardiovascular disease and a higher dietary magnesium intake (up to about 250 mg per day) results in a significantly lower risk of ischemic heart disease caused by a reduced blood supply to the myocardium. Therefore, it is considered that magnesium relates to enhancement of the cardiopulmonary function also in this test. In addition, mineral had a high frequency of becoming the deterioration factor. Mineral may greatly relate to a physical activity function, and the required amount of mineral always changes depending on the physical condition and purpose. Therefore, in this test, it was considered that the resting heart rate of the test subject may have exceeded the required amount.

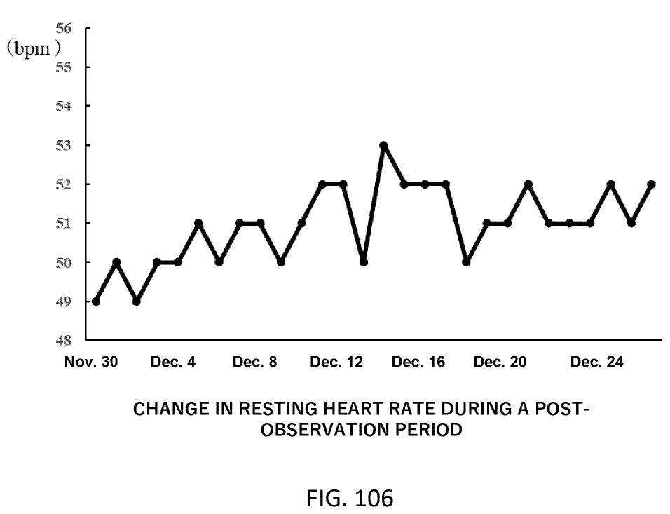

Since the amount of exercise is increased in the demonstration period as compared with the observation period, a post-observation period was provided after the demonstration period in order to consider a possibility that a decrease in heart rate (a change in endurance) is a result associated with an increase in amount of exercise. In the post-observation period, the same amount of exercise as in the demonstration period was set, and ingestion of the optimal personal diet was not performed. As a result, no significant change was observed when the average values of the resting heart rates in the demonstration period and the post-observation period were compared (FIG. 106), suggesting that the influence of the optimal personal diet was strong in the demonstration period.

This test suggested that the cardiopulmonary function can be improved by personal optimization of diet and exercise. This method is expected to predict and improve not only the cardiopulmonary function but also any quantifiable index, and thus has the potential to become a new healthcare technology that can improve various health indexes.

### Second Embodiment

In the first embodiment described above, a prediction model is created by analyzing a correlation between a measured value linked to a goal to be achieved and data (ingested nutrients, activities, life stages, or the like) acquired for a user himself/herself, and an improvement factor and/or a deterioration factor is specified. In a second embodiment, the user specifies the improvement factor and/or the deterioration factor for the user by using data and/or an analysis result of another user.

Hereinafter, a method of suggesting a meal menu for the user by using a measured value linked to nutrients and a goal to be achieved acquired for another user and/or a nutrient model analyzed for another user will be described.

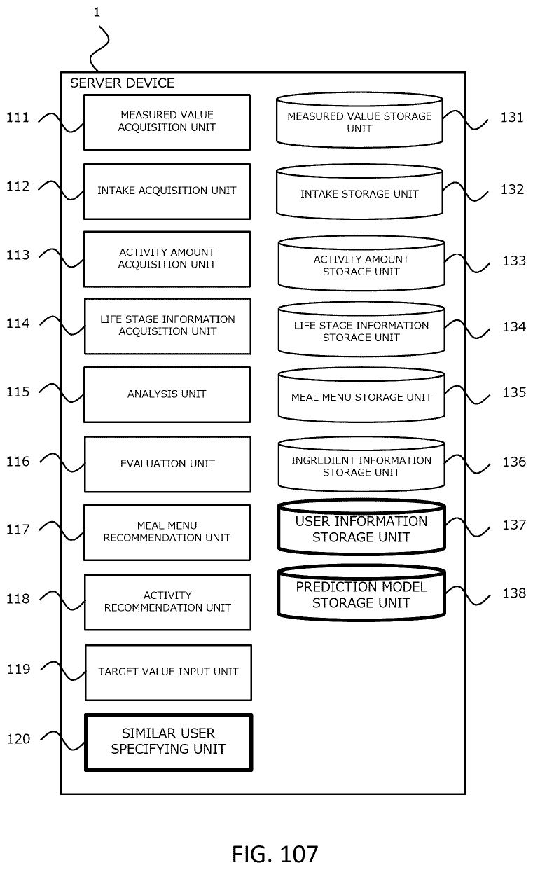

FIG. 107 is a diagram illustrating a functional configuration example of a server device 1 according to the second embodiment. As illustrated in the drawing, in the second embodiment, the server device 1 further includes a user information storage unit 137 that stores information regarding the user (hereinafter, referred to as user information), a prediction model storage unit 138, and a similar user specifying unit 120.

The prediction model storage unit 138 stores a prediction model (a nutrition model, a nutrition-activity model, a nutrition-life stage model, a nutrition-date and time model, or a nutrition-combination model) created by an analysis unit 115. The prediction model storage unit 138 stores information including the prediction model (hereinafter, referred to as prediction model information). The prediction model information can include information indicating the goal to be achieved by the user (hereinafter, referred to as goal data, and the goal data may be, for example, text data such as “diet” and “lower blood pressure”), information indicating a measured value relating to the goal to be achieved (hereinafter, referred to as measured value specifying information, and for example, the measured value specifying information can be text data such as “body weight” and “blood pressure” or a label value), and the prediction model (the nutritional model, the nutrition-activity model, the nutrition-life stage model, the nutrition-date and time model, the nutrition-combination model, or the like) in association with information for specifying the user (for example, a user ID).

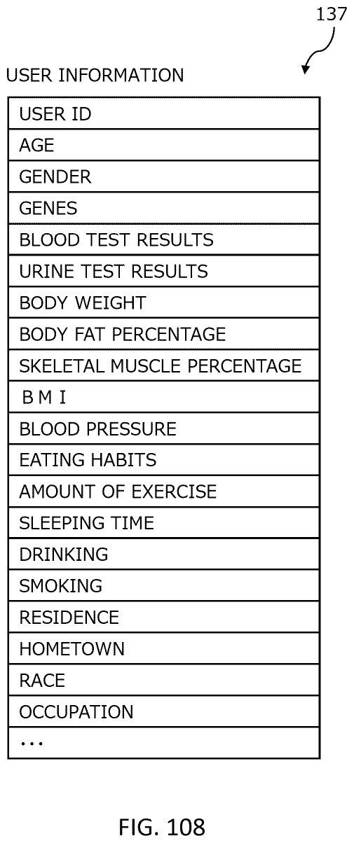

FIG. 108 is a diagram illustrating a configuration example of the user information stored in the user information storage unit 137. The user information includes various attributes relating to the user in association with information (user ID) for specifying the user. The attributes may include, for example, age, gender, genes, blood test results, urine test results, biomarkers (a body weight, a body fat percentage, a skeletal muscle percentage, BMI, a blood pressure, or the like), lifestyle (eating habits, the amount of exercise, a sleeping time, drinking, smoking, or the like), residence, hometown, race, and occupation. The dietary life can include a result (an option prepared in advance may be selected or free text data may be used) of a questionnaire for the user about a preference for food (like/dislike), an intake nutrient composition, an intake calorie amount, and the like.

In the second embodiment, data and/or analysis result of another user (hereinafter, referred to as a similar user) having similar attributes to the user are used. The similar user specifying unit 120 specifies the similar user having similar attributes to the user. The similarity of the attributes of the users can be obtained by a known method. For example, the degree of similarity can be obtained by standardizing and summing the degree of matching of numerical values, the degree of matching of texts, and the like.

There are the following four patterns in a method of using the data and/or analysis result of the similar user.
Pattern 1: Using an analysis result of another similar user
Pattern 2: Using an analysis result obtained by analyzing data of another similar user
Pattern 3: Using an analysis result obtained by analyzing data of plurality of other similar users
Pattern 4: Using analysis results of a plurality of other similar users Each pattern will be described below.

### <Pattern 1: Using Analysis Result of Another User>

In Pattern 1, the analysis result of one similar user is used for the user as it is.

The similar user specifying unit 120 can specify, as the similar user, a user having the most similar attributes from among other users having the same goal to be achieved as the goal to be achieved by the user. The similar user specifying unit 120 can receive an input of the goal to be achieved from the user, search the prediction model storage unit 138 for the prediction model information matching the received goal to be achieved (or similar to a predetermined degree or more), acquire the user information corresponding to the user ID included in the searched prediction model information from the user information storage unit 137, determine the similarity between the attributes included in the acquired user information and the attributes included in the user information corresponding to the user, and specify the user information having the highest similarity (user information other than itself).

The analysis unit 115 can acquire the prediction model created for the similar user (and the improvement factor and the deterioration factor which are explanatory variables of the prediction model) from the prediction model storage unit 138, and set the prediction model and the improvement factor and the deterioration factor for the user.

An evaluation unit 116 can evaluate a meal menu on the basis of the improvement factor and the deterioration factor of the similar user, and a meal menu recommendation unit 117 can select the meal menu according to the evaluation value or adjust nutrients such that the measured value is improved.

In the case of using a dimensionless index in which the measured value is represented by a ratio of a plurality of measured values, a coefficient can be corrected when using the prediction model of the similar user. For example, in a case where the measured value is BMI, the BMI is calculated from the body weight and the height, and can be corrected by a difference in weight. For example, in a case where data of the similar user with the BMI=26 and the body weight=60 kg is adapted to the user with the BMI=26 and the body weight=80 kg, correction can be made with (80÷60)×coefficient. In this manner, the evaluation unit 116 can evaluate the meal menu by multiplying the coefficient by a ratio of the attribute of the similar user to the attribute of the user for the attribute (other measured value) correlated with the measured value.

### <Pattern 2: Using Analysis Result Obtained by Analyzing Data of Another User>

In Pattern 2, data acquired for one similar user is analyzed.

The similar user specifying unit 120 can specify one user having the most similar attributes as the similar user from among other users who have registered the measured value relating to the goal to be achieved by the user in the measured value storage unit 131.

The analysis unit 115 can reads the measured value and the intake of nutrients corresponding to the specified similar user from the measured value storage unit 131 and the intake storage unit 132 and creates the prediction model (nutrition model) by multivariate analysis with the intake of nutrients as an explanatory variable and the measured value as an objective variable to specify the improvement factor that is a nutrient contributing to improvement of the measured value and the deterioration factor that is a nutrient contributing to deterioration of the measured value.

The evaluation unit 116 and the meal menu recommendation unit 117 can recommend a meal menu to the user similarly to the first embodiment.

### <Common to Patterns 1 and 2>

In Patterns 1 and 2, a meal menu is recommended using data or analysis result of one similar user, but in a case where improvement of a measurement result regarding the goal to be achieved by the user is not observed, another similar user may be selected.

For example, the similar user specifying unit 120 specifies a first similar user, the evaluation unit 116 evaluates a meal menu by using data or analysis result of the first similar user, the meal menu recommendation unit 117 recommends a meal menu that is expected to improve the measurement result of the user according to the evaluation, and after continuing such a process for a predetermined period (for example, one week or one month), the measured value acquisition unit 111 acquires whether or not the measured value relating to the goal such as the body weight or blood pressure is improved. The measured value acquisition unit 111 may conduct a questionnaire as to whether or not the measured value is improved for the user instead of acquiring the measured value. Here, in a case where the measured value is not improved (for example, in a case where the measured value has deteriorated, in a case where the measured value has not changed, or in a case where the measured value has improved by less than a predetermined value), the similar user specifying unit 120 specifies a second similar user different from the first similar user in the same manner as the first similar user (selects the second similar user according to the similarity of the attributes), the evaluation unit 116 evaluates a meal menu by using data or analysis result of the specified second similar user, and the meal menu recommendation unit 117 recommends the meal menu that is expected to improve the measurement result of the user according to the evaluation. This processing can be repeated until the improvement of the measured value of the user is observed.

### <Pattern 3: Using Analysis Result Obtained by Analyzing Data of Plurality of Other Users>

In Pattern 3, data on a plurality of similar users similar to the attributes of the user is analyzed.

The similar user specifying unit 120 can specify a plurality of (the number of similar users may be a predetermined number, or all or some of users having a predetermined degree of similarity or more may be selected at random) similar users having similar attributes from among other users who have registered the measured values relating to the goal to be achieved by the user in the measured value storage unit 131.

The analysis unit 115 can reads all the measured values and the intakes of nutrients corresponding to the specified similar users from the measured value storage unit 131 and the intake storage unit 132 and creates the prediction model (nutrition model) by multivariate analysis with the intakes of the nutrients as an explanatory variable and the measured value as an objective variable to specify the improvement factor that is a nutrient contributing to improvement of the measured value and the deterioration factor that is a nutrient contributing to deterioration of the measured value.

The evaluation unit 116 and the meal menu recommendation unit 117 can recommend a meal menu to the user similarly to the first embodiment.

### <Pattern 4: Using Analysis Results of Plurality of Other Users>

In Pattern 4, evaluation and recommendation of a meal menu are performed using analysis results of a plurality of similar users.

First, the similar user specifying unit 120 can specify a plurality of (the number of similar users may be a predetermined number, or all or some of users having a predetermined degree of similarity or more may be selected at random) users having similar attributes as similar users from among other users who have the same goal to be achieved as the goal to be achieved by the user.

Next, the analysis unit 115 can acquire a plurality of prediction models created for the plurality of similar users from the prediction model storage unit 138, and combine the acquired prediction models to create one prediction model (hereinafter, referred to as a combined model). For example, the analysis unit 115 can select an explanatory variable common to a plurality of prediction models (used for a predetermined number or more of prediction models) and use the selected explanatory variable as an explanatory variable of the combined model. For a coefficient of each explanatory variable, the analysis unit 115 can select a coefficient having the largest absolute value, select a median value of the coefficient, or calculate an average value of the coefficients, and can use the selected coefficient as a coefficient relating to the explanatory variable of the combined model. Furthermore, the analysis unit 115 may select an explanatory variable having the largest influence, for example, by using principal component analysis or the like.

The evaluation unit 116 and the meal menu recommendation unit 117 can recommend a meal menu to the user by using the combined model created as described above as the prediction model by the same method as in the first embodiment.

As described above, with a health support system according to the second embodiment, the user can easily achieve the goal without acquiring the intake of nutrients and the measured value linked to the goal to be achieved.

In the second embodiment, the nutrition model has been described, but a similar method can be used for the nutrition-activity model.

### REFERENCE SIGNS LIST

1 Server device
2 Communication network
3 User terminal
4 Vital sensor
5 Activity sensor
111 Measured value acquisition unit
112 Intake acquisition unit
113 Activity amount acquisition unit
114 Life stage information acquisition unit
115 Analysis unit
116 Evaluation unit
117 Meal menu recommendation unit
118 Activity recommendation unit
131 Measured value storage unit
132 Intake storage unit
133 Activity amount storage unit
134 Life stage information storage unit
135 Meal menu storage unit
136 Ingredient information storage unit

1. An information processing system comprising:
a measured value storage unit configured to store a measured value linked to a goal to be achieved by a user;
an intake storage unit configured to store intakes of a plurality of nutrients ingested by the user;
an analysis unit configured to analyze a relationship between the intake of the nutrient and the measured value to specify an improvement factor that is the nutrient contributing to improvement of the measured value and a deterioration factor that is the nutrient contributing to deterioration of the measured value;
a meal menu storage unit configured to store information specifying a content of the nutrient included in a meal menu; and
a meal menu recommendation unit configured to modify the meal menu by at least one of increasing the content of the improvement factor and decreasing the content of the deterioration factor, and recommend the modified meal menu.

2. The information processing system according to claim 1, further comprising an ingredient information storage unit configured to store the content of the nutrient for each ingredient,
wherein the meal menu storage unit stores the ingredient included in the meal menu, and
the meal menu recommendation unit recommends the modified meal menu by performing at least one of:
selecting, from the ingredient information storage unit, a second ingredient including a larger amount of the nutrient that is the improvement factor than at least one first ingredient included in the meal menu, and modifying the meal menu so as to change the first ingredient to the selected second ingredient;
selecting, from the ingredient information storage unit, a third ingredient including a smaller amount of the nutrient that is the deterioration factor than the first ingredient, and modifying the meal menu so as to change the first ingredient to the selected third ingredient;
selecting, from the ingredient information storage unit, a fourth ingredient including a larger amount of the nutrient that is the improvement factor than the first ingredient and including a smaller amount of the nutrient that is the deterioration factor than the first ingredient, and modifying the meal menu so as to change the first ingredient to the selected fourth ingredient;
selecting, from the ingredient information storage unit, a fifth ingredient including the nutrient that is the improvement factor, and modifying the meal menu so as to add the selected fifth ingredient; and
selecting, from the ingredient information storage unit, a sixth ingredient including the nutrient that is the deterioration factor, and modifying the meal menu so as to delete the selected sixth ingredient.

3. The information processing system according to claim 1 or 2, wherein
the analysis unit calculates a degree of improvement contributing to improvement for the improvement factor, and calculates a degree of deterioration contributing to deterioration for the deterioration factor, and
the meal menu recommendation unit
calculates a first value obtained by subtracting a value obtained by multiplying the degree of deterioration relating to the deterioration factor included in the meal menu by the content from a value obtained by multiplying the degree of improvement relating to the improvement factor included in the meal menu by the content, and
modifies the meal menu such that a second value obtained by subtracting a value obtained by multiplying the degree of deterioration relating to the deterioration factor included in the modified meal menu by the content from a value obtained by multiplying the degree of improvement relating to the improvement factor included in the modified meal menu by the content is larger than the first value.

4. The information processing system according to claim 1 or 2, wherein
the analysis unit calculates a degree of improvement contributing to improvement for the improvement factor, and calculates a degree of deterioration contributing to deterioration for the deterioration factor, and
the meal menu recommendation unit
calculates a first improvement value obtained by subtracting a value obtained by multiplying the intake relating to the deterioration factor stored in the intake storage unit by the degree of deterioration from a value obtained by multiplying the intake relating to the improvement factor stored in the intake storage unit by the degree of improvement, and
modifies the meal menu such that a second improvement value obtained by subtracting a value obtained by multiplying the degree of deterioration relating to the deterioration factor included in the modified meal menu by the content from a value obtained by multiplying the degree of improvement relating to the improvement factor included in the modified meal menu by the content is larger than the first improvement value.

5. The information processing system according to any one of claims 1 to 4, further comprising:
an activity amount storage unit configured to store an activity amount of an activity performed by the user; and
a target value input unit configured to receive an input of a target value of the measured value,
wherein the analysis unit creates a prediction model obtained by analyzing a relationship between the content of the nutrient, the activity amount, and the measured value, and
the meal menu recommendation unit determines the activity amount according to a difference between a predicted value obtained by applying a content of the nutrient included in the modified meal menu to the prediction model and the target value, and recommends the determined activity amount together with the meal menu.

6. The information processing system according to any one of claims 1 to 5, wherein the analysis unit periodically performs analysis to specify the improvement factor and the deterioration factor, and the meal menu recommendation unit recommends the modified meal menu.

7. An information processing system comprising:
a measured value storage unit configured to store a measured value for a first user linked to a goal to be achieved by the first user;
an intake storage unit configured to store intakes of a plurality of nutrients ingested by the first user;
an analysis unit configured to analyze a relationship between the intake of the nutrient and the measured value for the first user to specify an improvement factor that is the nutrient contributing to improvement of the measured value and a deterioration factor that is the nutrient contributing to deterioration of the measured value;
a meal menu storage unit configured to store information specifying a content of the nutrient included in a meal menu;
a similar user specifying unit configured to specify a similar user who is the first user similar to an attribute of a second user; and
a meal menu recommendation unit configured to modify the meal menu by at least one of increasing the content of the improvement factor specified for the similar user and decreasing the content of the deterioration factor specified for the similar user, and recommend the modified meal menu to the second user.

8. An information processing system comprising:
a measured value storage unit configured to store a measured value for a first user linked to a goal to be achieved by the first user;
an intake storage unit configured to store intakes of a plurality of nutrients ingested by the first user;
a meal menu storage unit configured to store information specifying a content of the nutrient included in a meal menu;
a similar user specifying unit configured to specify a similar user who is the first user similar to an attribute of a second user;
an analysis unit configured to read the measured value and the intake corresponding to the similar user from the measured value storage unit and the intake storage unit and analyze a relationship between the intake of the nutrient and the measured value to specify an improvement factor that is the nutrient contributing to improvement of the measured value and a deterioration factor that is the nutrient contributing to deterioration of the measured value; and
a meal menu recommendation unit configured to modify the meal menu by at least one of increasing the content of the improvement factor and decreasing the content of the deterioration factor, and recommend the modified meal menu to the second user.

9. An information processing method executed by a computer, the information processing method comprising:
storing a measured value linked to a goal to be achieved by a user;
storing intakes of a plurality of nutrients ingested by the user;
analyzing a relationship between the intake of the nutrient and the measured value to specify an improvement factor that is the nutrient contributing to improvement of the measured value and a deterioration factor that is the nutrient contributing to deterioration of the measured value;
storing information specifying a content of the nutrient included in a meal menu; and
modifying the meal menu by at least one of increasing the content of the improvement factor and decreasing the content of the deterioration factor, and recommending the modified meal menu.

10. A program for causing a computer to execute:
storing a measured value linked to a goal to be achieved by a user;
storing intakes of a plurality of nutrients ingested by the user;
analyzing a relationship between the intake of the nutrient and the measured value to specify an improvement factor that is the nutrient contributing to improvement of the measured value and a deterioration factor that is the nutrient contributing to deterioration of the measured value;
storing information specifying a content of the nutrient included in a meal menu; and
modifying the meal menu by at least one of increasing the content of the improvement factor and decreasing the content of the deterioration factor, and recommending the modified meal menu.
